SEQUENCE LISTING (14 sequences)

SEQ ID NO: 1 - protein, 492 aa, Mus musculus
  Amino acid sequence of murine ATG14L protein (1)..(492)
MASPSGKGSWTPEAPGFGPRALARDLVDSVDDAEGLYVAVERCPLCNTTRRRLTCAKCVQSGDFVYFDGRDRERFIDKKERLSQLKNKQEEFQKEVLKAMEGKRLTDQLRWKIMSCKMRIEQLKQTICKGNEEMKKNSEGLLKNKEKNQKLYSRAQRHQEKKEKIQRHNRKLGDLVEKKTIDLKSHYERLARLRRSHILELTSIIFPIDEVKTSGRDPADVSSETDSAMTSSMVSKLAEARRTTYLSGRWVCDDHNGDTSISITGPWISLPNNGDYSAYYNWVEEKKTTQGPDMEHNNPAYTISAALGYATQLVNIVSHILDINLPKKLCNSEFCGENLSKQKLTRAVRKLNANILYLCSSQHVNLDQLQPLHTLRNLMHLVSPRSEHLGRSGPFEVRADLEESMEFVDPGVAGESDASGDERVSDEETDLGTDWENLPSPRFCDIPSQPVEVSQSQSTQVSPPIASSSAGGMISSAAASVTSWFKAYTGHR

SEQ ID NO: 2 - protein, 941 aa, Mus musculus
  Amino acid sequence of murine Rubicon protein (1)..(941)
MRPEGAGMDLGGGDGERLLEKSRREHWQLLGNLKTTVEGLVSANCPNVWSKYGGLERLCRDMQNILYHGLIHDQVCCRQADYWQFVKDIRWLSPHSALHVEKFISLHESDQSDTDSVSERAVAELWLQHSLQCHCLSAQLRPLLGDRQYIRKFYTETAFLLSDAHVTAMLQCLEAVEQNNPRLLAQIDASMFARKQESPLLVTKSQSLTALPGSTYTPPASYAQHSYFGSSSSLQSMPQSSHSSERRSTSFSLSGPSWQPQEDRECLSPAETQTTPAPLPSDSTLAQDSPLTAQEMSDSTLTSPLEASWVSSQNDSPSDVSEGPEYLAIGNPAPHGRTASCESHSSNGESSSSHLFSSSSSQKLESAASSLGDQEEGRQSQAGSVLRRSSFSEGQTAPVASGTKKSHIRSHSDTNIASRGAAEGGQYLCSGEGMFRRPSEGQSLISYLSEQDFGSCADLEKENAHFSISESLIAAIELMKCNMMSQCLEEEEVEEEDSDREIQELKQKIRLRRQQIRTKNLLPAYRETENGSFRVTSSSSQFSSRDSTQLSESGSAEDADDLEIQDADIRRSAVSNGKSSFSQNLSHCFLHSTSAEAVAMGLLKQFEGMQLPAASELEWLVPEHDAPQKLLPIPDSLPISPDDGQHADIYKLRIRVRGNLEWAPPRPQIIFNVHPAPTRKIAVAKQNYRCAGCGIRTDPDYIKRLRYCEYLGKYFCQCCHENAQMVVPSRILRKWDFSKYYVSNFSKDLLLKIWNDPLFNVQDINSALYRKVKLLNQVRLLRVQLYHMKNMFKTCRLAKELLDSFDVVPGHLTEDLHLYSLSDLTATKKGELGPRLAELTRAGAAHVERCMLCQAKGFICEFCQNEEDVIFPFELHKCRTCEECKACYHKTCFKSGRCPRCERLQARRELLAKQSLESYLSDYEEEPTEALALEATVLETT

SEQ ID NO: 3 - protein, 492 aa, Homo sapiens
  Amino acid sequence of human ATG14L protein (1)..(492)
MASPSGKGARALEAPGCGPRPLARDLVDSVDDAEGLYVAVERCPLCNTTRRRLTCAKCVQSGDFVYFDGRDRERFIDKKERLSRLKSKQEEFQKEVLKAMEGKWITDQLRWKIMSCKMRIEQLKQTICKGNEEMEKNSEGLLKTKEKNQKLYSRAQRHQEKKEKIQRHNRKLGDLVEKKTIDLRSHYERLANLRRSHILELTSVIFPIEEVKTGVRDPADVSSESDSAMTSSTVSKLAEARRTTYLSGRWVCDDHNGDTSISITGPWISLPNNGDYSAYYSWVEEKKTTQGPDMEQSNPAYTISAALCYATQLVNILSHILDVNLPKKLCNSEFCGENLSKQKFTRAVKKLNANILYLCFSQHVNLDQLQPLHTLRNLMYLVSPSSEHLGRSGPFEVRADLEESMEFVDPGVAGESDESGDERVSDEETDLGTDWENLPSPRFCDIPSQSVEVSQSQSTQASPPIASSSAGGMISSAAASVTSWFKAYTGHR

SEQ ID NO: 4 - protein, 972 aa, Homo sapiens
  Amino acid sequence of human Rubicon protein (1)..(972)
MRPEGAGMELGGGEERLPEESRREHWQLLGNLKTTVEGLVSTNSPNVWSKYGGLERLCRDMQSILYHGLIRDQACRRQTDYWQFVKDIRWLSPHSALHVEKFISVHENDQSSADGASERAVAELWLQHSLQYHCLSAQLRPLLGDRQYIRKFYTDAAFLLSDAHVTAMLQCLEAVEQNNPRLLAQIDASMFARKHESPLLVTKSQSLTALPSSTYTPPNSYAQHSYFGSFSSLHQSVPNNGSERRSTSFPLSGPPRKPQESRGHVSPAEDQTIQAPPVSVSALARDSPLTPNEMSSSTLTSPIEASWVSSQNDSPGDASEGPEYLAIGNLDPRGRTASCQSHSSNAESSSSNLFSSSSSQKPDSAASSLGDQEGGGESQLSSVLRRSSFSEGQTLTVTSGAKKSHIRSHSDTSIASRGAPESCNDKAKLRGPLPYSGQSSEVSTPSSLYMEYEGGRYLCSGEGMFRRPSEGQSLISYLSEQDFGSCADLEKENAHFSISESLIAAIELMKCNMMSQCLEEEEVEEEDSDREIQELKQKIRLRRQQIRTKNLLPMYQEAEHGSFRVTSSSSQFSSRDSAQLSDSGSADEVDEFEIQDADIRRNTASSSKSFVSSQSFSHCFLHSTSAEAVAMGLLKQFEGMQLPAASELEWLVPEHDAPQKLLPIPDSLPISPDDGQHADIYKLRIRVRGNLEWAPPRPQIIFNVHPAPTRKIAVAKQNYRCAGCGIRTDPDYIKRLRYCEYLGKYFCQCCHENAQMAIPSRVLRKWDFSKYYVSNFSKDLLIKIWNDPLFNVQDINSALYRKVKLLNQVRLLRVQLCHMKNMFKTCRLAKELLDSFDTVPGHLTEDLHLYSLNDLTATRKGELGPRLAELTRAGATHVERCMLCQAKGFICEFCQNEDDIIFPFELHKCRTCEECKACYHKACFKSGSCPRCERLQARREALARQSLESYLSDYEEEPAEALALEAAVLEAT

SEQ ID NO: 5 - DNA, 1479 bp, Mus musculus
  Nucleotide sequence of murine ATG14L gene (1)..(1479)
atggcgtctcccagtgggaagggatcttggacgcccgaggctcctggttttgggccgcgggcgctagcacgggacctggtggactcggtggacgacgccgagggcctttacgtggctgttgagcggtgtcctctgtgcaacaccactcgccggcggttgacttgcgccaagtgcgtccagagcggtgatttcgtctatttcgacggccgcgaccgggagaggtttattgacaagaaggaaagactaagccaacttaagaacaagcaagaagaatttcagaaagaagtactaaaagctatggaaggaaagcggcttacagatcagttgagatggaaaataatgtcatgcaagatgaggattgaacagctgaagcaaacaatatgtaaaggaaatgaggaaatgaagaaaaattctgaaggtctcctcaagaacaaggaaaagaaccagaagctttacagccgagcacagcggcaccaagagaaaaaggagaagattcagcggcacaaccgcaagcttggggacctggtggagaagaagaccattgacttgaagagtcactatgagcggttggcgcggcttcgaaggtcacacatcctagagctcacctccatcatattcccaatcgacgaagtgaagacttctgggagagaccctgcagacgtgtcttcagagactgacagtgccatgacctcaagcatggtgagcaagcttgctgaggcccggaggacaacttacctctctggaagatgggtctgtgatgaccacaatggtgacaccagcattagcatcacaggcccgtggattagcctaccaaacaacggggactactctgcttactacaattgggtagaagagaagaaaacaacccaaggacctgacatggagcataacaaccccgcctacactatcagcgccgcgctgggctacgccacgcagctcgtcaacattgtgtctcacatacttgacatcaatcttcccaaaaagctgtgcaacagcgagttctgtggcgaaaacctcagcaagcagaaactgacacgcgcagtgaggaaactgaacgcaaacatcctttacctttgttcttctcagcatgtaaatctggatcagttgcaaccactgcacacactcaggaacctgatgcacttggtcagcccgcgctctgagcacctaggcaggtcaggaccctttgaagttcgagcagacctcgaggagtccatggaatttgtggaccctggagttgctggagaatcagacgcgagtggagatgagcgtgtaagcgatgaggagactgacctgggcacagactgggagaacctgccaagcccccgattctgtgacatcccttcccagccggtggaagtgtcccagagccagagcacccaggtgtccccacccattgccagcagcagcgctggtgggatgatctcctccgctgcggcctcggtgacctcttggttcaaagcttacactggacaccgctaa

SEQ ID NO: 6 - DNA, 1479 bp, Homo sapiens
  Nucleotide sequence of human ATG14L gene (1)..(1479)
atggcgtctcccagtgggaagggagcccgggcgctggaggctcctggctgcgggccccggccgctcgcccgggacctggtggactccgtggacgatgcggaggggctgtacgtggctgtggagcgctgcccgctgtgcaacactacccgccggcggctgacctgcgccaaatgcgttcagagcggcgatttcgtctacttcgacggccgcgaccgggagaggtttatcgacaagaaggaaaggttaagccgacttaagagcaagcaagaagaatttcagaaagaagtgttaaaagctatggaaggaaaatggataacagatcagttgagatggaaaataatgtcctgcaagatgaggattgaacagttaaaacaaacaatatgtaaaggaaatgaagaaatggagaaaaattctgaaggccttctcaaaaccaaggaaaagaatcagaagctttacagtcgagcacaacggcaccaagagaaaaaggagaagattcagaggcataatcgcaaacttggtgacctggtagaaaaaaagaccattgacttaagaagtcattatgagcgtctggcaaatcttcgacgatcccatatattagagctcacctctgtcatttttccaatcgaggaagtaaagacgggtgtgagagaccccgcagatgtgtcttcagagagtgacagtgccatgacctccagcactgtgagcaagcttgctgaagcccggaggacaacttacctctcaggacgatgggtctgtgacgatcacaacggagacaccagcattagcattacagggccttggattagcctccctaacaatggggactactctgcctactacagctgggtggaggagaagaaaacaacccaggggcctgacatggagcagagtaaccctgcctacaccatcagtgctgcgctgtgctatgcaactcagctggtcaacattctgtctcatatacttgatgtaaatcttcccaaaaagctctgcaacagtgaattttgtggcgaaaatctaagcaagcagaaatttactcgagcagtgaagaaactgaatgcaaatattctttacctttgtttttctcagcatgtaaatttagatcaattacaaccactgcataccctcaggaatctaatgtacctggtcagtccaagctctgaacacctaggcaggtcagggccctttgaagtacgagcagaccttgaggagtccatggaatttgtggatcccggagttgctggagaatcagatgagagcggagatgagcgcgtcagcgatgaagaaaccgacctgggcacagactgggagaacttgcctagtccccggttttgtgatatcccttcccagtctgtggaagtctcccagagtcagagcacccaggcgtccccacccatcgcgagcagcagtgcaggtgggatgatctcctctgcagcagcctcggtgacctcctggtttaaagcttacactggacaccgttaa

SEQ ID NO: 7 - DNA, 2826 bp, Mus musculus
  Nucleotide sequence of murine Rubicon gene (1)..(2826)
atgcgtccggagggcgcgggaatggatcttggcggcggcgatggggagcgtctgctagagaagagcaggagggagcactggcagctgctgggtaatttgaagacgactgtggaaggtttggtgtcagccaactgccccaatgtctggtccaagtatggtggcctggagcggctgtgcagggacatgcagaacatcctgtatcatgggctcatccatgaccaggtgtgttgccgccaggctgattactggcagtttgtgaaagacattcggtggctcagcccgcactcagcccttcacgtggagaagttcatcagtttgcatgagagcgaccagagcgacactgacagcgtgagtgagcgtgctgttgcagagctgtggctgcagcatagcctgcagtgccactgcctctcagcccagctccggcctctgctcggggacagacagtacatcagaaaattctacacagaaactgccttcctgctgagtgacgcccacgtcacagccatgctccagtgcctggaagcagtggaacagaacaacccccgtcttctggctcagatcgatgcatccatgtttgccagaaagcaggagagcccgctgctggtcacaaagagccagagtctgaccgctctgcctggttccacatacacccctccagctagctatgctcagcattcctactttgggtcctcctccagccttcagtctatgccacagtccagccacagctcagagagaagatctacttccttttcactctctggcccttcctggcaacctcaagaagacagagaatgcctctcaccagcagagactcaaaccaccccagctccgctgccttcagactctactctagcccaggattccccactgacagcacaggagatgagcgacagcactctgactagccccctagaggcatcctgggtcagcagccagaatgactccccaagtgatgtcagtgaggggcccgagtacctggccattggcaacccagcccctcatggccggactgccagttgtgagagtcacagcagcaacggcgagagcagcagctctcacttgttctcctccagcagctctcaaaagctggagtctgctgcctcttctctaggggaccaagaggaaggcaggcagagtcaggcaggcagcgtccttcgcaggtccagcttctcagaagggcagacagcccctgtcgccagcgggacaaagaagagccatattcgctcccactcggacaccaacattgcctccagaggagccgcagagggtggtcagtacctctgctcaggagaaggcatgtttagaagaccatcagaaggacagtccctcatcagttacctctctgagcaagactttggcagctgtgcagacctggaaaaggaaaacgcccacttcagcatctccgagtccttgattgccgccattgagctgatgaagtgcaacatgatgagccagtgtctagaagaggaggaagtagaggaggaagatagtgaccgagagatccaggaactgaagcagaagatccgccttcggcgccagcagatccgcaccaaaaacctgctccctgcgtaccgggagactgagaatggaagcttccgggtcacctccagcagctcccagttcagttcacgggattcgacgcagctctctgagtccggctctgctgaggatgctgacgacttggaaatccaagacgctgacatccggaggagtgcagtctcaaacggcaaatcatccttctcccagaatctctcgcactgcttcctgcactccacatcggctgaggcagtggccatggggcttctgaagcagtttgaggggatgcagcttccagctgcctcggagctcgagtggctagtcccagagcatgatgctccacagaagctcctacccatccccgactccctgcccatctcaccagatgacgggcagcacgcggacatctacaagctgcgaatccgcgtccgtggcaacctggagtgggctccgccccggccgcagatcatttttaatgttcatccagcccccacgaggaagattgccgtggccaagcagaattaccgctgtgcgggatgtggcatccggacggaccccgactatatcaagcggctgcggtactgtgagtacctaggcaagtacttctgccagtgctgccacgagaacgcccagatggtcgtccccagccgcattctgcgcaaatgggacttcagcaagtactatgtcagcaacttctccaaggacctgctcctgaagatctggaatgatcctctcttcaatgtgcaggacatcaatagcgcactctacaggaaggtcaagctgcttaaccaagtccggctgctgcgggttcagctgtaccacatgaagaacatgtttaagacatgccgactggccaaagagctcctggattcctttgacgtggttccaggccacctgacagaggacctccatctgtactcgctgagtgacctgactgcaaccaagaagggagaactggggccccggctggcggagctcactagagcgggagccgcccacgtggagagatgcatgctgtgtcaggccaagggcttcatctgtgaattctgtcagaatgaggaggatgtcatctttccttttgagctgcataagtgccggacttgtgaagagtgtaaagcgtgttaccataaaacttgcttcaagtctggacgctgcccccggtgtgagcggctgcaggcccgacgggagctgctggccaagcagagcctggagtcctacctgtctgactatgaggaggagcccacagaagccttggctctagaggccaccgtcctagagaccacctga

SEQ ID NO: 8 - DNA, 2919 bp, Homo sapiens
  Nucleotide sequence of human Rubicon gene (1)..(2919)
atgcggccggagggcgcgggaatggagctcggaggcggcgaggagcgcctgcctgaggagagcaggagggagcactggcagttgctgggtaatttgaagacgacggtggagggtttggtatcaaccaacagccccaacgtctggtctaagtatggtggcttggagcggctttgcagggacatgcagagcatcctctatcacgggcttatccgtgaccaggcgtgccgccgccagacggattactggcagttcgtgaaagacatccggtggctcagtccccactcagcccttcacgtggagaagttcatcagcgtgcacgagaacgaccagagcagtgctgatggtgccagtgaacgtgctgttgccgagctgtggctgcagcacagcctgcagtaccactgcctctcagcccagctccggcccctgctcggggatagacagtatatcagaaaattctacacagatgctgccttcctgctaagtgacgctcatgtcacggccatgctgcagtgcctggaagcagtggaacagaacaacccccgcctcctggctcagatcgatgcgtccatgtttgccagaaagcacgagagcccgctcctggtgacaaagagccagagcctgacagccctgcccagttccacatacacccctccaaacagctatgctcagcattcctactttgggtccttctctagcctccaccaatccgtgcccaacaatggctcagagagaagatctacttcctttccactctctggccctccccggaaacctcaagaaagcagagggcacgtctcaccagcagaggatcaaaccatccaagcccccccagtttcagtctctgcactagccagggattcccctttgaccccaaatgaaatgagctccagtactctgaccagccccatagaggcatcctgggtcagcagccagaatgattccccaggtgatgccagtgaggggcctgagtacctggccattggcaacttggacccccgaggccggactgccagctgtcagagtcacagcagcaatgccgagagcagcagttccaatttgttctcctccagcagctcccagaagccagattctgctgcctcttccttaggggaccaggaaggaggtggggagagccagctgtccagtgtcctccgcaggtccagcttctcagaggggcagacactcactgtcaccagtggggcaaagaaaagccacattcgctcccattcggataccagcattgcctccaggggagctccagaatcctgcaatgataaggcgaagttgagaggccctttgccctactctggtcaaagcagtgaagtcagcacacccagctctctgtacatggaatatgaaggtggtcggtacctgtgctcaggggaaggcatgttccgaagaccatcagaaggacagtccctcatcagctacctctctgagcaagacttcggcagctgtgccgacctggaaaaggagaatgcccacttcagcatctcagagtccttaattgctgccatcgagctaatgaagtgcaacatgatgagccagtgcctagaggaggaggaagtggaagaggaagacagtgatagagagatccaggagctgaagcagaagatccgccttcggcgccagcaaatccgcaccaagaacctgctccccatgtaccaggaggctgagcacggaagctttcgggtcacctccagcagctcccagttcagctcacgtgattcggcacagctctctgactctggctctgctgatgaggttgatgaatttgaaatccaagatgctgacatcagaaggaacacagcctcaagcagcaaatccttcgtttcctcccagtccttctcccactgcttcctgcactccacgtctgctgaggcggtggccatggggctcctgaagcagtttgaggggatgcagcttccagccgcctcggagctggagtggcttgtcccggagcatgatgcccctcagaagctcctgcccattcctgactcactgcccatctcaccggatgacgggcagcacgctgacatctacaagctgcggattcgtgttcgtggcaacttggagtgggccccgccccggcctcagataatttttaatgttcatccagccccaacgaggaaaattgccgtggccaagcagaattaccgctgtgcaggatgtggcatccggactgaccctgattacatcaagcgactgcggtactgtgagtacctgggcaagtacttctgccagtgctgccacgagaatgcccagatggccatccccagccgggttctgcgcaagtgggacttcagcaagtactacgtcagcaacttctccaaggacctgctcattaagatctggaatgatcctctcttcaacgtgcaggacataaacagtgccctctataggaaggtcaagctgctcaatcaagtccggctgctgcgggtccagctgtgtcacatgaagaacatgttcaagacttgccgactggccaaggagcttctggattcctttgacacagtcccaggccacctgacagaggacctccacctgtactcactgaatgacctgactgcgaccaggaagggggagctggggccccggcttgctgagctcaccagggcaggggctacccatgtggagagatgcatgctctgccaagccaaaggcttcatctgtgagttctgtcagaatgaggatgacatcatctttccctttgagctccataagtgccggacctgtgaagagtgtaaagcgtgttaccataaagcctgcttcaagtctggaagctgtccgcgctgcgagcggctgcaggcccggcgggaggcactggccaggcagagcctggagtcttacctgtcagactacgaggaggagcccgcggaagcgctggccctggaagccgccgtcctggaggccacctga

SEQ ID NO: 9 - protein, 450 aa, Homo sapiens
  Amino acid sequence of human Beclin protein (1)..(450)
MEGSKTSNNSTMQVSFVCQRCSQPLKLDTSFKILDRVTIQELTAPLLTTAQAKPGETQEEETNSGEEPFIETPRQDGVSRRFIPPARMMSTESANSFTLIGEASDGGTMENLSRRLKVTGDLFDIMSGQTDVDHPLCEECTDTLLDQLDTQLNVTENECQNYKRCLEILEQMNEDDSEQLQMELKELALEEERLIQELEDVEKNRKIVAENLEKVQAEAERLDQEEAQYQREYSEFKRQQLELDDELKSVENQMRYAQTQLDKLKKTNVFNATFHIWHSGQFGTINNFRLGRLPSVPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK

SEQ ID NO: 10 - protein, 448 aa, Mus musculus
  Amino acid sequence of murine Beclin protein (1)..(448)
MEGSKASSSTMQVSFVCQRCSQPLKLDTSFKILDRVTIQELTAPLLTTAQAKPGETQEEEANSGEEPFIETRQDGVSRRFIPPARMMSTESANSFTLIGEASDGGTMENLSRRLKVTGDLFDIMSGQTDVDHPLCEECTDTLLDQLDTQLNVTENECQNYKRCLEILEQMNEDDSEQLQRELKELALEEERLIQELEDVEKNRKVVAENLEKVQAEAERLDQEEAQYQREYSEFKRQQLELDDELKSVENQVRYAQIQLDKLKKTNVFNATFHIWHSGQFGTINNFRLGRLPSVPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK

SEQ ID NO: 11 - RNA, 25 nt, Artificial Sequence
  synthesized sequence
uuugcguucaguuuccucacugcgc

SEQ ID NO: 12 - RNA, 19 nt, Artificial Sequence
  synthesized sequence
gccuucagucuaugccaca

SEQ ID NO: 13 - DNA, 23 bp, Artificial Sequence
  synthesized sequence
cctacggcgtgcagtgcttcagc

SEQ ID NO: 14 - DNA, 25 bp, Artificial Sequence
  synthesized sequence
cggcgagctgcacgctgccgtcctc

(19) United States
(12) Patent Application Publication
(10) Pub. No.: US 2012/0041175 A1
(43) Pub. Date: Feb. 16, 2012

(54) NOVEL AUTOGRAPHY REGULATORS ATG14L AND RUBICON

(75) Inventor: Zhenyu Yue, New York, NY (US)

(73) Assignees: The Rockefeller Univeristy, New York, NY (US); Mount Sinai School of Medicine of New York University, New York, NY (US)

(21) Appl. No.: 13/046,428

(22) Filed: Mar. 11, 2011

Related U.S. Application Data
(63) Continuation of application No. PCT/US2009/056728, filed on Sep. 11, 2009.
(60) Provisional application No. 61/096,724, filed on Sep. 12, 2008.

(51) Int. Cl. C07K 14/47; C07H 21/00

(52) U.S. Cl. 530/350; 536/24.5

(57) ABSTRACT

The present invention provides for up- and down-regulation of cellular autophagy, e.g., for treating cancer or neurological disease. The invention results, in part, from discovery of two novel proteins, ATG14L (previously called “BISC”) and Rubicon (previously called “BIRC”), which bind to a Class III phophatidylinositol 3′-kinase (PI3K)/Vps34-Beclin 1 autophagic complex. ATG14L and Rubicon each regulate autophagic activity in an opposing manner. ATG14L and Rubicon can be used, for example, to increase/decrease autophagic activity, to increase/decrease PI3K/Vps34 kinase activity, and in so doing, treat diseases and disorders, such as cancer, neurodegenerative disease, stroke, metabolic disease, and age-related disease. ATG14L can increase autophagic activity and PI3K/Vps34 kinase activity; and Rubicon can decrease autophagic activity and PI3K/Vps34 kinase activity.

### CROSS-REFERENCE TO RELATED APPLICATIONS

This application is a continuation of International Application Serial No PCT/US09/056,728, filed Sep. 11, 2009, which claims priority from U.S. Patent Provisional Application Ser. No. 61/096,724, filed Sep. 12, 2008, each of which is hereby incorporated by reference in its entirety herein.

### GRANT INFORMATION

This invention was made with government support under NIH grant numbers 5R02NS060123-02, RNS055683A (Z.Y.), RR00862 and RR022220 awarded by National Institutes of Health. The United States Government has certain rights in the invention.

### SEQUENCE LISTING

The specification further incorporates by reference the Sequence Listing submitted herewith via EFS on Mar. 11, 2011. Pursuant to 37 C.F.R. §1.52(e)(5), the Sequence Listing text file, identified as 0701650655seqlisting.txt, is 46,715 bytes and was created on Mar. 11, 2011. The Sequence Listing, electronically filed herewith, does not extend beyond the scope of the specification and thus does not contain new matter.

### INTRODUCTION

The present invention relates to the discovery that proteins ATG14L and Rubicon may each be used to regulate autophagic activity, to regulate class III PI3K/Vps34 kinase activity, and to treat diseases and disorders, such as cancers, neurodegenerative diseases, inflammatory diseases, age-related diseases, and heart diseases.

### BACKGROUND OF THE INVENTION

Autophagy refers to a mechanism for breaking down cellular components including organelles or long-lived proteins in a cell. Autophagy is a process responsible for the bulk degradation of intracellular material that is evolutionarily conserved between all eukaryotes. In autophagy, cytoplasmic components are engulfed by double-membrane-bound structures (autophagosomes) and delivered to lysosomes/vacuoles for degradation. The autophagy process is mediated by a variety of proteins, including, among others, “Vps34”, “LC3”, and “p62.”

The morphology of autophagy was first characterized in studies of mammalian cells. With a few exceptions, however, the molecular components of autophagy were initially elucidated in yeast because of the convenience of gene analysis in that organism. Recent studies in various eukaryotic systems have revealed a conservation of the autophagic mechanism (Mizushima N et al., 2002). In the past, the many terms used in the autophagy field have been highly confusing: Aut (autophagocytosis), Apg (autophagy), Vps (vacuolar protein-sorting) all have been used. Recently, the autophagy-related genes and the products of these genes were named ATG and Atg, respectively (Klionsky D J et al., 2003).

In yeast, more than thirty genes are shown to be essential for autophagy (Tsukada M. and Ohsumi Y., FEBS Lett (1993); 333, 169-74). These ATG (AuTophagy-related) genes contribute either to the formation of the machinery for autophagy, or to its regulation in response to a variety of signals (Suzuki K. and Ohsumi Y., FEBS Letters (2007); 581, 2156; Klionsky D. J. et al., Developmental Cell (2003); 5, 539-45). In contrast, only a few of mammalian autophagy genes were identified, and the knowledge of the autophagic process in mammals is limited. In order to elucidate details of mammalian autophagy, it is important to determine whether many yeast ATG genes have homologues in mammals, and whether there are additional components of the autophagy machinery that are specific for mammals. Beclin 1 is the homologue of yeast ATG6/Vps30, and it is among the earliest characterized mammalian autophagy genes (Liang X. H. et al., Nature (1999); 402, 672). Beclin 1 is required for autophagosome formation and is monoallelically deleted in a high percentage of sporadic human breast, ovarian, and prostate carcinomas. In the established breast carcinoma cell line MCF7, the expression of Beclin 1 protein is decreased below a detectable level. Stable transfection of beclin 1 in MCF7 cells promotes autophagic activity and reduces tumorigenic capacity, which suggests that autophagic activity is associated with inhibition of cellular proliferation (Liang X H et al., 1999). In addition, beclin 1^{+/-} heterozygous mice suffer from a high incidence of spontaneous tumors, and beclin 1^{-/-} homozygous embryonic stem cells exhibit a decreased number of autophagic vesicles (Qu X et al., 2003; Yue Z et al., 2003). Autophagy may thus instigate tumor suppression, by causing cell death or by limiting cell growth.

Initially identified as a Bcl-2 binding protein (Liang X. H. et al., J. Virol. (1998); 72, 8586-8596), Beclin 1 has been shown both in vitro and in vivo to participate in the regulation of autophagy and to play an important role in development (Yue Z. et al., Proceedings of the National Academy of Sciences of the United States of America (2003); 100, 15077-82), tumorigenesis (Liang X. H. et al., Nature (1999); 402, 672; Yue Z. et al., Proceedings of the National Academy of Sciences of the United States of America (2003); 100, 15077-82; Qu X. et al., J. Clin. Invest. (2003); 112, 1809-1820; Edinger A. L. and Thompson C. B., Cancer Cell (2003); 4, 422), and neurodegeneration (Yue Z. et al., Neuron (2002); 35, 921 (2002); Diskin T. et al., J Neurotrauma (2005); 22, 750-621; Shibata M. et al., J Biol Chem (2006); 281, 14474-85; Pacheco C. D. et al., Hum Mol Genet (2007); 16, 1495-503). Despite the emerging evidence implicating Beclin 1 in various physiological and pathological conditions, the precise roles for Beclin1 in these processes remain to be defined.

Similar to Atg6 in yeast, Beclin1 also forms a complex with phosphatidylinositol (PtdIns) 3-kinase (Vps34, class III PI-3K) (Kihara A. et al., EMBO Rep (2001); 2, 330-5; Kihara A. et al., J Cell Biol (2001); 152, 519-30; Zeng X. et al., J Cell Sci (2006); 119, 259-70). In yeast, there are at least two distinct Atg6/Vps34 protein complexes: one containing Atg14 and participating in autophagy, and the second containing Vps38 (while lacking Atg14) and functioning in non-autophagic pathways (Kihara A. et al., J Cell Biol (2001); 152, 519-30). In contrast, no concrete evidence had hitherto suggested the presence of multiple Beclin 1-Vps34/PtdIns3K protein complexes or multiple functions associated with Beclin 1-Vps34/PtdIns3K complex in mammals (Zeng X. et al., J Cell Sci (2006); 119, 259-70). Recently, several novel Beclin 1-associated proteins (e.g., UVRAG, Ambra1 and Bif-1) have been identified in mammalian cells, providing new information with respect to the physiological functions of Beclin 1 (Liang C. et al., Nat Cell Biol (2006); 8, 688-99; Takahashi Y. et al., Nat Cell Biol (2007); 9, 1142-51; Fimia G. M. et al., Nature (2007); 447, 1121-5). Given these new data, and the roles of Beclin1 in a wide variety of physiologic responses, it becomes increasingly important to identify the biochemical components of the Beclin 1-Vps34/PtdIns3K complex and to understand their contributions to Beclin 1-mediated autophagy regulation.

In yeast, autophagy almost completely shuts off under growing conditions, although every ATG gene is expressed. Molecular biological and biochemical analyses of these gene products uncovered the genetic and biophysical interactions among the Atg proteins. One of the most remarkable findings regarding the Atg proteins is the discovery of two ubiquitin-like conjugation systems, Atg12-Atg5 and Atg8-phosphatidylethanolamine (PE). In fact, half of the APG genes essential for autophagy are involved in these conjugation systems, and these two conjugation systems are well conserved among eukaryotes. Furthermore, Atg12-Atg5 and Atg8 conjugation systems are somehow related to each other (Kim J et al., 2001; Mizuhima N et al., 2001; Suzuki K et al., 2001): if the former is defective, the latter cannot target to the pre-autophagosomal structure (PAS); the levels of Atg8-PE also play an important role in the conjugation of Atg12-Atg5.

Atg12-Atg5

Atg12, a small hydrophilic protein of 186 amino acids with no apparent homology to ubiquitin, can covalently link to a unique target protein, Atg5 (Mizushima N et al., 1998). The mode of conjugation of Atg12 and Atg5 is quite similar to that of ubiquitination. Atg12 is first activated in an ATP-dependent manner by Atg7 (it functions as an ubiquitin-activating enzyme, E1), leading to the formation of a thioester bond between the C-terminal glycine in Atg12 and a cysteine residue in Atg7 (Tanida I et al., 2001). The C-terminal glycine in Atg12 is then transferred to the cysteine in Atg10 (it functions as an ubiquitin-conjugating enzyme, E2), forming a new thioester bond, and Atg7 is released (Shintani T et al., 1999). Finally, the C-terminal glycine in Atg12 forms an isopeptide bond with the e-amino group of lysine 149 in Atg5, and Atg10 is in its free state again. The formation of the Atg12-Atg5 conjugate is believed to be required for autophagosome formation. It seems that this ubiquitin-like system is a constitutive process, because the formation of the Atg12-Atg5 conjugate is not dependent upon starvation or other autophagy-inducing conditions (Mizushima N et al., 1999). Atg12 and Atg5 form a conjugate immediately after their synthesis, and free forms of these are hardly detectable. The conjugation reaction between Atg5 and Atg12 is irreversible, and so far no protease has been found to deconjugate this conjugate. Atg16 also binds preferentially to the Atg12-Atg5 conjugate. Atg16 links with Atg12-Atg5 through self-oligomerization, and its C-terminal coiled-coil region may be responsible for this oligomerization. Therefore, Atg16 forms a 350 kDa multimeric complex with the Atg12-Atg5 conjugates. It should be pointed out that Atg16 only binds to Atg5, and not to Atg12 (Mizushima N et al., 2003; Kuma A et al., 2002). This new complex is necessary for the elongation of the isolation membranes (used for formation of the autophagosomal membrane). A small fraction of the Atg12-Atg5•Atg16 complex initially associates with a small crescent-shaped vesicle evenly. As the membrane elongates, Atg12-Atg5 shows asymmetric localization and most of these proteins associate with the convex surface of the isolation membrane. This complex will dissociate from the membrane upon completion of autophagosome formation; thus, it is not present in the mature autophagosome (George M D et al., 2000). The molecular basis of this transient association of Atp12-Atg5 conjugates with the autophago-some membrane is not yet known.

In mammalian cells, Atg5 and Atg12 are conjugated to each other in essentially the same way as they are in yeast, but the complex interacts with Atg16L, forming an ˜800 kDa structure instead of a 350 kDa complex in yeast (Mizushima N et al., 2003). Atg16L is a 63 to 74 kDa protein, which has a binding region and coiled-coil region similar to that of Atg16. However, Atg16L has a long C-terminal extension containing 7 WD repeats, but the role of the WD repeats in autophagy has not yet been elucidated.

Atg8 Conjugation System

The second ubiquitin-like protein essential for autophagy is Atg8 (Aut7/Apg8). Atg8 is a 117-amino acid protein and is present in the early isolation membranes, autophagosomes and autophagic bodies (Kirisako T et al., 1999). This feature makes Atg8 a good marker for studying membrane dynamics during autophagy. Microtubule-associated protein 1 light chain 3 (LC3), the mammalian orthologue of Atg8, targets to the autophagosomal membranes in an Atg5-dependent manner and remains there even after Atg12-Atg5 dissociates. Thus LC3 is a marker of the autophagosome in mammalian cells (Yoshimori T et al., 2004). In wild-type cells, LC3 is detected in 2 forms: LC3-I (18 kDa) and LC3-II (16 kDa) (Kabeya Y et al., 2000). Twenty-two amino acids in the C-terminus of the newly synthesized LC3 are cleaved immediately by the mammalian orthologue of the yeast cysteine proteinase Atg4, autophagin, to produce an active cytosolic form, LC3-I (Kabeya Y et al., 2004). Then with the catalysis of Atg7 and Atg3, LC3-I undergoes a series of ubiquitination-like reactions, and is modified to LC3-II. LC3-I is located in the cytoplasm, while LC3-II is a tightly membrane bound protein and is attached to PAS and autophagosomes. The relative amount of membrane-bound LC3-II reflects the abundance of autophagosomes, so the induction and inhibition of autophagy can be monitored through measuring total and free LC3-II levels by means of immunoassay (Kabeya Y et al., 2000). In addition, studies have shown that the Atg12 and LC3 systems have a functional relationship. In ATG5^{-/-} cells, LC3-II is not generated at all (Mizushima N et al., 2001). As a result, LC3 cannot target the autophagosomal membranes. The recent generation of transgenic mice expressing green fluorescent protein (GFP) fused to LC3 provides a useful tool to investigate autophagy in various mammalian organs in vivo (Mizushima N et al., 2004).

In addition to LC3, at least another two mammalian orthologs of yeast Atg8 have been identified (Sagiv Y et al., 2000; Wang H et al., 1999): g-aminobutyric acid type A receptor-associated protein (GABARAP) and Golgi-associated ATPase enhancer of 16 kDa (GATE-16). The two proteins also covert to membrane bound forms (form II), which are recovered in membrane fractions (Marino G et al., 2003). These results suggest that all mammalian Atg8 homologues receive common modifications to associate with autophagosomal membranes, but the functions of these orthologs and their modified form II need to be further studied.

Tracking the conversion of LC3-I to LC3-II is indicative of autophagic activity. Immunoblotting of LC3 usually reveals two bands: LC3-I (18 kDa) and LC3-II (16 kDa). The amount of LC3-II correlates well with the number of autophagosomes. This characteristic conversion of LC3 can be used to monitor autophagic activity. During autophagy, the cytoplasmic form (LC3 I) is processed and recruited to the autophagosomes, where LC3 II is generated by site specific proteolysis and lipidation near to the C-terminus. The hallmark of autophagic activation is thus the formation of cellular autophagosome punctae containing LC3 II, while autophagic activity is measured biochemically as the amount of LC3 II that accumulates in the absence or presence of lysosomal activity.

PI3K/Vps34 Complex

Vacuolar protein sorting 34 (Vps34) is a member (Class III) of the PI3K (phosphoinositide 3-kinase) family of lipid kinases, all of which phosphorylate the 3′ hydroxy position of the phosphatidylinositol ring. In the accepted nomenclature for PI3Ks (Fruman et al., 1998; Vanhaesebroeck et al., 2001), Vps34 is classified as the sole Class III enzyme, whose substrate specificity is limited to phosphatidylinositol. Thus its product in cells is PtdIns3P. This distinguishes it from the more numerous and better studied Class I and Class II enzymes, which can produce PtdIns3P, PtdIns (3,4)P2 or PtdIns(3,4,5)P3, depending on the isoform (Fruman et al., 1998; Vanhaesebroeck et al., 2001).

The first known functions of Vps34 were in the regulation of vesicular trafficking in the endosomal/lysosomal system, where it is involved in the recruitment of proteins containing PtdIns3Pbinding domains to intracellular membranes (Odorizzi et al., 2000; Lindmo et al., 2006). However, Vps34 has also been implicated in other signalling processes, including nutrient sensing in the mTOR [mammalian TOR (target of rapamycin)] pathway in mammalian cells, trimeric G-protein signalling to MAPK (mitogen-activated protein kinase) in yeast, and autophagy in both yeast and higher organisms (Byfield et al, 2005; Nobukuni et al., 2005; Slessareva et al., 2006; Kihara et al., 2001; and Petiot et al., 2000).

Vps34 function is regulated by the protein kinase activity of Vps15, with which it forms a stable, membrane-associated complex under normal conditions. This complex links the Vps34 kinase to cytoplasmic membranes. Vps34-Vps15 is present in two complexes, which are involved in a variety of membrane transport events (Kihara A et al., 2001): complex I, which is composed of Vps34-Vps15, Atg6 and Atg14, controls autophagy, whereas complex II, which is composed of Vps34-Vps15, Atg6 and Vps38, is essential for sorting of carboxypeptidase Y (CPY) into the vacuole. Atg6 is a possible coiled-coil protein and is associated with the membrane through Vps15 and Vps34. Atg14 is a specific factor in the autophagy-specific PI3K complex. Complex I functions primarily, but not exclusively, at the PAS, whereas complex II functions at the endosome. The lipid kinase activity associated with Vps34 is thought to create lipid patches of PI3-P, the reaction product of class III PI3K, at specific trans-Golgi locations, and these patches then function in protein sorting into vesicles that travel from the Golgi to the endosome.

In mammalian cells, there are three classes of PI3K. Class I PI3K is an inhibitor of autophagy. Class II PI3K activity is thought to have no relevance to autophagy control. Class III PI3K, a functional orthologue of yeast Vps34, is an activator of autophagy and plays a crucial role at an early step of autophagosome formation in mammalian cells (Arico S et al., 2001), and it is required for increasing the size of the sequestering membrane, presumably through fusion events. PI3-P interacts with proteins containing FYVE or PX motifs, thus recruiting such proteins from the cytosol for autophagosome biogenesis (Wurmser A E et al., 2002). The activation of a population of PI3K located in a determined membrane domain may be responsible for autophagosome biogenesis. In addition, the presence of PI3-P in a specific membrane location may generate significant asymmetries and drive membrane curvature of PAS (Tassa A et al., 2003). Finally, PI3-P may be converted to higher-order polyphos-phoinositides (PI), which are involved in diverse signaling functions. The mammalian cell orthologue of Vps15 is p150. It is associated with class III PI3K, and interacts with beclin-1, a functional orthologue of yeast Atg6 in mammalian cells (Liang X H et al., 1999).

### Autophagy and Cancer

Autophagy probably factors into both the promotion and prevention of cancer, and its role may be altered during tumor progression. Inhibition of autophagy may allow the continuous growth of precancerous cells, and autophagy can act as a suppressor of cancer (Gozuacik D et al., 2004; Orier-Denis E et al., 2003). Later, as a tumor grows, cancer cells may need autophagy to survive nutrient-limiting and low-oxygen conditions, especially in the internal region of the tumor that is poorly vascularized (Cuervo A M 2004)). In addition, autophagy may protect some cancer cells against ionizing radiation (Paglin S et al., 2001), possibly by removing damaged macromolecules or organelles, such as mitochondria, which could protect against apoptosis and allow continued survival of transformed cells (Alva A S et al., 2004).

The class I phosphatidylinositol (PtdIns) 3-kinase/protein kinase B (Akt/PKB) signaling pathway promotes cell growth in response to mitogenic signals, and mutations in several proteins in this pathway cause a high percentage of common human malignancies (Blume-Jensen P et al., 2001). Class I PtdIns 3-kinase generates PtdIns(3,4)P2 and PtdIns(3,4,5)P3, which bind to the pleckstrin homology domain of Akt and its activator 3-phosphoinositide-dependent protein kinase-1 (PDK-1) (Meijer A J et al., 2004). When the Akt signaling pathway is activated, autophagic degradation is reduced (Arico S et al., 2001)). Conversely, a dominant negative form of Akt induces a high rate of autophagy. Akt and PDK-1 activate other kinases, including mammalian target of rapamycin (mTOR), which negatively regulates autophagy. The tumor suppressor PTEN, which has a 3′-phosphoinositide phosphatase activity and antagonizes the PtdIns 3-kinase/Akt pathway, positively regulates autophagy. Mutations in PTEN result in constitutive activation of the Akt signaling pathway and inactivation of autophagy and lead to tumor formation (Arico S et al., 2001). Because Akt is centrally involved in regulating a range of substrates that participate in cell growth and survival (Hanada M et al., 2004), tumorigenesis resulting from activation of Akt may be due to a block in a pathway other than autophagy.

Some pharmaceuticals used to treat cancer are likely to act through autophagy. For example, tamoxifen is used to treat certain types of breast cancer and may function by activating autophagy, possibly through the up-regulation of beclin 1 in a process mediated by ceramide (Scarlatti F et al., 2004). Other autophagy-inducing compounds, including the mTor inhibitor rapamycin and various analogs, are currently being tested in clinical trials in patients with malignant tumors, although the antitumor effects of inhibiting mTor could reflect its role in cell cycle regulation or translation rather than autophagy.

Autophagy and Neurodegenerative Diseases

Early reports demonstrating that autophagosomes accumulate in the brains of patients with diverse neurodegenerative diseases, including Alzheimer's disease, transmissible spongiform encephalopathies, Parkinson's disease, and Huntington's disease (Rubinsztein et al., 2007; Williams et al., 2006), led to the initial hypothesis that autophagy contributed to the pathogenesis of these disorders. In mice with cerebellar degeneration due to mutations in the glutamate receptor, autophagy was also postulated to be a mechanism of nonapoptotic cell death (Yue et al., 2002). In contrast, more recent studies provide compelling evidence that at least in model organisms autophagy protects against diverse neurodegenerative diseases and that the accumulation of autophagosomes primarily represents the activation of autophagy as a beneficial physiological response or, in the case of Alzheimer's disease, the consequence of a defect in autophagosomal maturation (Martinez-Vicente and Cuervo, 2007; Rubinsztein et al., 2007; Williams et al., 2006).

Beyond its role in the clearance of misfolded proteins spontaneously generated during routine protein turnover, autophagy likely plays an important role in the clearance of aggregate-prone mutant proteins associated with several different neurodegenerative diseases. These include proteins with polyglutamine (polyQ) expansion tracts such as those seen in Huntington's disease and spinocerebellar ataxia, mutant α-synucleins that cause familial Parkinson's disease, and different forms of tau including mutations associated with frontotemporal dementia (Williams et al., 2006). Because substrates need to be unfolded to pass through the narrow pore of the proteasomal barrel, oligomeric and aggregated proteins are poor substrates for proteasomal degradation and better targets for autophagic degradation. The mechanism by which these proteins exert their cellular toxicity is still controversial, but it is generally believed that they are particularly toxic in oligomeric complexes and that higher-order protein aggregates may be formed as a last attempt to prevent toxicity in the absence of a properly functioning quality-control system (Martinez-Vicente and Cuervo, 2007). This view is consistent with the model that autophagy functions as a quality-control system that targets oligomeric proteins and with the evidence that autophagy activation reduces, whereas autophagy inhibition increases, the formation of protein aggregates and the neurotoxicity of aggregate-prone proteins. Pharmacological activation of autophagy reduces the levels of soluble and aggregated forms of mutant huntingtin protein, proteins mutated in spinocerebellar ataxia, mutant forms of α-synuclein, and mutant tau; it also reduces their cellular toxicity in vitro and their neurotoxicity in either mouse or Drosophila models (Rubinsztein et al., 2007). ATG gene knockdown or knockout increases aggregate formation and toxicity of polyQ expansion proteins in C. elegans (Jia et al., 2007). Autophagy induced by overexpression of histone deacetylase 6 also compensates for impairment in the ubiquitin-proteasome system in a fly model of spinobulbar muscle dystrophy (Pandey et al., 2007). In these models, autophagy-mediated neuroprotection may be due to a quantitative reduction in the amounts of the toxic protein species as well as antiapoptotic effects (Rubinsztein et al., 2007). The development of neurodegenerative disease in patients with proteinopathies implies that the autophagy may reach a saturation point in which its capacity to degrade the mutant aggregate-prone proteins is exceeded, or that concurrent defects may occur in the autophagy pathway. Acquired defects in autophagosome formation may result from the sequestration of autophagy proteins in aggregates formed by mutant proteins, the age-related decline that occurs in Beclin 1 and potentially other autophagy protein expression in human brain, and other as-of-yet unidentified factors (Shibata et al., 2006). In addition to defects that result in decreased autophagic activity, genetic or functional alterations may occur that impair delivery of autophagosomes to the lysosome. For example, mutations that affect the dynein motor machinery impair autophagosome-lysosome fusion, leading to decreased autophagic clearance of aggregate proteins and enhanced toxicity of the huntingtin mutant protein in Drosophila and mouse models (Ravikumar et al., 2005). It is thus possible that impaired autophagic clearance may contribute to the pathogenetic mechanism by which dynein mutations cause motor neuron diseases in humans. Other mutations involved in lysosomal function also reduce autophagic turnover (with an observed increase in autophagosomal accumulation) and are associated with neuronal ceroid lipofuscinosis (Batten disease) in mice, such as CLN3 partial deletion, cathepsin D knockout, and double cathepsin B/L knockout. The interrelationship between autophagy and Alzheimer's disease may also involve an impairment in autophagolysosomal maturation. Autophagosome-like structures accumulate in dystrophic neurons of Alzheimer's disease patients, presumably as a result of impairment in autophagolysosomal maturation and, intriguingly, may constitute a unique site of production/accumulation of the pathogenic amyloid β protein (Yu et al., 2005).

The role of autophagy in protection against neurodegenerative diseases is established in animal models but not yet in patients. Nonetheless, preclinical animal data provide a strong rationale for proceeding with clinical trials with autophagystimulatory agents; this is especially true as agents shown to be beneficial in reducing neurotoxicity of mutant aggregate-prone proteins are already in clinical use to treat other diseases. Rapamycin analogs, which are approved for the use of preventing organ transplant rejection and postangioplasty coronary artery restenosis and are in phase II oncology trials, protect against neurodegeneration seen in Drosophila and mouse polyQ disease models (Rubinsztein et al., 2007). However, because TOR also affects protein synthesis, cell proliferation, cell growth, cell death, and immune function, its inhibition has some adverse effects; perhaps intermittent rather than continuous stimulation of autophagy with rapamycin may reduce such side effects while maintaining therapeutic efficacy. Lithium chloride, a drug used for the treatment of bipolar disorder, induces autophagy by reducing IP3 levels and enhances the clearance of aggregate-prone proteins (Rubinsztein et al., 2007). Another group of new agents, small molecule enhancers of rapamycin (SMERs), enhance the clearance of mutant huntingtin and α-synuclein and protect against neurodegeneration in a Drosophila Huntington's disease model (Sarkar et al., 2007). Given that lithium and SMERs both act independently of TOR, it is possible that they may be used therapeutically in combination with rapamycin analogs. Of long-term interest, both for the treatment of neurodegenerative diseases and, potentially, the prevention of aging, would be drugs that can reverse age-dependent declines that occur in CNS autophagy protein expression and lysosomal clearance of autophagosomes.

Autophagy and Inflammatory Diseases

Autophagy has been found to occur in ATP-dependent generation of engulfment signals and heterophagic removal of apoptotic corpses (Qu et al., 2007). The rapid removal of apoptotic corpses is critical for the prevention of tissue inflammation (Grossmayer et al., 2005), and indeed, autophagy-deficient atg5^{-/-} embryos have increased inflammation in tissues that have impaired clearance of apoptotic cells (Qu et al., 2007). Moreover, the lack of efficient apoptotic cell clearance may overcome tolerance to self-antigens and lead to autoimmune diseases such as systemic lupus erythematosis (SLE) (Grossmayer et al., 2005).

Several recent genome-wide scans have uncovered strong genetic associations between two genes involved in autophagy, the autophagy-stimulatory immunity-related GTPase, IRGM1, and the autophagy execution gene, Atg16L, and susceptibility to Crohn's disease, a chronic inflammatory disease of the intestine. These studies suggest a potential role for autophagy deregulation in the pathogenesis of Crohn's disease. However, as of yet, it is not known whether the ATG16L variant (T300A) is defective in autophagy function and whether this genetic link is indicative of an underlying mechanistic role of autophagy impairment in Crohn's disease pathogenesis. The pathogenic mechanisms of Crohn's disease are incompletely understood but are postulated to involve a dysregulated immune response to commensal intestinal bacteria, altered mucosal barrier function, and/or defects in bacterial clearance (Baumgart and Carding, 2007). It is speculated that autophagy deficiency might contribute to one or more of these pathological mechanisms in Crohn's disease (Levine and Deretic, 2007). Studies in targeted mutant mice with conditional deletion of atg16 and other ATG genes should help elucidate potential interrelationships between alterations in autophagy and the pathogenesis of Crohn's disease.

Autophagy and Age-Related Diseases

Recent genetic analyses in various organisms have resulted in the identification of genes involved in controlling longevity. The best-characterized pathway is the insulin/insulin-like growth factor 1 (IGF-1) pathway, which is highly conserved in eukaryotes from yeast to human (Longo V D & Finch C E, 2003). This signaling cascade includes a tyrosine kinase receptor, PtdIns 3-kinase and Akt/PKB, all of which are also involved in tumorigenesis as described above. In the case of the nematode Caenorhabditis elegans, inactivation of this cascade extends life-span up to 300% and increases heat and oxidative stress resistance, which may contribute to life-span extension. Because Akt/PKB controls the activity of Tor, the autophagy inhibitor, the down-regulation of the Akt/PKB pathway may also induce autophagy, so that autophagy may partly account for life-span extension. Along these lines, elimination or depletion of the TOR kinase/LET-363 also results in an increase in life-span (Vellai T et al., 2003). A loss-of-function mutation in the insulin-like tyrosine kinase receptor daf-2 extends life-span in C. elegans twofold; however, the inactivation of bee-1, the nematode ortholog of the yeast and mammalian VPS30/ATG6/beclin 1 gene that is essential for autophagy, by RNAi cancels this effect, which suggests that autophagy is required for the life-span extension resulting from down-regulation of the insulin/IGF-1 pathway (Melendez A et al., 2003). This agrees with the finding that the life-span extension resulting from RNAi-mediated inhibition of TOR is not additive with daf-2 (Vellai T et al., 2003).

The rate of autophagy decreases with age (Bergamini E et al., 2004), which suggests a possible correlation between the two processes. Caloric restriction, which might induce autophagy, has positive effects on life-span extension (Longo V D & Finch C E, 2003; Bergamini E et al., 2004). The increase in longevity may be brought about by an increase in protection against oxidative damage, such as through the removal of damaged mitochondria (Terman A et al., 2004), as well as by mechanisms involved in the repair and replacement of damaged DNA, proteins and lipids.

A corollary question is whether autophagy function declines with age, and if so, whether such a decline contributes to aging and susceptibility to age-related diseases. Indeed, both classical autophagy (macroautophagy) and chaperone-mediated autophagy decline with aging in rodents and probably in humans (Martinez-Vicente and Cuervo, 2007). The mechanism for the decline in autophagy with aging is unknown but, at least in the rodent liver, is thought to involve alterations both in responses to hormonal regulation of autophagy (e.g., glucagon, insulin) and in the degradation of autophagosomes (Del Roso et al., 2003). It is possible that the accumulation of undigested material inside secondary lysosomes—a characteristic feature of aged, postmitotic cells—interferes with the ability of lysosomes to fuse with autophagosomes and degrade their cargo, thus creating a vicious cycle leading to a progressive defect in autophagosome degradation. However, it is equally plausible that this feature of aging results from, rather than causes, autophagy impairment. It will be important to further determine what age-related changes occur in the expression or function of proteins directly involved in autophagy regulation and execution or in lysosomal function.

A cardinal feature of aging postmitotic cells is the accumulation of damaged proteins and organelles—especially functionally disabled mitochondria and lipofuscin-loaded lysosomes. Mutations that decrease mitochondrial metabolism or reduce levels of reactive oxygen species extend life span in C. elegans and rodents, presumably by decreasing the generation of damaged proteins and organelles (Levine and Klionsky, 2004). In parallel, the activation or inhibition of autophagy likely prevent or promote aging, respectively, due to alterations in the removal of damaged proteins and organelles. As noted above, tissue-specific deletion of ATG genes in postmitotic cells, including neurons, hepatocytes, and cardiomyocytes, reveals a critical role for basal autophagy in protein and organelle turnover and prevention of cellular degeneration. Therefore, it is easy to imagine how either the cumulative effects of incomplete autophagic clearance over prolonged periods and/or age-related declines in autophagy function could contribute to aging. Caloric restriction can extend life span in diverse species and reverse the age-related decline in autophagy in rodent liver (Kenyon, 2005). Yet, despite the practice of fasting to promote longevity throughout human history, alternative approaches should be sought that mimic the beneficial effects of caloric restriction on autophagy while avoiding the potential adverse effects of caloric restriction on other aspects of human health. One such candidate is an antilipolytic drug that has been used successfully to increase autophagic activity in rodent liver and to extend life span (Bergamini, 2005). The life-span effects of other clinically used autophagy inducers such as rapamycin and lithium chloride have not been investigated. Ideally, the molecular basis for the age-related decline in autophagy function should be identified and pharmacologically targeted.

Autophagy and Heart Disease

Autophagy is an intracellular bulk degradation process for proteins and organelles. In the heart, autophagy is stimulated by myocardial ischemia. However, the causative role of autophagy in the survival of cardiac myocytes and the underlying signaling mechanisms are poorly understood. Glucose deprivation (GD), which mimics myocardial ischemia, induces autophagy in cultured cardiac myocytes. Survival of cardiac myocytes was decreased by 3-methyladenine, an inhibitor of autophagy, suggesting that autophagy is protective against GD in cardiac myocytes. GD-induced autophagy coincided with activation of AMP-activated protein kinase (AMPK) and inactivation of mTOR (mammalian target of rapamycin). Inhibition of AMPK by adenine 9-β-D-arabinofuranoside or dominant negative AMPK significantly reduced GD-induced autophagy, whereas stimulation of autophagy by rapamycin failed to cause an additive effect on GD-induced autophagy, suggesting that activation of AMPK and inhibition of mTOR mediate GD-induced autophagy. Autophagy was also induced by ischemia and further enhanced by reperfusion in the mouse heart, in vivo. Autophagy resulting from ischemia was accompanied by activation of AMPK and was inhibited by dominant negative AMPK. In contrast, autophagy during reperfusion was accompanied by upregulation of Beclin 1 but not by activation of AMPK. Induction of autophagy and cardiac injury during the reperfusion phase was significantly attenuated in beclin 1^{+/-} mice. These results suggest that, in the heart, ischemia stimulates autophagy through an AMPK-dependent mechanism, whereas ischemia/reperfusion stimulates autophagy through a Beclin 1-dependent but AMPK-independent mechanism. Furthermore, autophagy plays distinct roles during ischemia and reperfusion: autophagy may be protective during ischemia, whereas it may be detrimental during reperfusion (Matsui Y et al., 2007).

### SUMMARY OF THE INVENTION

The present invention provides for up- and down-regulation of cellular autophagy for use, for example, in treating various diseases and disorders. The invention results, in part, from the discovery that two proteins, ATG14L (previously and alternatively called “BISC”) and Rubicon (previously and alternatively called “BIRC”), bind to a Class III phophatidylinositol 3′-kinase (PI3K)/Vps34-Beclin 1 autophagic complex. ATG14L and Rubicon each regulate autophagic activity in an opposing manner. ATG14L can increase autophagic activity and PI3K/Vps34 kinase activity; and Rubicon can decrease autophagic activity and PI3K/Vps34 kinase activity. ATG14L and Rubicon can be used, for example, to increase or decrease autophagic activity, respectively, or to increase or decrease PI3K/Vps34 kinase activity, respectively, and in so doing, treat diseases and disorders, such as cancer, neurodegenerative disease, stroke, metabolic disease, and age-related disease.

The present invention provides an isolated polypeptide that has an amino acid sequence up to 99% or up to 95% identical to SEQ ID NO: 1, comprising at least one amino acid sequence selected from the group consisting of amino acids 75 to 95 of SEQ ID NO: 1, and amino acids 148 to 178 of SEQ ID NO: 1. In one non-limiting embodiment, the polypeptide comprises a sequence of amino acids 99 to 492 of SEQ ID NO:1.

The present invention provides an isolated polypeptide that has an amino acid sequence up to 99% or up to 95% identical to SEQ ID NO: 3, comprising at least one amino acid sequence selected from the group consisting of amino acids 75 to 95 of SEQ ID NO: 3, and amino acids 148 to 178 of SEQ ID NO: 3.

The present invention provides an isolated polypeptide that has an amino acid sequence up to 99% or up to 95% identical to SEQ ID NO: 2, comprising at least one sequence of amino acids 488 to 508 of SEQ ID NO: 2. In one non-limiting embodiment, the polypeptide comprises a sequence of amino acids 208 to 836 of SEQ ID NO: 2. In one non-limiting embodiment, the polypeptide comprises a sequence of amino acids 1 to 836 of SEQ ID NO: 2. In one non-limiting embodiment, the polypeptide comprises a sequence of amino acids 208 to 941 of SEQ ID NO: 2.

The present invention provides an isolated polypeptide that has an amino acid sequence up to 99% or up to 95% identical to SEQ ID NO: 4, comprising at least one sequence of amino acids 518 to 538 of SEQ ID NO: 4.

Further, the present invention provides a nucleic acid encoding an ATG14L or Rubicon polypeptide as described above. In one embodiment, the nucleic acid is comprised in an expression vector. In one embodiment, the expression vector is a recombinant construct. The present invention further provides a pharmaceutical composition which comprises a therapeutically effective amount of the expression vector and a pharmaceutically acceptable carrier.

The present invention further provides a pharmaceutical composition which comprises a therapeutically effective amount of a purified ATG14L protein or functional fragment thereof, or a purified Rubicon protein or functional fragment thereof, and a pharmaceutically acceptable carrier. A functional fragment of an ATG14L or Rubicon protein of this invention is one that agonizes or antagonizes activity of the native ATG14L or Rubicon protein. It is particularly contemplated that certain functional fragments of ATG14L and Rubicon will act as competitive inhibitors of the native proteins.

The present invention also provides an oligonucleotide which is greater than or equal to 10 and less than 50 base pairs in length and hybridizes under a physiological condition to an ATG14L nucleic acid coding sequence as set forth in SEQ ID NO: 5, or its complementary sequence and/or has a nucleotide sequence that has at least 95% sequence identity with SEQ ID NO: 5, or its complementary sequence. In addition, the present invention also provides an oligonucleotide which is greater than or equal to 10 and less than 50 base pairs in length and hybridizes under a physiological condition to an ATG14L nucleic acid coding sequence as set forth in SEQ ID NO: 6, or its complementary sequence and/or has a nucleotide sequence that has at least 95% sequence identity with SEQ ID NO: 6, or its complementary sequence. In one embodiment, the oligonucleotide is an RNAi oligonucleotide, in particular, a siRNA. In one non-limiting embodiment, the RNAi oligonucleotide has a nucleotide sequence as set forth in SEQ ID NO: 11. In another embodiment, the oligonucleotide is an antisense oligonucleotide.

The present invention also provides an oligonucleotide which is greater than or equal to 10 and less than 50 bass pairs in length and hybridizes under a physiological condition to a Rubicon nucleic acid coding sequence as set forth in SEQ ID NO: 7, or its complementary sequence and/or has a nucleotide sequence that has at least 95% sequence identity with SEQ ID NO: 7, or its complementary sequence. In addition, the present invention also provides an oligonucleotide which is greater than or equal to 10 and less than 50 base pairs in length and hybridizes under a physiological condition to a Rubicon nucleic acid coding sequence as set forth in SEQ ID NO: 8, or its complementary sequence, and/or has a nucleotide sequence that has at least 95% sequence identity with SEQ ID NO: 8, or its complementary sequence. In one embodiment, the oligonucleotide is an RNAi oligonucleotide, in particular, a siRNA. In one non-limiting embodiment, the RNAi oligonucleotide has a nucleotide sequence as set forth in SEQ ID NO: 12. In another embodiment, the oligonucleotide is an antisense oligonucleotide.

The present invention also provides both an isolated anti-ATG14L antibody or antigen binding portion thereof, and an isolated anti-Rubicon antibody or antigen binding portion thereof.

Furthermore, the present invention provides for an isolated nucleic acid molecule encoding the anti-ATG14L or anti-Rubicon antibody, an expression or therapeutic vector which comprises the nucleic acid molecule encoding the anti-ATG14L or anti-Rubicon antibody, and a host cell which comprises the expression or therapeutic vector that comprises the nucleic acid molecule encoding the anti-ATG14L or anti-Rubicon antibody for intracellular expression, i.e., expression in the cytoplasm of a cell (intrabody).

Also provided is a method of increasing autophagic activity in a cell. In one embodiment, the method of increasing autophagic activity in a cell comprises administering to the cell an effective amount of an agent that increases or enhances the biological activity of ATG14L. The agent that increases or enhances the biological activity of ATG14L includes, but is not limited to, ATG14L, a functional agonistic fragment thereof, an ATG14L polypeptide equivalent, an ATG14L mimetic compound, a therapeutic vector which comprises a nucleic acid molecule encoding ATG14L protein, a binding enhancer which enhances or prolongs the binding between ATG14L and a class III PI3K/Vps34-Beclin-1 autophagic complex, and a binding enhancer that enhances or prolongs the binding between ATG14L and Beclin 1.

In another embodiment, the method of increasing autophagic activity in a cell comprises administering to the cell an effective amount of an agent that decreases or inhibits the biological activity of Rubicon. The agent that decreases or inhibits the biological activity of Rubicon includes but it not limited to, a functional antagonistic fragment of Rubicon, an anti-Rubicon antibody or fragment thereof such as an intrabody, another agent which inhibits or blocks Rubicon biological activity, and a nucleic acid targeted to the Rubicon gene, such as an antisense nucleic acid, a DNA construct for expression of an antisense RNA, a ribozyme, a DNA construct for expression of a ribozyme, a DNAzyme, and an RNAi agent.

In addition, the present invention provides a method of decreasing autophagic activity in a cell. In one embodiment, the method of decreasing autophagic activity in a cell comprises administering to the cell an effective amount of an agent that increases or enhances the biological activity of Rubicon. The agent that increases or enhances the biological activity of Rubicon includes, but is not limited to, a Rubicon, a functional agonistic fragment thereof, a Rubicon polypeptide equivalent, a Rubicon mimetic compound, a therapeutic vector which comprises a nucleic acid molecule encoding Rubicon protein, and a binding enhancer that enhances or prolongs the binding between Rubicon and a class III PI3K/Vps34-Beclin 1 autophagic complex.

In another embodiment, the method of decreasing autophagic activity in a cell comprises administering to the cell an effective amount of an agent which decreases or inhibits the biological activity of ATG14L. The agent that decreases or inhibits the biological activity of ATG14L includes but is not limited to, a functional antagonistic fragment of ATG14L, an anti-ATG14L antibody or fragment thereof such as an intrabody, another agent which inhibits or blocks ATG14L biological activity, and a nucleic acid targeted to the ATG14L gene, such as an antisense nucleic acid, a DNA construct for expression of an antisense RNA, a ribozyme, a DNA construct for expression of a ribozyme, a DNAzyme, and an RNAi agent.

Also provided is a method of increasing class III PI3K/Vps34 kinase activity in a cell. In one embodiment, the method of increasing class III PI3K/Vps34 kinase activity comprises administering to the cell an effective amount of an agent which increases or enhances the biological activity of ATG14L as described above.

In another embodiment, the method of increasing class III PI3K/Vps34 kinase activity comprises administering to the cell an effective amount of an agent which decreases or inhibits the biological activity of Rubicon as described above.

The present invention also provides a method of decreasing class III PI3K/Vps34 kinase activity in a cell. In one embodiment, the method of decreasing class III PI3K/Vps34 kinase activity comprises administering to the cell an effective amount of an agent which increases or enhances the biological activity of Rubicon as described above.

In another embodiment, the method of decreasing class III PI3K/Vps34 kinase activity comprises administering to the cell an effective amount of an agent which decreases or inhibits the biological activity of ATG14L as described above.

Furthermore, the present invention provides a method of screening for a compound that binds to an ATG14L polypeptide, which method comprises providing an ATG14L polypeptide, contacting a candidate compound with the ATG14L polypeptide; and determining whether the candidate compound had bound to the ATG14L polypeptide. In various embodiments, the ATG14L polypeptide is selected from the group consisting of murine ATG14L protein having an amino acid sequence of SEQ ID NO: 1, a polypeptide having an amino acid sequence that has at least 95% identity with SEQ ID NO: 1, human ATG14L protein having an amino acid sequence of SEQ ID NO: 3, and a polypeptide having an amino acid sequence that has at least 95% identity with SEQ ID NO: 3.

The present invention also provides a method of screening for a compound that binds to Rubicon, which method comprises providing a Rubicon polypeptide, contacting a candidate compound with the Rubicon polypeptide; and determining whether the candidate compound binds to the Rubicon polypeptide. In various embodiments, the Rubicon polypeptide is selected from the group consisting of murine Rubicon protein having an amino acid sequence of SEQ ID NO: 2, a polypeptide having an amino acid sequence that has at least 95% identity with SEQ ID NO: 2, human Rubicon protein having an amino acid sequence of SEQ ID NO: 4, and a polypeptide having an amino acid sequence that has at least 95% identity with SEQ ID NO: 4.

The present invention also provides a transgenic knock-out mouse which comprises a disruption in the endogenous ATG14L gene. In one non-limiting embodiment, the disruption can be introduced by homologous recombination of the endogenous ATG14L gene. In another non-limiting embodiment, the disruption can be made by introducing a gene encoding RNAi targeted to an ATG14L gene. The disruption of the ATG14L gene results in decreased expression of ATG14L protein.

The present invention also provides a transgenic knock-out mouse which comprises a disruption in the endogenous Rubicon gene. In one non-limiting embodiment, the disruption can be introduced by homologous recombination of the endogenous Rubicon gene. In another non-limiting embodiment, the disruption can be made by introducing a gene encoding RNAi targeted to a Rubicon gene. The disruption of the Rubicon gene results in decreased expression of Rubicon protein.

The present invention also provides a host cell which comprises an expression vector which comprises nucleic acid encoding ATG14L protein or a functional fragment thereof. In addition, the present invention provides a host cell which comprises an expression vector which comprises nucleic acid encoding Rubicon protein or a functional fragment thereof.

The present invention further provides a functional Beclin 1 fusion protein which comprises a tag for detecting the fusion protein, and a mature Beclin 1 having an amino acid sequence set forth in SEQ ID NO: 9 (human Beclin 1) (GenBank Accession No. NP_{—}003757) or SEQ ID NO: 10 (murine Beclin 1) (GenBank Accession No. NP_{—}062530), or an amino acid sequence that has at least 95% sequence identity with SEQ ID NO: 9 or SEQ ID NO: 10 and which retains Beclin 1 activity. In one embodiment, the activity of the Beclin 1 fusion protein is reduced by no more than 10% compared to the mature Beclin 1. In one embodiment, the tag is a green fluorescent protein (GFP). Such a Beclin 1 fusion protein is useful for screening for agents that enhance or inhibit biological activity of ATG14L or Rubicon.

The present invention also provides an isolated nucleic acid molecule encoding the functional Beclin 1 fusion protein as described above. The nucleic acid may optionally be comprised in an expression vector. Furthermore, the present invention provides for a host cell or a transgenic non-human animal which comprises an expression vector encoding the Beclin 1 fusion protein. The host cells include, but are not limited to, a bacterial cell, an eukaryotic cell, a yeast cell, a mammalian cell.

The present invention provides a method of screening for a compound that modulates biological activity of ATG14L, which method comprises (a) contacting ATG14L with a candidate compound in a manner wherein the candidate compound interacts with ATG14L; (b) evaluating the biological activity of ATG14L; and (c) identifying a candidate compound as a modulator of biological activity of ATG14L based upon observing increased or decreased biological activity of ATG14L in the presence of the candidate compound compared to a control sample.

The present invention also provides a method of screening for a compound that modulates biological activity of Rubicon, which method comprises (a) contacting Rubicon with a candidate compound in a manner wherein the candidate compound interacts with Rubicon; (b) evaluating the biological activity of Rubicon; and (c) identifying a candidate compound as a modulator of biological activity of Rubicon based upon observing increased or decreased biological activity of Rubicon in the presence of the candidate compound compared to a control sample.

The present invention further provides a method of treating cancer in a subject. In one embodiment, the method of treating cancer comprises administering to the subject a therapeutically effective amount of an agent which increases or enhances the biological activity of ATG14L, as described above. In another embodiment, the method of treating cancer comprises administering to the subject a therapeutically effective amount of an agent which decreases or inhibits the biological activity of Rubicon, as described above. The cancer includes, but is not limited to a cancer associated with an autophagy defect, such as breast cancer, liver cancer, or lung cancer.

Also provided is a method of treating a neurodegenerative disease in a subject. In one embodiment, the method of treating a neurodegenerative disease comprises administering to the subject a therapeutically effective amount of an agent which increases or enhances the biological activity of ATG14L, as described above. In another embodiment, the method of treating a neurodegenerative disease comprises administering to the subject a therapeutically effective amount of an agent which decreases or inhibits the biological activity of Rubicon, as described above. The neurodegenerative disease may be, for example, but not by way of limitation, a neurodegenerative disease associated with an autophagy defect, Parkinson's disease, Huntington's disease, Alzheimer's disease, or Amyotrophic Lateral Sclerosis (ALS).

Furthermore, the present invention provides a method of treating an inflammatory disease in a subject. In one embodiment, the method of treating an inflammatory disease comprises administering to the subject a therapeutically effective amount of an agent which increases or enhances the biological activity of ATG14L, as described above. In another embodiment, the method of treating an inflammatory disease comprises administering to the subject a therapeutically effective amount of an agent which decreases or inhibits the biological activity Rubicon, as described above. The inflammatory disease includes, but is not limited to, an inflammatory disease associated with an autophagy defect, and Crohn's disease.

The present invention also provides a method of treating an age-related disease in a subject. In one embodiment, the method of treating an age-related disease comprises administering to the subject a therapeutically effective amount of an agent which increases or enhances the biological activity of ATG14L, as described above. In another embodiment, the method of treating an age-related disease comprises administering to the subject a therapeutically effective amount of an agent which decreases or inhibits the biological activity of Rubicon, as described above. The age-related disease includes, but is not limited to an age-related disease associated with an autophagy defect.

Also provided is a method of treating heart disease in a subject. In one embodiment, the method comprises administering to the subject a therapeutically effective amount of an agent which modulates the biological activity of ATG14L. In one embodiment, the agent increases or enhances the biological activity of ATG14L as described above, during ischemia. In another embodiment, the agent decreases or inhibits the biological activity of ATG14L as described above, during reperfusion. In another embodiment, the method of treating heart diseases in a subject comprises administering to the subject a therapeutically effective amount of an agent which modulates the biological activity of Rubicon. In one embodiment, the agent decreases or inhibits the biological activity of Rubicon as described above, during ischemia. In another embodiment, the agent increases or enhances the biological activity of Rubicon as described above, during reperfusion.

The present invention provides a method of evaluating the expression of ATG14L in a cell in a biological sample, which method comprises detecting an ATG14L polypeptide in the biological sample. In one non-limiting embodiment, the method comprises (a) contacting the biological sample with an anti-ATG14L antibody or antigen binding portion thereof and (b) detecting the presence of an anti-ATG14L antibody or the antigen binding portion thereof that is specifically bound to the ATG polypeptide from the biological sample. The methods include, but are not limited to, Enzyme-Linked ImmunoSorbent Assay (ELISA), a Western blot, labeling the ATG14L polypeptide and identifying the labeled ATG14L polypeptide, a mass spectrometry, a gel electrophoresis, and a combination thereof.

The present invention also provides a method for evaluating expression of ATG14L in a cell in a biological sample, which method comprises detecting ATG14L mRNA in the biological sample. The methods include, but are not limited to a reverse transcription-polymerase chain reaction, Northern blotting, microarray, or a combination thereof.

In one non-limiting embodiment, the expression of ATG14L correlates to autophagic activity. For example, increased expression of ATG14L indicates increased autophagic activity, and decreased expression of ATG14L indicates decreased autophagic activity. In another non-limiting embodiment, the expression of ATG14L correlates to class III PI3K/Vps34 kinase activity. For example, increased expression of ATG14L indicates increased lass III PI3K/Vps34 kinase activity, and decreased expression of ATG14L indicates decreased lass III PI3K/Vps34 kinase activity.

The present invention provides a method of evaluating the expression of Rubicon in a cell in a biological sample, which method comprises detecting a Rubicon polypeptide in the biological sample. In one non-limiting embodiment, the method comprises (a) contacting the biological sample with an anti-Rubicon antibody or antigen binding portion thereof and (b) detecting the presence of an anti-Rubicon antibody or the antigen binding portion thereof that is specifically bound to the Rubicon polypeptide from the biological sample. The methods include, but are not limited to, Enzyme-Linked ImmunoSorbent Assay (ELISA), a Western blot, labeling the Rubicon polypeptide and identifying the labeled Rubicon polypeptide, a mass spectrometry, a gel electrophoresis, and a combination thereof.

The present invention also provides a method for evaluating expression of Rubicon in a cell in a biological sample, which method comprises detecting Rubicon mRNA in the biological sample. The methods include, but are not limited to a reverse transcription-polymerase chain reaction, Northern blotting, microarray, or a combination thereof.

In one non-limiting embodiment, the expression of Rubicon correlates to autophagic activity. For example, increased expression of Rubicon indicates decreased autophagic activity, and decreased expression of Rubicon indicates increased autophagic activity. In another non-limiting embodiment, the expression of Rubicon correlates to class III PI3K/Vps34 kinase activity. For example, increased expression of Rubicon indicates decreased lass III PI3K/Vps34 kinase activity, and decreased expression of Rubicon indicates increased lass III PI3K/Vps34 kinase activity.

The present invention provides a method of increasing the recruitment of ATG12, ATG5, and ATG6 to a class III PI3K/Vps34-Beclin-1 autophagic complex in a cell. The method comprises administering an effective amount of agent to the cell which increases or enhances the biological activity of ATG14L as described above.

The present invention also provides a method of decreasing the recruitment of ATG12, ATG5, and ATG6 to a PI3K/Vps34-Beclin-1 autophagic complex in a cell. The method comprises administering to the cell an effective amount of agent which decreases or inhibits the biological activity of ATG14L as described above.

Furthermore, the present inventions provides for a strategy that combines mouse genetics and a biochemical approach to purify Beclin 1-Vps34 complex from mouse tissues. The preliminary data have suggested that a large quantity of this complex can be obtained from a few of mice, which provides feasibility of a large-scale screening. Lipid kinase assay is developed by adapting the established and commercially available assay kits to Beclin 1-Vps34 kinase. Since the assay is targeted at the kinase activity in a native protein complex which exists under physiological condition, it provides a better opportunity for identifying highly efficient and biologically relevant chemicals that will work in vivo. The screening of different libraries is performed to identify kinase inhibitors (autophagy inhibitors) and enhancer (autophagy enhancer).

Also provided is an image-based high-throughput assay for ATG14L-Beclin 1 complex formation. A cell line stably expressing both Beclin 1-EGFP and ATG14L-mRFP at endogenous levels (by BAC transgenic expression which replace endogenous wild type Beclin 1 and ATG14L) is engineered. This cell line is tested with known autophagy inducers for its application in the assay. The autophagy inducer is expected to cause yellow punctate fluorescence (merge of red and green), indicative of ATG14L-Beclin 1 complex formation which results in autophagy activation. A chemical library is screened for the compounds that induce the fluorescent puncta formation. The identified chemicals are potential autophagy enhancers, and are further validated in the established autophagy assays. Alternatively, structure-based design of small chemicals that modulate Beclin 1-ATG14L interactions is performed.

The present invention also provides for an image-based high-throughput assay for Rubicon structure formation. A cell line stably expressing Rubicon-EGFP at endogenous levels by BAC transgenics is engineered. Formation of Rubicon-EGFP puncta in cytoplasm, as a result of distinct Rubicon complex formation lacking Beclin 1, is expected to cause autophagy inhibition. A chemical library is screened for compounds that promote the formation of Rubicon-EGFP puncta formation. The identified chemicals are potential autophagy inhibitors, and are further validated in the established autophagy assays. Furthermore, the structure of Rubicon C-terminal cystein-rich domain, which is responsible for Rubicon-EGFP puncta formation is studied. structure-based design of small chemicals that will disrupt the association of Rubicon with autophagy inhibition and thus enhancing autophagic activity is performed.

Also provided is in vitro and/or in vivo testing of leads. In testing the identified compounds in autophagy, a series of reporter cells and established transgenic mice are used. In testing the function of the leads, protein aggregate assay in cells is performed. In this assay, autophagy enhancer is expected to increase the degradation and prevent aggregate formation in cells and animal models (e.g. Huntington Disease mouse models). In addition, ischemia mouse models in heart or brain are used for autophagy inhibitors, which are expected to reduce the damage to the heart or brain.

These and other embodiments of the present invention are described in greater detail below, and in the Examples.

### BRIEF DESCRIPTION OF THE DRAWINGS

FIG. 1A-E. Identification of novel Beclin 1-interaction proteins from beclin 1^{-/-} beclin 1-EGFP/+ mice. (A) Western blot analysis showed the replacement of endogenous Beclin 1 with Beclin 1-EGFP in beclin 1^{-/-}; beclin 1-EGFP/+ mice, as detected by anti-Beclin 1 antibody. (B) Coomassie-stained SDS-PAGE gel revealed the Beclin 1-interacting proteins immuno-isolated from the brain and liver tissues of the “rescued” mice by using anti-GFP antibody (Lane 2 and 4). Protein bands in the gel were isolated and identified by mass spectrometric analysis as Vps15/p150 (band 1), Vps34/PtdIns3K (band 3), UVRAG (band 4), Beclin 1-EGFP (band 5) and two novel proteins ATG14L (band 6, asterisk, gi|27369860) and Rubicon (band 2, asterisk, gi|45708948). The control mice were the beclin 1^{+/-} littermates (P16) (Lane 1 and 3). (C) Schematic diagrams of the domain structures of ATG14L and Rubicon. ATG14L contains two coiled-coil domains (CCD1 and CCD2), which is also homologous to SMC domain (Structural Maintenance of Chromosomes). Rubicon contains an N-terminal RUN (for RPIP8, UNC-14 and NESCA) domain, a C-terminal cysteine-rich domain, and a CCD domain in the central of the protein sequence. (D and E) HEK 293T cells were transfected with either N-terminal FLAG-tagged (E) or C-terminal EGFP-tagged (E) ATG14L or Rubicon. Immunoprecipitation was performed with anti-FLAG (D) or anti-GFP antibody (E). The endogenous Beclin 1 co-immunoprecipitated were detected by Western blot analysis using anti-Beclin 1 antibody. WCL represents whole cell lysate, and IP represents immunoprecipitated.

FIG. 2A-E. Mapping of the binding domains that mediate the interaction between Beclin 1 and ATG14L/Rubicon. (A) Schematic representations of Beclin 1, ATG14L and Rubicon domain structures and the construction of multiple deletion mutants. The Beclin 1 mutants were tagged with FLAG at N-termini. The ATG14L or Rubicon mutants were tagged with EGFP at C-termini. (B) and (C) Domain mapping in Beclin 1 sequence required for binding ATG14L or Rubicon. HEK 293T cells were co-transfected with FLAG-tagged Beclin 1 mutant constructs and GFP-tagged full-length ATG14L (C) or Rubicon (D). The immunoprecipitation was performed with anti-GFP antibody, followed by Western blotting with anti-FLAG antibody. The results of these experiments were summarized in the Table 1.

TABLE 1

| | Beclin 1 domains | | | | |
|---|---|---|---|---|---|
| | B (Bcl2- | BC (Bcl2-binding | C (Coiled- | CE (Coiled-coil domain | |
| | binding | domain and Coiled- | coil | and Evolutionarily | E (Evolutionarily |
| | domain) | coil domain) | domain) | conserved domain) | conserved domain) |
| ATG14L | − | + | + | + (strong) | − |
| Rubicon | − | − | − | + | − |

“+” or “−” indicated positive or negative interactions between the tested protein domains. These experiments showed that coiled-coil and evolutionarily conserved domains of Beclin 1 (FLAG-CE) synergistically bound to ATG14L; only FLAG-CE of Beclin 1, not other Beclin 1 mutants, bound to Rubicon. “B” represents “Bcl2-binding domain,” “C” represents “Coiled-coil domain,” and “E” represents “Evolutionarily conserved domain.” (E) and (F) Domain mapping in ATG14L or Rubicon sequence required for binding Beclin 1. HEK 293T cells were transfected with EGFP-tagged ATG14L (E) or Rubicon (F) mutant constructs. The immunoprecipitation was performed with anti-GFP antibody, followed by Western blot analysis of endogenous Beclin 1 with anti-Beclin 1 antibody. The results of these experiments were summarized in Table 2.

TABLE 2

| | ATG14L mutants | | | Rubicon mutants | | | |
|---|---|---|---|---|---|---|---|
| | ΔCCD1 | ΔCCD2 | full length | ΔRUN | ΔC | ΔRUN&C | full length |
| Beclin 1 | + | − | +(strong) | + | + | + | +(weak) |

“+” or “−” indicated positive or negative interactions between the tested protein domains. These experiments showed that both CCD1 and CCD2 were important for ATG14L binding to Beclin 1, and that RUN or Cys-rich domain of Rubicon was not required for Rubicon binding to Beclin 1. Thus, central region containing CCD is important for binding. In addition, ATG14L mutants lacking RUN, Cys-rich domain or both domains have increased binding to Beclin1. Abbreviations: RUN—RUN domain, Cys—cysteine-rich domain.

FIG. 3A-C. Analysis of protein composition in the Beclin 1 complex(es). (A) Western blot analysis of Beclin 1, Vps34, ATG14L and Rubicon in the gel filtration fractions from wild type mouse liver extract. The peak levels of each protein were found co-eluded in the same fractions (Stenmark H. et al., FEBS Letters (2002); 513, 77 (2002); Odorizzi G. et al., Trends in Biochemical Sciences (2000); 25, 229). ATG14L was also found eluted at the second peak levels in later fractions. The cell lysate of FLAG-ATG14L-transfected HEK 293T was loaded as a positive control (labeled with “1”) for the migration position of the ATG14L protein. The adjacent lane (labeled with “2”) was loaded with cell lysates without transfection. (B) and (C) Co-immunoprecipitation experiments showing protein-protein interaction between ATG14L and Rubicon. HEK 293 cells were co-transfected with EGFP-tagged ATG14L and FLAG-tagged Rubicon (B), or EGFP-tagged Rubicon and FLAG-tagged ATG14L (C). Cell lysates were used for immunoprecipitation with anti-GFP antibody, and the resulting immunoprecipitates were blotted with anti-FLAG antibody. The result showed that EGFP-tagged ATG14L was able to pull down FLAG-tagged Rubicon (B), and vice versa (C). “WCL” represents “whole cell lysate;” and “IP” represents immunoprecipirated.

FIG. 4A-E. ATG14L functions in positive regulation of autophagy. (A) Beclin 1 or ATG14L RNAi knock-down in the NIH 3T3 cells led to increased levels of p62/SQSTM1 and LC3 II form as detected with anti-p62/SQSTM1 and LC3 antibodies under both normal (left panel) and nutrient-starvation conditions (right panel). Notably, Beclin 1 RNAi knock-down markedly reduced levels of ATG14L expression. β-actin was blotted as the loading control. (B) Confocal images of MLE12 cells stably expressing GFP-LC3 showed that ATG14L RNAi knock-down resulted in decreased number of small size GFP-LC3 puncta, but markedly increased number of large size GFP-LC3 puncta, as compared to the control RNAi treatment. Scale bar, 10 μm. (C) Confocal images of MLE12 cells stably expressing GFP-LC3 showed that ATG14L-RNAi-induced large-size GFP-LC3 puncta (in green) colocalized with p62/SQSTM1 (in red). Scale bar, 10 μm. (D) Confocal images of MLE12 cells stably expressing GFP-LC3 showed that ATG14L-RNAi-induced large size GFP-LC3 puncta (in green) co-localized with ubiquitin (in red). Scale bar, 10 μm. (E) Vps34/PtdIns-3 kinase activity assay. HEK 293T cells were co-transfected with Myc-Vps34-Vps15 and FLAG-tagged ATG14L or empty vector, either in the absence or in the presence of Beclin 1-EGFP. Myc-tagged Vps34 that was immunoprecipitated using anti-Myc antibody were used for the kinase assay. The resulting radioactive PtdIns(3)P was separated by thin layer chromatography and visualized by radiograph. The amount of radioactive PtdIns(3)P was normalized against the amount of immunoprecipitated Myc-tagged Vps34. The result were quantified and showed that in the presence of Beclin 1, over-expressed ATG14L significantly up-regulated Vps34 kinase activity (ratio=2.5, p=0.04 for the one-tailed Student's t-test with unequal variances, n=5, labeled by asterisk).

FIG. 5A-I. Synergistic effects of Beclin 1 and ATG14L in promoting double membrane formation. (A) Beclin 1-EGFP or ATG14L-EGFP is diffuse in cytoplasm of HEK-293T cells stably transfected with Beclin 1-EGFP or ATG14L-EGFP plasmids. Scale bar: 10 μm. (B) Co-expression and co-localization of ATG14L-EGFP (green) & Beclin 1-AsRed (red) in a number of punctate structures in transfected HeLa cells, with Pearson's coefficient (PC) 0.91. Scale bar: 10 μm. (C) Electron microscopic images showed that co-transfection with ATG14L-EGFP and Beclin 1-AsRed caused the formation of large size organelles (asterisks) that were often enwrapped with double membranes in HEK 293T cells: (C1) concentric membrane “rings”; (C2) two large structures (3-5 μm in diameter) contain materials with high electron density. The inset shows high magnification of enwrapping double membranes; (C3) formation of many autophagosomes (arrows) in cytoplasm; (C4) Immuno-electron microscopic image showing a structure enwrapped with concentric membrane “rings” labeled with anti-GFP antibody. “M” represents mitochondria, “N” represents nucleus. Scale bar: 500 nm. (D) Co-localization of Beclin 1-Myc (blue), ATG14L-AsRed (red) and GFP-LC3 (green) in the puncta. HeLa cells stably expressing GFP-LC3 were transiently transfected with Beclin 1-Myc and ATG14L-AsRed. Confocal imaging was performed to examine their localization. Scale bar: 10 μm. (E) and (F) Atg12-EGFP (green) or Atg5-EGFP was colocalized with the puncta associated with the ATG14L-AsRed (red) and Beclin 1-Myc (blue) (arrows) in transfected HeLa cells. Some of these puncta (arrows captured in boxes) appeared to be “ring” shape. Scale bar: 10 μm. (G) and (H) The ATG 14L-Beclin 1-positive structures in the HeLa cells transiently transfected with ATG14L-EGFP (in green) and Beclin 1-AsRed (in red) were not labeled by the Golgi marker syntaxin 6 (G) or the ER marker protein disulphide isomerase (PDI) (H) (in blue). Scale bars, 10 μm. (I) Partial colocalization of Rubicon/Rubicon-EGFP and EEA1 in the HEK 293 cells that were stably expressing Rubicon-EGFP and immunostained with the antibody against Rubicon (pseudocolored in green, actual experiment was performed with Alexa Fluo 555) and EEA1 (pseudocolored in red). Scale bar, 10 μm.

FIG. 6A-E. Rubicon is a negative regulator of autophagy. (A) Rubicon RNAi knock-down in the NIH 3T3 cells led to decreased levels of p62 and LC3 II as detected with anti-p62 and LC3 antibodies under both normal and nutrient-starvation conditions. (B) Over-expression of Rubicon resulted in increased levels of p62 under both normal and nutrient-starved conditions in both HEK-293T cells stably expressing Rubicon-EGFP (upper panel) and HEK-293T cells transiently transfected with Rubicon-EGFP (lower panel). The control cells were transfected with EGFP-N3 vector. (C) Vps34/PtdIns3K kinase activity. HEK 293T cells were co-transfected with Myc-Vps34-Vps15 and FLAG-tagged Rubicon or empty vector, either in the absence or in the presence of Beclin 1-EGFP. Myc-tagged Vps34 that was immunoprecipitated using anti-Myc antibody were used for the kinase assay. The resulting radioactive PtdIns(3)P was separated by thin layer chromatography and visualized by radiograph. The amount of radioactive PtdIns(3)P was normalized against the amount of immunoprecipitated Myc-tagged Vps34. The results were quantified and showed that in the absence of Beclin 1, over-expressed Rubicon significantly reduced Vps34 kinase activity (ratio=0.58, p=0.04 for the one-tailed Student's t-test with unequal variances, labeled by asterisk). (D) HeLa cells were transiently co-transfected with mCherry-GFP-LC3 and FLAG or FLAG-Rubicon. Confocal images show that cells co-expressing FLAG vector and mCherry-GFP-LC3 contain many red-only puncta along with yellow puncta (merge of red and green) (upper panel). In contrast, cells co-expressing FLAG-Rubicon and mCherry-GFP-LC3 contain primarily yellow puncta (lower panel, lower arrows). Noticeably, in FLAG-Rubicon and mCherry-GFP-LC3 co-transfection experiment, some cells express high levels of mCherry-GFP-LC3 and low levels of FLAG-Rubicon. These cells contain many red-only puncta (low panel, upper arrows), similar to the observation in mCherry-GFP-LC3 and FLAG transfected cells. This study indicates over-expression of Rubicon blocks acidification of mCherry-GFP-LC3 or autophagosome maturation. (E) Quantization of the result in (D). The result showed that over-expressing FLAG-Rubicon drastically reduced percentage of red-only puncta (mCherry-LC3) from 39% in the control cells to 2% in the FLAG-Rubicon-transfected cells (p=2E-26 for the one-tailed Student's t-test with unequal variances, n=30 for each case, labeled by asterisk).

FIG. 7A-D. Overexpressing Rubicon causes aberrant expansion of late endosomes/lysosomes. (A) Colocalization of Rubicon-EGFP-associated structures with the late endosome/lysosome marker Lamp1 (arrows) in HeLa cells transfected with Rubicon-EGFP. Note that some of the Rubicon-EGFP-associated structures showed a ‘ring’ shape (arrows marked with *). Scale bar, 10 μm. (B) Partial colocalization of Rubicon-EGFP-associated structures with the MVB marker LBPA (arrows) in HeLa cells transfected with Rubicon-EGFP. Scale bar, 10 μm. (C) Representative ultrastructural images show aberrant expansion of late endosomal/lysosomal structures in HEK 293T cells overexpressing Rubicon-EGFP. These abnormal organelles are large in size, with high (arrows marked with **) or low (black arrows) electron density. Some enclose small vesicles (arrows marked with t) and some resemble the MVB (arrows marked with ‡). Scale bars, 500 nm. (D) Representative ultrastructural images show late endosome/lysosome-like structures that are labelled with anti-GFP gold particles (panels 3, 4) in HEK 293T cells transiently transfected with Rubicon-EGFP. These structures are enwrapped by double membranes (panel 4 inset) and co-labelled by anti-GFP (developed by DAB) and anti-Lamp1 (gold enhanced) (panels 5-7) antibodies. Note that mitochondria are mostly negative for Rubicon-EGFP (panel 4). The negative controls are without antibody (panels 1-2). “M” represents mitochondria. Scale bars, 200 nm

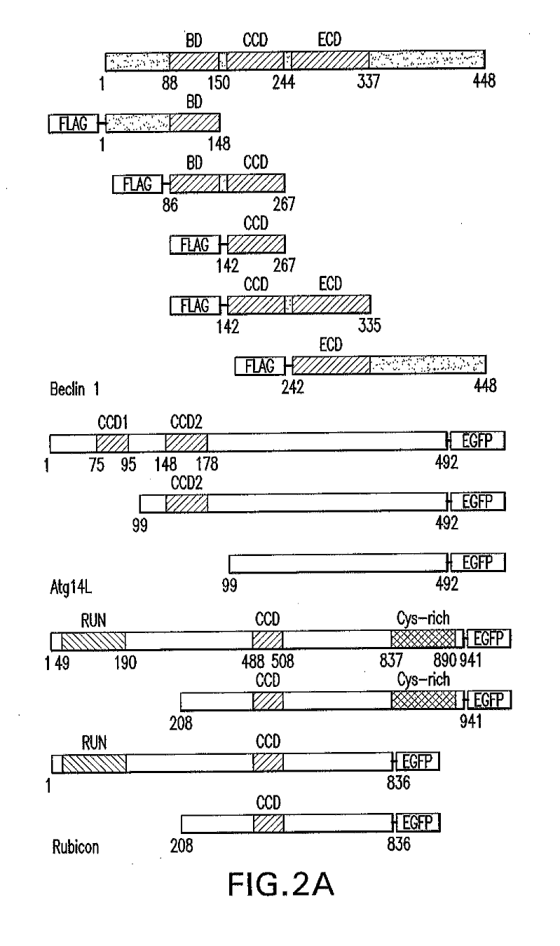

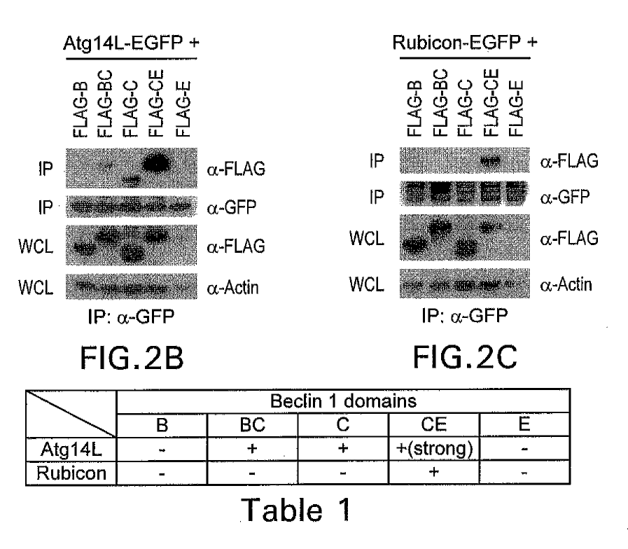

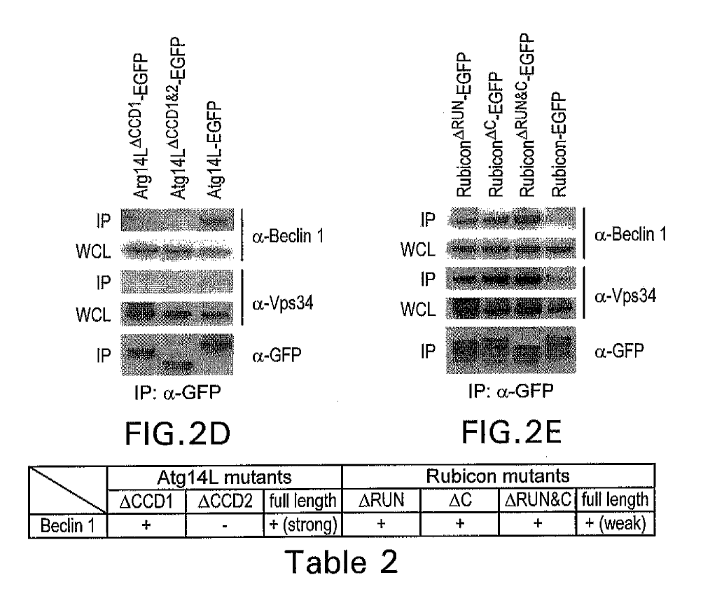

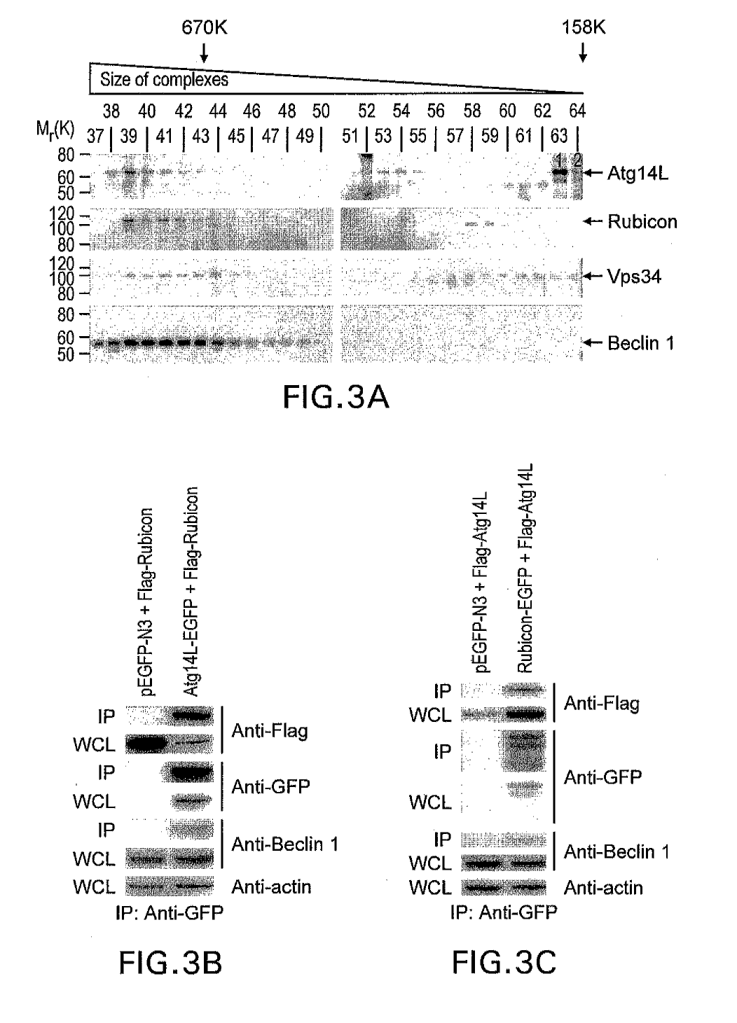

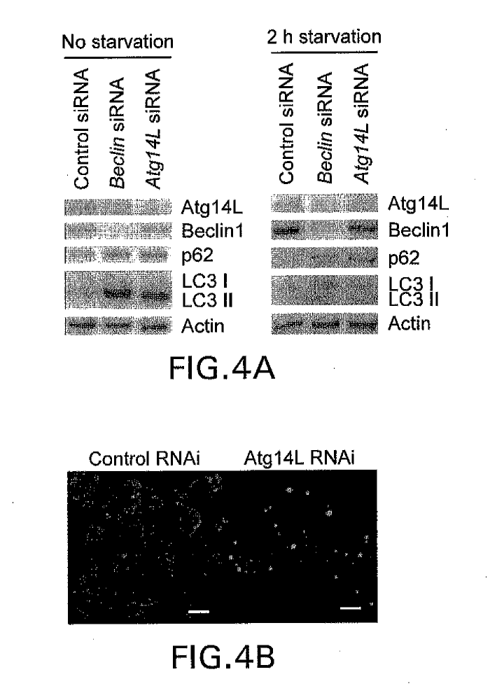

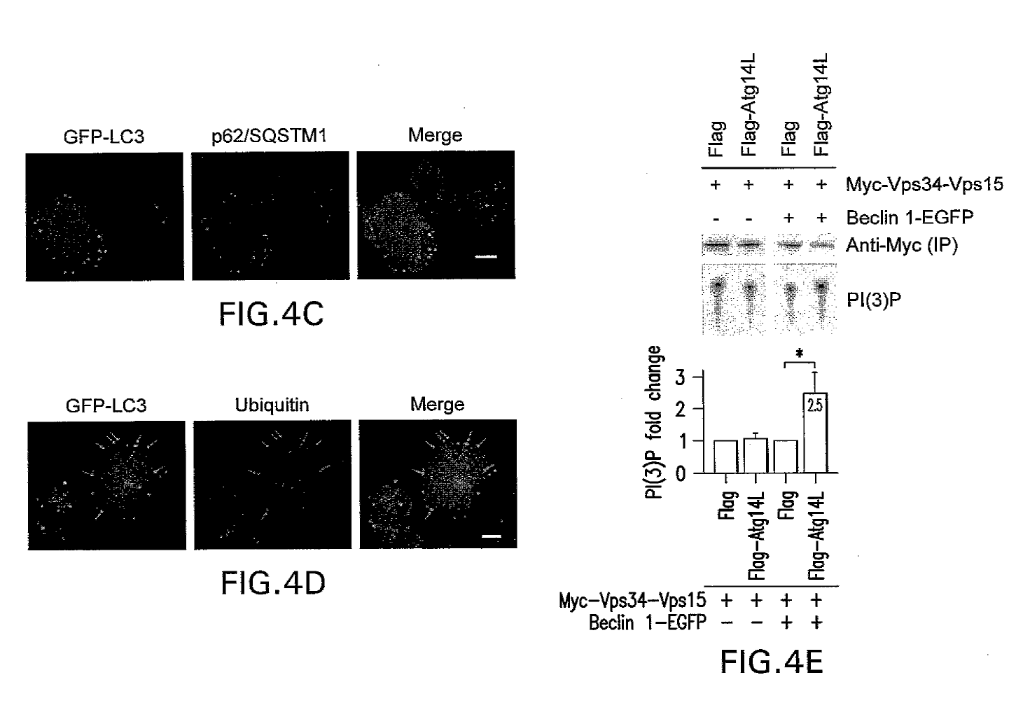

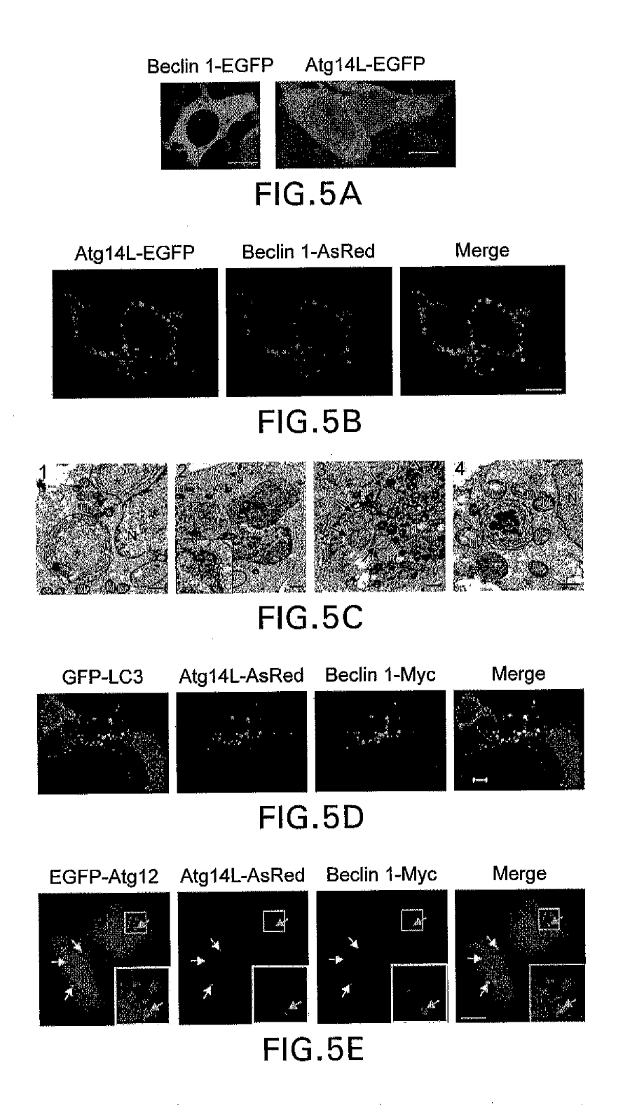

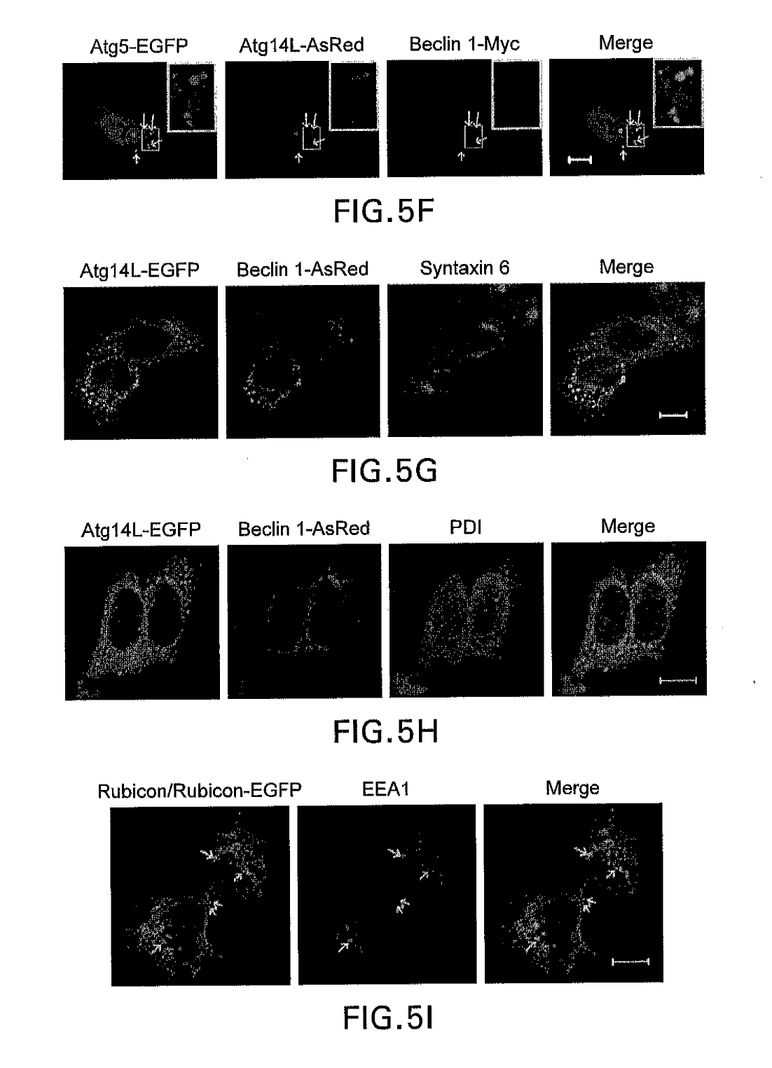

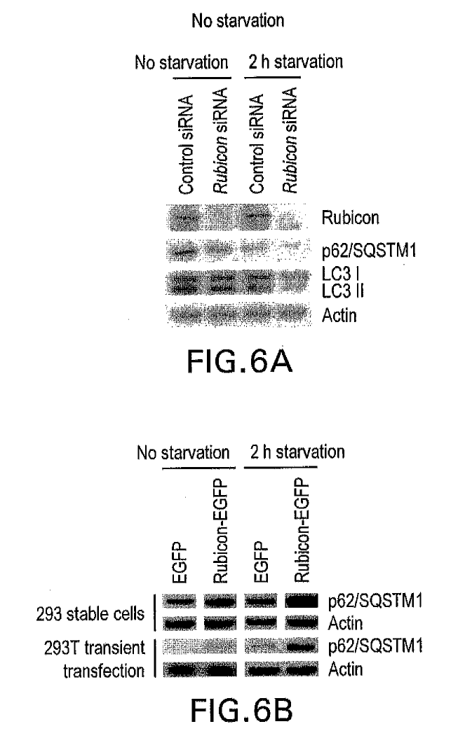

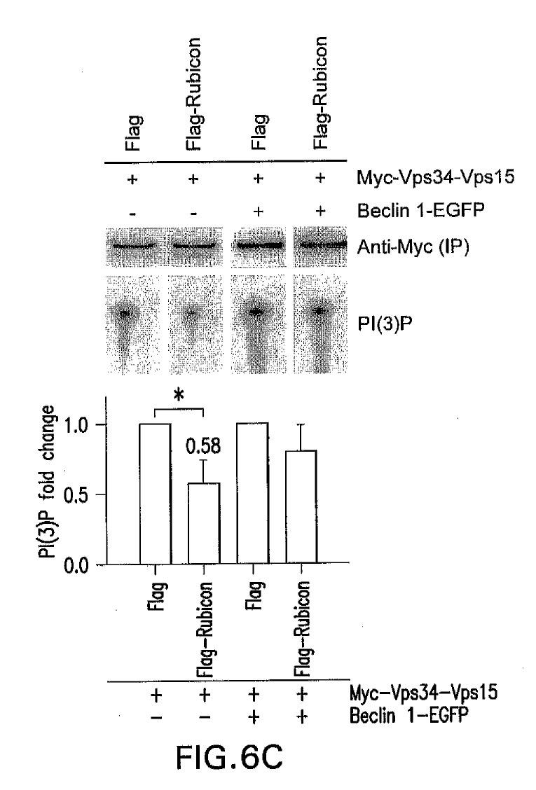

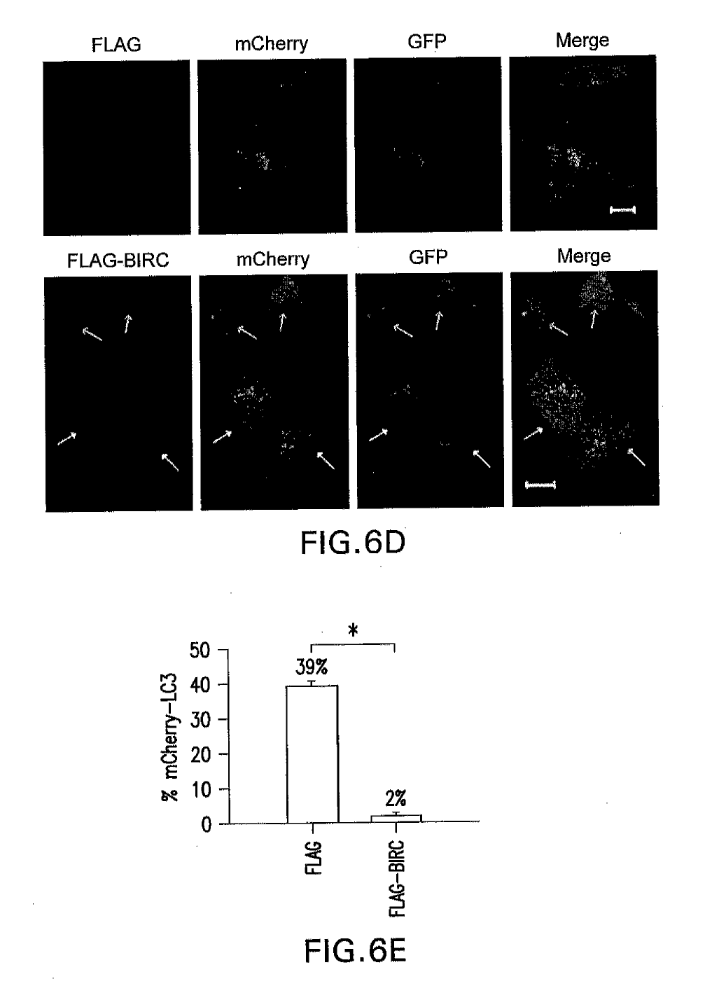

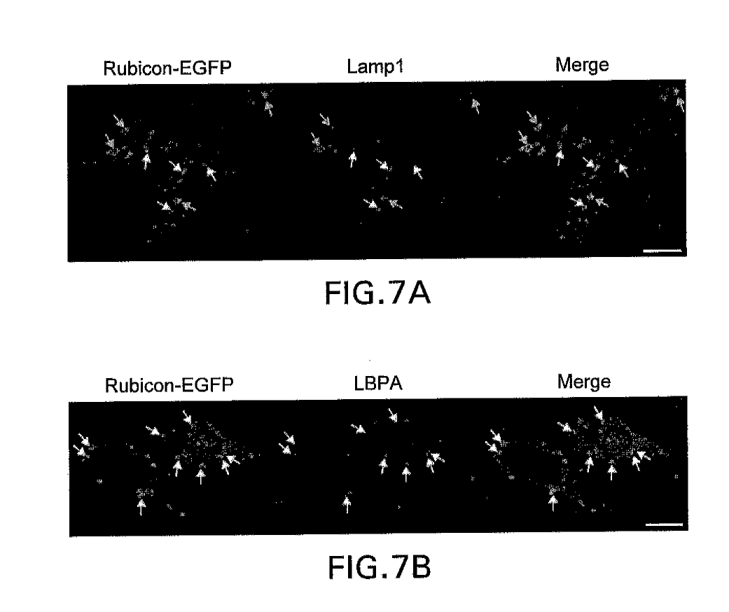

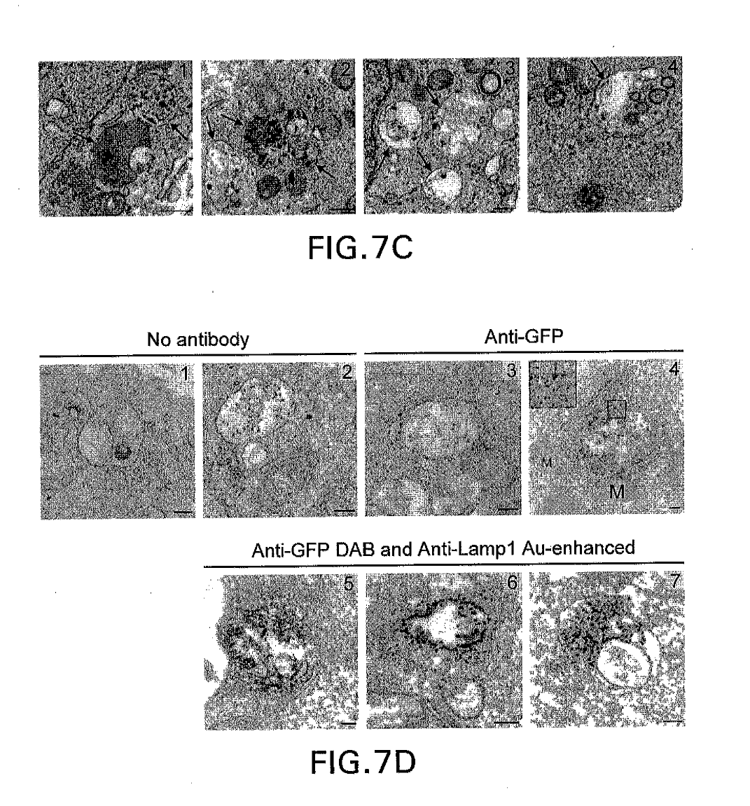

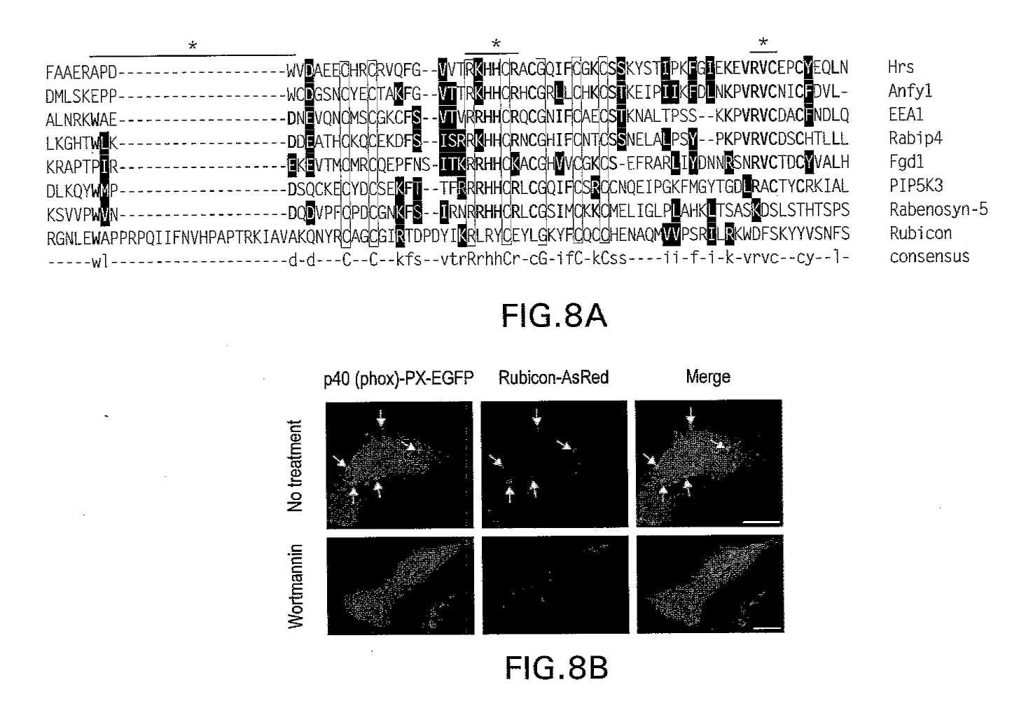

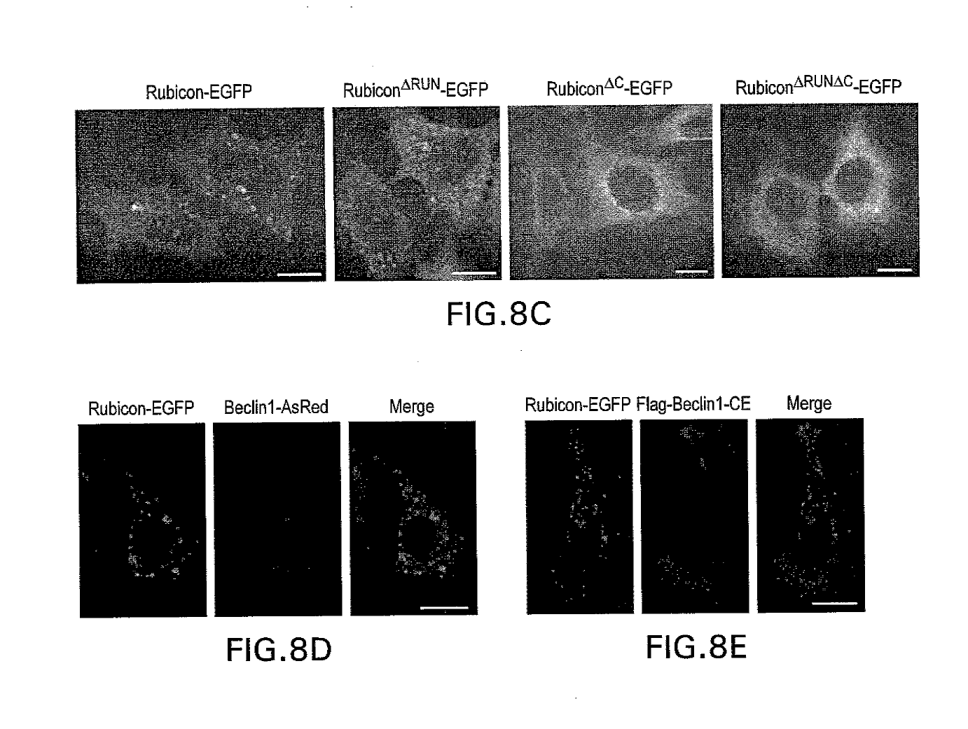

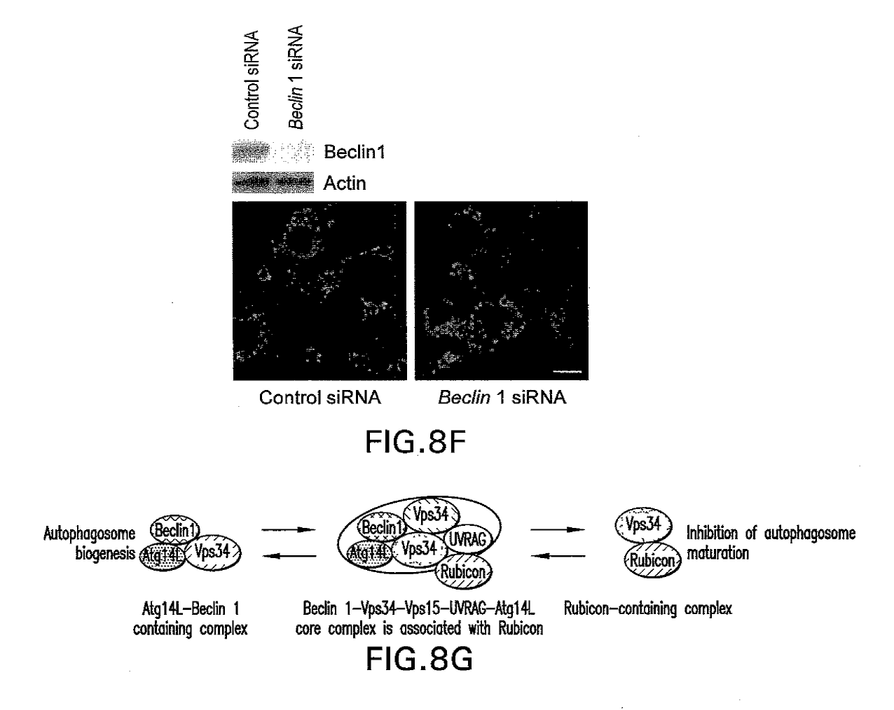

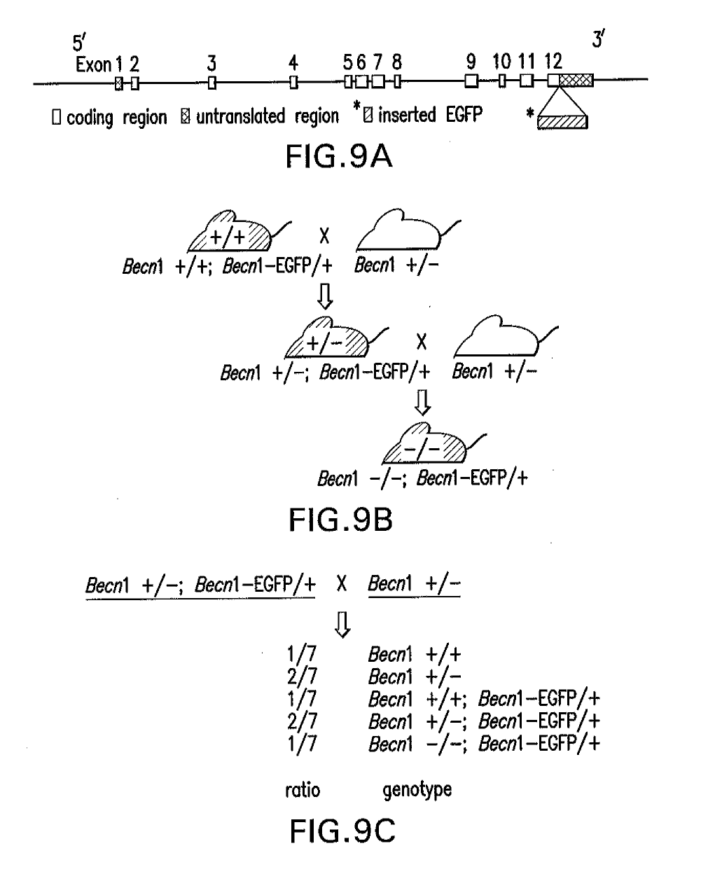

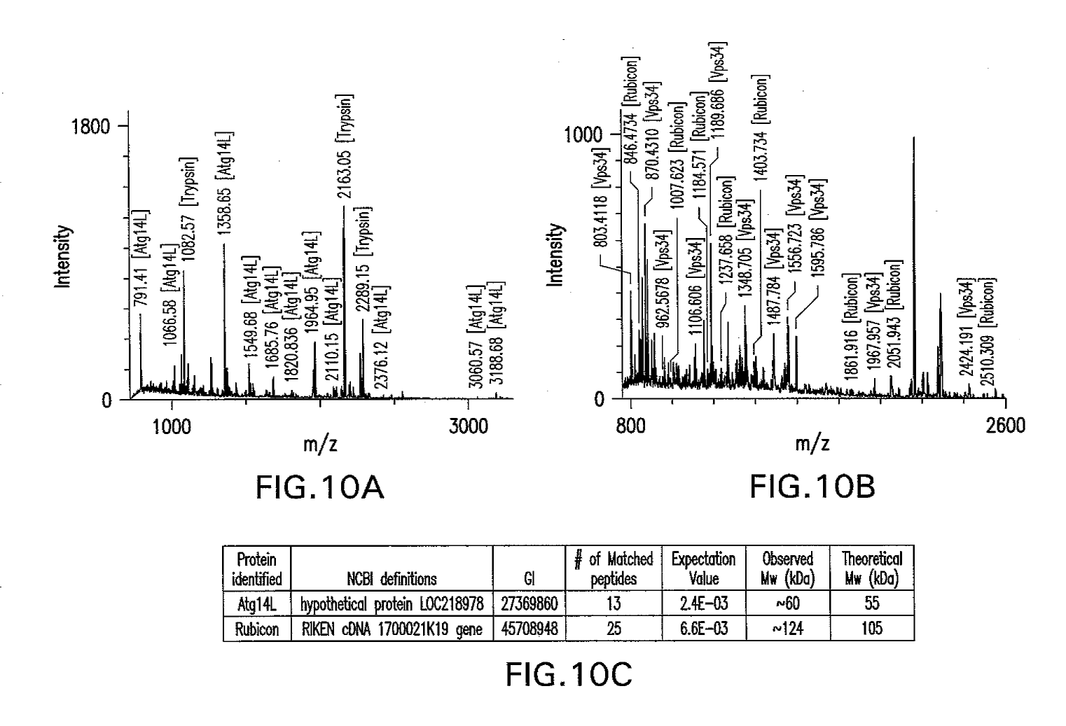

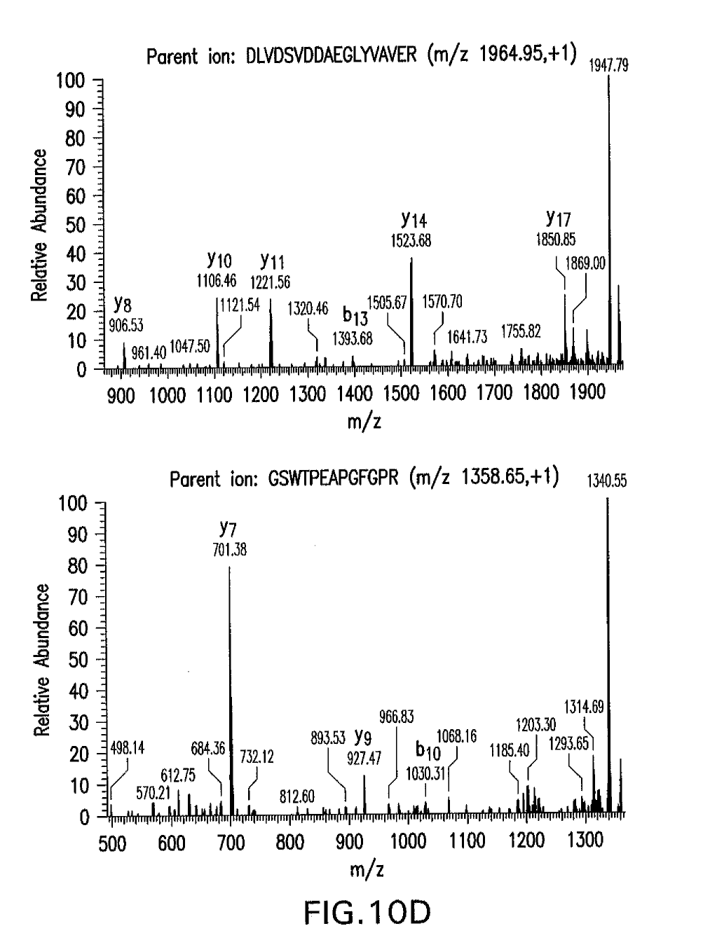

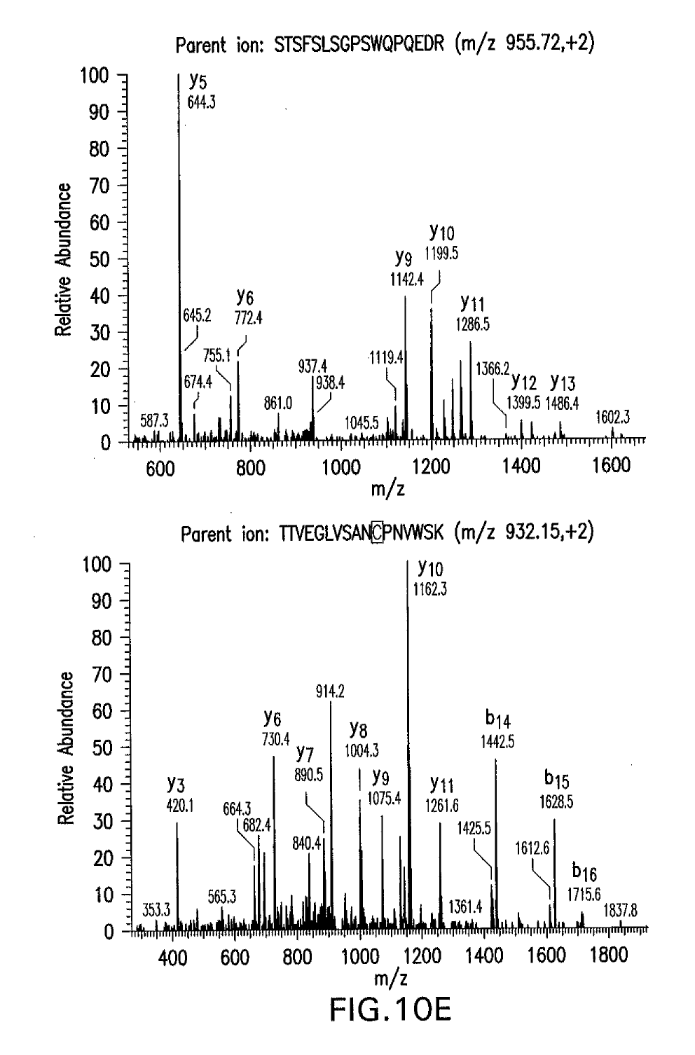

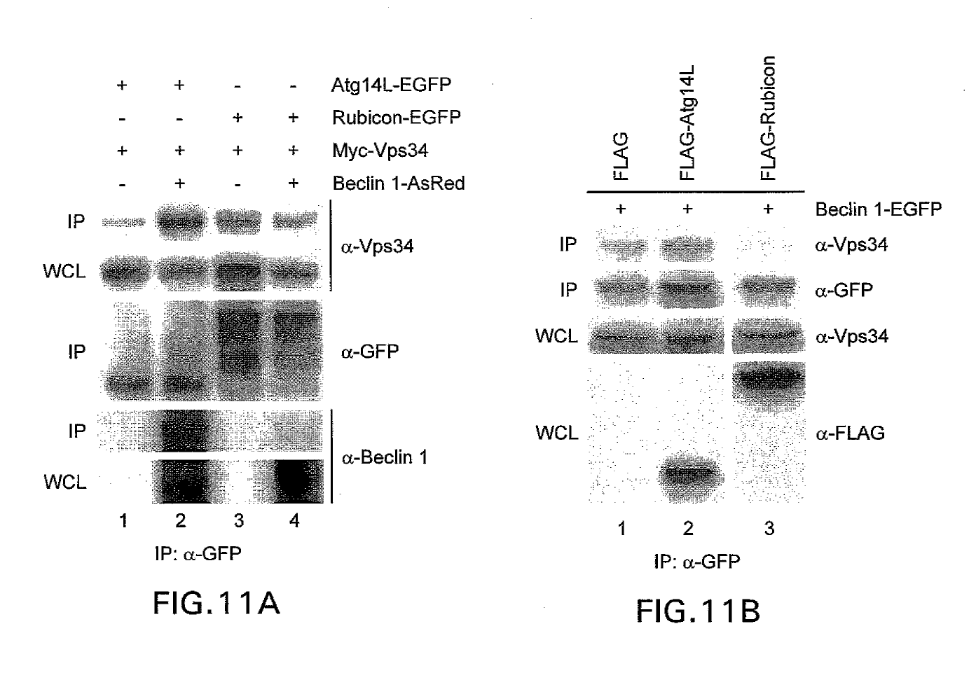

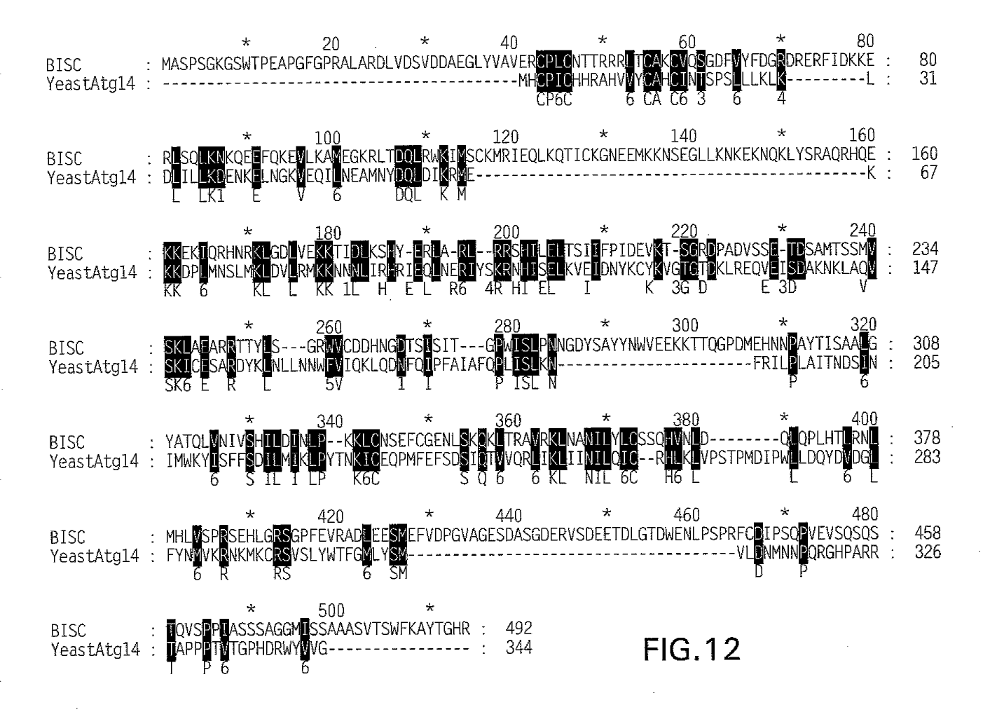

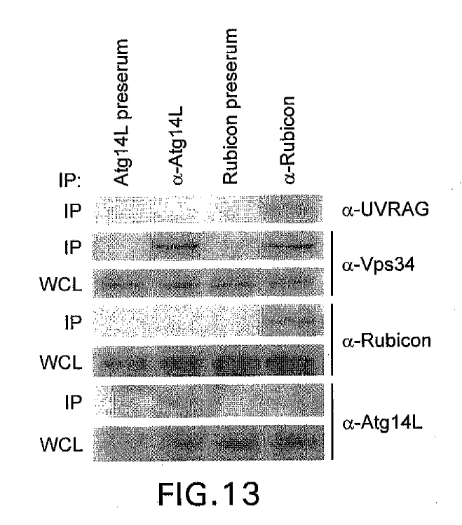

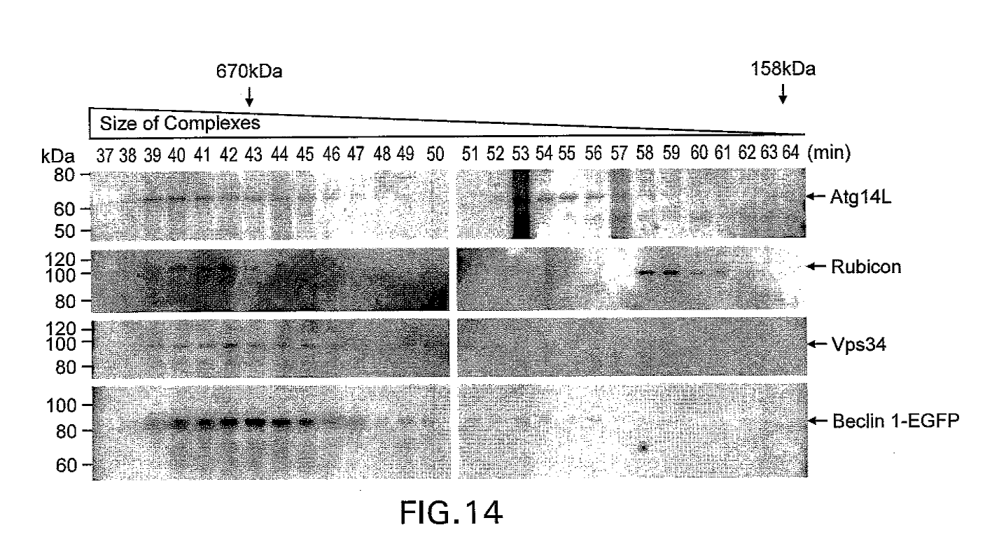

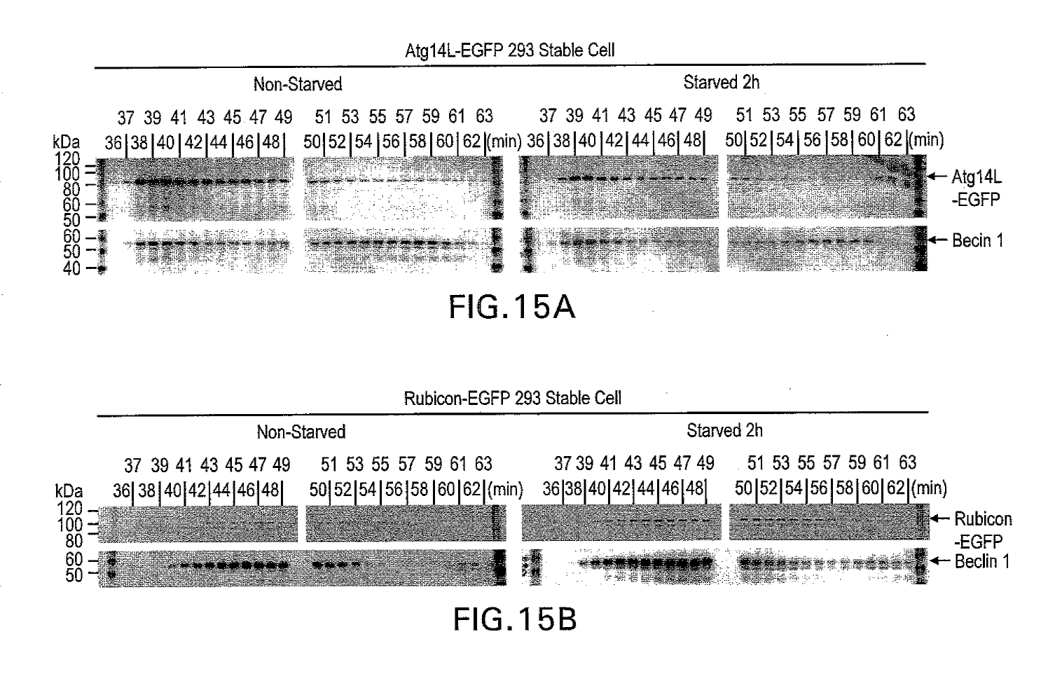

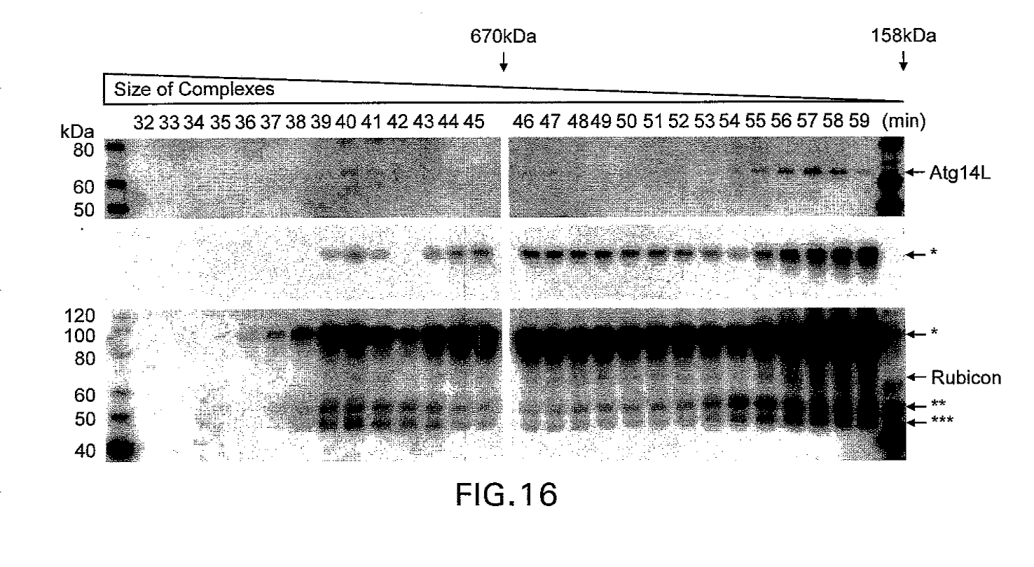

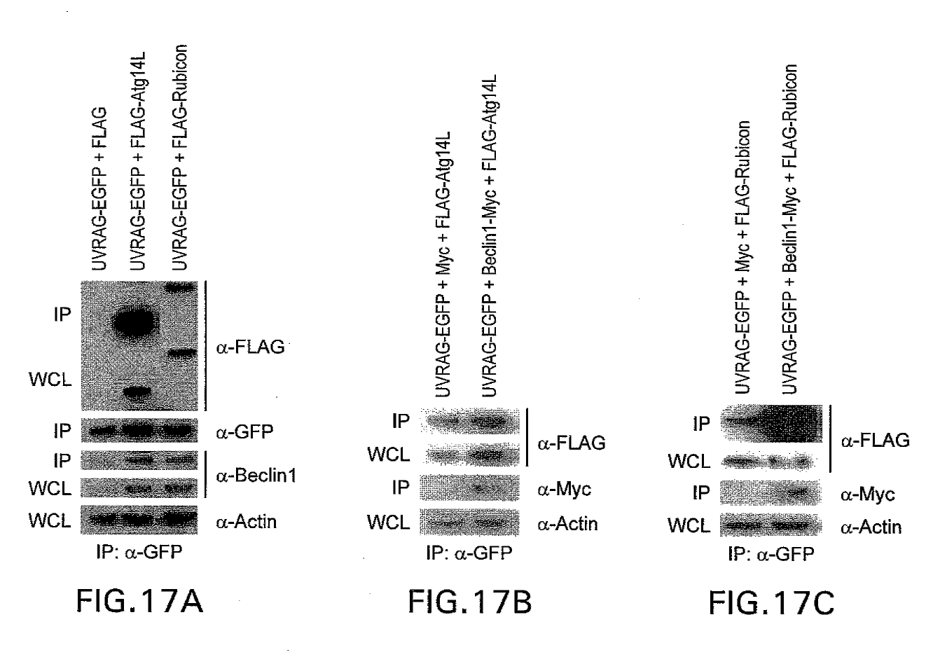

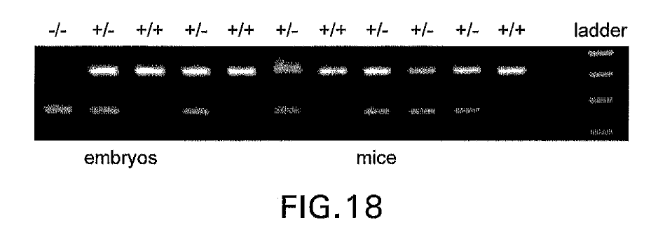

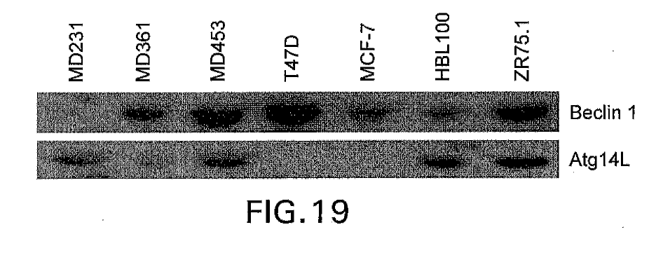

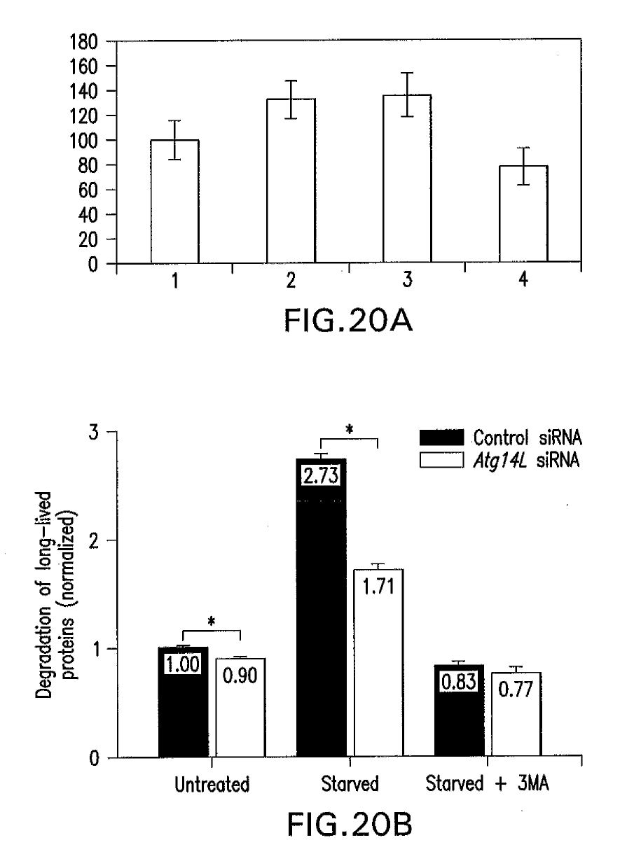

FIG. 8A-G. Over-expression of Rubicon is localized in PtdIns(3)P enriched-structures but independent of Beclin 1. (A) Local sequence alignment between the C-terminal cystein-rich domain in Rubicon and FYVE domains in several known FYVE-containing proteins. Rubicon does not possess the key consensus sequences of typical FYVE domain (indicated by red bars), i.e., N-terminal WxxD, central R[R/K]HHCR and C-terminal RVC. (B) p40 (phox)-PX-EGFP (green) and Rubicon-AsRed (red) were colocalized on large punctate structures (white arrows) in HeLa cells, suggesting localization of Rubicon on the PtdIns(3)P-enriched lipid membranes (upper panels). The formation of Rubicon-AsRed-positive structures (red) was insensitive to the treatment of Vps34/PtdIns3K inhibitor, wortmannin (75 nM for 1 h), whereas PtdIns(3)P-enriched lipid domains as marked by p40 (phox)-PX-EGFP (green) disappeared upon such treatment (lower panels). HeLa cells were co-transfected with p40 (phox)-PX-EGFP and Rubicon-AsRed. Cell imaging was performed with cofocal laser scanner microscope. Scale bar: 10 μm. (C) Subcellular distribution of ATG14L-EGFP full length and truncated mutants of ATG14L-EGFP. HeLa cells were transfected with ATG14L-EGFP, mutants Rubicon(ΔRUN)-EGFP, Rubicon(AC)-EGFP and Rubicon(ΔRUNΔC)-EGFP. Note that Rubicon(ΔC)-EGFP and Rubicon(ΔRUNΔC)-EGFP did not show punctate localization, in contrast to ATG14L-EGFP and Rubicon(ΔRUN)-EGFP. Scale bar: 10 (D) and (E) Absence of full-length Beclin 1 (D) or Beclin 1-CE mutant (E) (red) on Rubicon-EGFP-positive structures (green) in the HEK 293 cells stably expressing Rubicon-EGFP. These cells were transiently transfected with either (D) Beclin 1-AsRed or (E) FLAG-tagged Beclin 1 mutant containing coiled-coil and evolutionary conserved domains (FLAG-Beclin 1-CE), which was shown to mediate interaction between Beclin 1 and Rubicon (FIG. 2). Scale bars, 10 μm. (F) The formation of Rubicon-EGFP-positive structures was not affected by Beclin 1 RNAi knock-down in HEK 293 cells stably expressing Rubicon-EGFP. The knock down of Beclin 1 was assessed by Western blot using anti-Beclin 1 antibody. (G) A hypothetic model for Beclin 1-Vps34/PtdIns3K protein complexes and their functions. Under physiological condition, a core Beclin 1 complex is composed of Vps34/PtdIns3K, p150 (Vps15), Beclin 1 and UVRAG. A large Beclin 1-Vps34/PtdIns3K complex was formed when the core complex was associated with two additional proteins ATG14L and Rubicon in vivo. This large complex may be reduced to form small protein complexes such as Beclin 1-ATG14L-containing complex and Rubicon-containing complex. These small complexes are likely the functional units participating in autophagy regulation by modulating and directing Vps34/PtdIns3K lipid kinase activity.

FIG. 9A-C. Beclin 1-EGFP transgene rescues the embryonic lethality of beclin 1 homozygous deletion (beclin 1^{-/-}). (A) Schematic representation of EGFP insertion in BAC-mediated Beclin I-EGFP transgenics. (B) Breeding strategy for generating mice in which beclin 1-EGFP transgene replaces the endogenous beclin 1 alleles. Beclin 1-EGFP/+ mice were crossed with beclin 1^{+/-} mice to generate “rescued mice” (beclin 1^{-/-}; beclin 1-EGFP/+). (C) Theoretical distribution (Mendeline ratio) of each mouse genotype in the progenies of the indicated breeding pairs. From this breeding, the theoretical fraction of the mouse progenies with the beclin 1^{-/-}; beclin 1-EGFP/+ is 1/7. In these experiments, 11 out of a total of 69 mouse progenies (from eight litters) derived from above cross were the beclin 1^{-/-}; beclin 1-EGFP/+ phenotype, supporting that Beclin 1-EGFP completely rescued the early embryonic lethality in beclin 1^{-/-} mice, and thus can functionally substitute for the endogenous Beclin 1 protein.

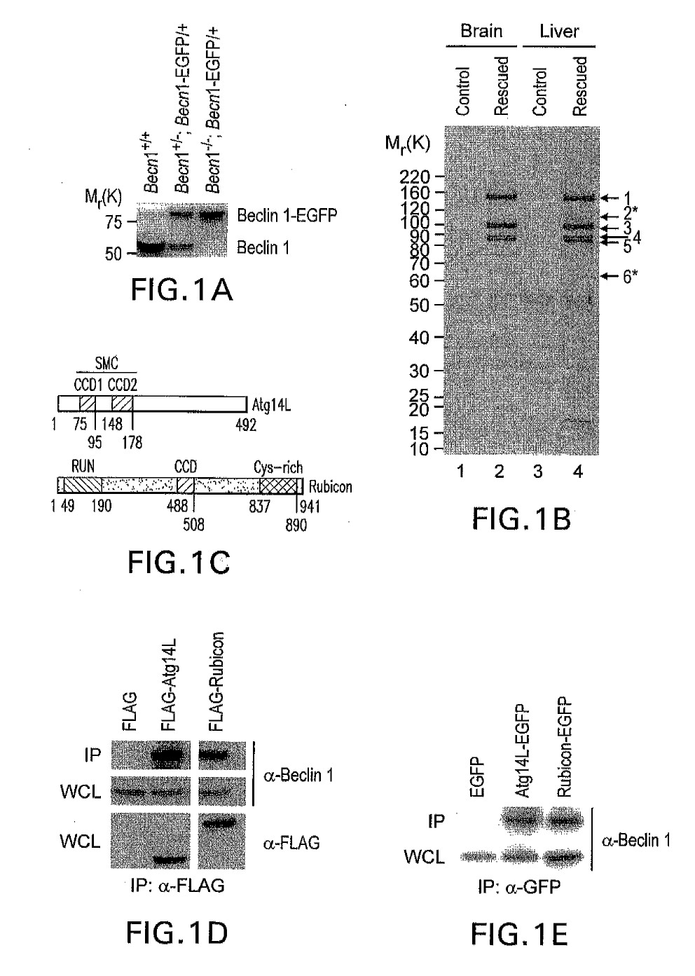

FIG. 10A-E: Identification of ATG14L and Rubicon by mass spectrometry. (A) Mass spectrum of ATG14L identified from the gel band 6 as shown in FIG. 1B. The sequence coverage was 35.5%. (B) Mass spectrum of ATG14L identified from the gel band 2 as shown in FIG. 1(b). The sequence coverage was 32.0%. In both (A) and (B), the gel band was excised, reduced and alkylated with iodoacetamide, and trypsin-digested before analyzed on a MALDI-QqTOF mass spectrometer. The resulting peptide masses were searched against the NCBI non-redundant database for Mus musculus using the search engine ProFound. (C) Summary of the peptide mapping results for ATG14L and Rubicon, and Table 3 is incorporated as below:

TABLE 3

| Protein | NCBI | | # of Matched | Expectation | Observed | Theoretical |
|---|---|---|---|---|---|---|
| identified | definitions | GI | peptides | Value | Mw (kDa) | Mw (kDa) |
| Atg14L | hypothetical | 27369860 | 13 | 2.4E−03 | ~60 | 55 |
| | protein LOC218978 | | | | | |
| Rubicon | RIKEN cDNA | 45708948 | 25 | 6.6E−03 | ~124 | 105 |
| | 1700021K19 gene | | | | | |

(D) Representative tandem mass spectra of ATG14L tryptic peptides, as identified by a MALDI ion trap mass spectrometer. (E) Representative tandem mass spectra of Rubicon tryptic peptides, as identified by a LC-MS (ESI linear ion trap) mass spectrometer. The cysteine in the second peptide contained a carbamidomethyl modification (in box). This particular sample was isolated from brains of “rescued” mice starved for 48 hours.

FIG. 11A-B. Binding of ATG14L or Rubicon to Beclin 1 and Vps34/PtdIns3K. (A) Co-immunoprecipitation of Beclin 1 and Vps34 with ATG14L-EGFP or Rubicon-EGFP. ATG14L-EGFP or Rubicon-EGFP was co-expressed with Myc-Vps34/PtdIns3K and either with or without Beclin 1-AsRed in HEK 293T cells. Immunoprecipitation was performed using anti-GFP antibody followed by blotting with anti-Vps34/PtdIns3K and anti-Beclin 1 antibodies. (B) Coimmunoprecipitation of endogenous Vps34 with Beclin 1-EGFP in the presence or absence of over-expressed FLAG-ATG14L or FLAG-Rubicon. Beclin 1-EGFP was co-expressed with FLAG-ATG14L, FLAG-Rubicon or empty pCMV-FLAG2 vector in the HEK 293T cells. Immunoprecipitation was performed with anti-GFP antibody, followed by blotting with anti-Vps34/PtdIns3K antibody to detect endogenous Vps34/PtdIns3K. Over-expression of FLAG-Rubicon , but not FLAG-ATG14L, repressed the amount of Vps34/PtdIns3K that interacted with Beclin 1-EGFP. For both (A) and (B), “WCL” represents “whole cell lysate;” and “IP” represents immunoprecipirated.

FIG. 12. Moderate homology between ATG14L and yeast Atg14. Protein sequences of ATG14L and yeast Atg14 were aligned to analyze possible homology between them. Alignment was done suing T-Coffee package (Tree based Consistency Objective Function For AlignmEnt Evaluation) available at server: www.tcoffee.org., and analysis was performed with GeneDoc application. The exact match between ATG14L and yeast Atg14 was 15%, and juxtaposition greater than zero was 27%. The numbers indicate similar groups: 1, DN; 2, EQ; 3, ST; 4, KR; 5, FYW; 6, LIVM.

FIG. 13. Analysis of protein interactions between endogenous UVRAG, ATG14L and Rubicon. Immunoprecipitation of endogenous ATG14L- and Rubicon-interacting proteins using anti-ATG14L and anti-Rubicon antibodies from NIH 3T3 cell lysates. The control experiments were performed using the corresponding preimmune serum for each antibody. The whole cell lysates (WCL) and immunoprecipitated samples (IP) were examined by Western blotting with antibodies against UVRAG, Vps34, ATG14L and Rubicon. “WCL” represents “whole cell lysate;” and “IP” represents immunoprecipirated.

FIG. 14. Western blot analysis of Beclin 1, Vps34, ATG14L and Rubicon in the gel filtration fractions from the “rescued” mice liver samples. The liver lysates were prepared from “rescued” mice beclin 1^{-/-}; beclin 1-GFP/+ and applied in gel filtration experiment. Western blot analysis of Beclin 1, Vps34, ATG14L and Rubicon in the gel filtration fractions is shown. The peak levels of each protein were found co-eluded in the same fractions (˜41-43). ATG14L was also found eluted at the second peak levels in later fractions. This result is consistent with the observation using wild-type mice (FIG. 3A).

FIG. 15A-B. Gel filtration analysis of protein distribution using stably transfected cells. (A) Western blot analysis of ATG14L-EGFP and Beclin 1 in the gel filtration fractions of non-starved (left panel) and starved (for 2 hours, right panel) using ATG14L-EGFP HEK 293 stable cell lysates. ATG14L-EGFP (recognized by anti-GFP antibody as a ca. 85 kDa band) and Beclin 1 co-eluted infractions around 39-40 minutes, supporting the specificity of the anti-ATG14L antibody (FIG. 3A). No change in the elution time was detected after the cells were starved for 2 h. (B) Western blot analysis of Rubicon-EGFP and Beclin 1 in the gel filtration fractions of non-starved (left panel) and starved (for 2 hours, right panel) using Rubicon-EGFP HEK 293 stable cell lysates. Rubicon-EGFP and Beclin 1 co-eluted in many fractions, supporting the specificity of the anti-Rubicon antibody (FIG. 3A). No change in the elution time was detected after the cells were starved for 2 hours.

FIG. 16. Gel filtration analysis of ATG14L-Rubicon interaction in the Beclin 1-Vps34/PtdIns3K complex(es) in vivo. The wild type mouse brain samples were treated either without (upper panel) or with anti-Rubicon antibody (middle and lower panels) before loading onto the size exclusion column. Western blots using anti-ATG14L antibody showed ˜65 kDa bands in the fractions from both samples. In addition, three major anti-Rubicon bands were also detected by the secondary anti-rabbit antibody (labeled by “*”, “**” and “***”). The co-elution of ATG14L and anti-Rubicon antibody suggested that ATG14L and Rubicon were in the same Beclin 1-Vps34/PtdIns3K complex. The fractions were labeled by their elution times. The UV trace of the gel filtration calibrants was also shown with the elution times when the peaks for 670 kDa and 158 kDa calibrants coming out designated by arrows. The lower panel is a film with long exposure for the sample treated with anti-Rubicon antibody before loading onto the size exclusion column. The middle panel is short exposure for the same blot (lower panel) in order to reveal the unsaturated intensity of the strongest antibody band labeled by an asterisk (“*”).

FIG. 17A-C. Protein interactions among Beclin 1, ATG14L, Rubicon and UVRAG assayed in transiently transfected cells. (A) Protein interaction between co-expressed UVRAG and ATG14L/Rubicon. HEK 293T cells were transfected with UVRAG-EGFP with FLAG-ATG14L or FLAG-Rubicon. Co-immunoprecipitation was performed with anti-GFP antibody, followed by detection with anti-FLAG antibody. The result showed that FLAG-ATG14L or FLAG-Rubicon was co-immunoprecipitated with UVRAG-EGFP. (B) and (C) Effect of Beclin 1 over-expression on protein interaction between co-expressed UVRAG and ATG14L/Rubicon. UVRAG-EGFP was co-expressed with either FLAG-ATG14L or FLAG-Rubicon in HEK 293T cells, in the absence or in the presence of Beclin 1-Myc. Co-immunoprecipitation was performed with anti-GFP antibody, followed by detection with anti-FLAG and anti-Myc antibodies. The results showed that Beclin 1 over-expression markedly increased the amount of FLAG-Rubicon that was co-immunoprecipitated with UVRAG-EGFP (C) while having little effect on interaction between FLAG-ATG14L and UVRAG-EGFP (B). “WCL” represents “whole cell lysate;” and “IP” represents immunoprecipitated.

FIG. 18. Genotyping of ATG14L knock-out mice and embryos. The results showed that heterozygous ATG14L^{+/-} mice were expected to develop tumors, and homozygous ATG14L^{-/-} was embryonic lethal.

FIG. 19. Detection of Beclin 1 and Atg14L in breast cancer cell lines by Western Blotting. The results showed lower or no expression of ATG14L in some of the breast cancer cell lines, for example, there was no expression of ATG14L in the T47D and MCF-7 cell dines, and low expression of ATG14L in the MD361 cell lines.

FIG. 20A-B. A. The effect of RNAi knock-down on cell number of 3T3 cell lines. “1” represents the native control RNAi; “2” represents Beclin 1 RNAi; “3” represents ATG14L RNAi; and “4” represents Rubicon RNAi. The results showed that knock-down of Beclin 1 and knock-down of ATG14L caused cell over-proliferation. Knock-down of Rubicon slowed down the cell growth. B. Compared with control siRNA, ATG14L siRNA decreased long-lived protein degradation in NIH 3T3 cells under normal (*p=0007) and starvation (*p=5×10⁻⁶) conditions (one tailed Student's T test with equal variances, n=4), this difference was diminished with the starved cells were treated with 3-methyladenine (3MA, 10 mM) a PI(3)K inhibitor.

### DETAILED DESCRIPTION OF THE INVENTION

The present invention provides agents and methods for modulating autophagic activity of mammalian cells. The invention is based, in part, on the experiments described in the Examples attached hereto.

In order that the present disclosure may be more readily understood, certain terms are defined below.

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

### ATG14L

ATG14L was previously referred to in U.S. Provisional Application No. 61/096,724 as “BISC.” The present invention pertains to various ATG14L polypeptides, nucleic acids and antibodies and their uses.

For example, various embodiments of the present invention pertain to methods for increasing autophagic activity, increasing ATG14L binding to Beclin 1, or increasing Class III PI3K/Vps34 kinase activity which comprise administering to a cell an effective amount of an agent which increases or enhances the biological activity of ATG14L. An agent that increases or enhances the biological activity of ATG14L includes, but is not limited to, ATG14L itself, a functional agonist fragment thereof, an ATG14L mimetic compound, a therapeutic vector which comprises a nucleic acid molecule encoding ATG14L protein, a binding enhancer which enhances or prolongs the binding between ATG14L and a class III PI3K/Vps34-Beclin 1 autophagic complex, and a binding enhancer which enhances or prolongs the binding between ATG14L and Beclin 1.

Other non-limiting embodiments of the invention pertain to methods of decreasing autophagic activity, decreasing binding of ATG14L to Beclin 1, or decreasing class III PI3K/Vps34 kinase activity in a cell which comprises administering to the cell an effective amount of an agent which decreases or inhibits the biological activity of ATG14L. An agent that decreases or inhibits the biological activity of ATG14L includes, but is not limited to, a functional antagonist fragment of ATG14L, an anti-ATG14L antibody or fragment thereof such as an intrabody, another agent which inhibits or blocks ATG14L biological activity, or a nucleic acid targeted to the ATG14L gene, such as an antisense nucleic acid, a DNA construct for expression of an antisense RNA, a ribozyme, a DNA construct for expression of a ribozyme, a DNAzyme, or an RNAi.

The full-length amino acid sequence of murine ATG14L (GenBank accession number: NP_{—}766187; gi|27369860) has 492 amino acids, which is provided below, and is designated SEQ ID NO: 1.

(SEQ ID NO: 1)
MASPSGKGSWTPEAPGFGPRALARDLVDSVDDAEGLYVAVERCPLCNTTRRR
LTCAKCVQSGDFVYFDGRDRERFIDKKERLSQLKNKQEEFQKEVLKAMEGKRLTDQLR
WKIMSCKMRIEQLKQTICKGNEEMKKNSEGLLKNKEKNQKLYSRAQRHQEKKEKIQRH
NRKLGDLVEKKTIDLKSHYERLARLRRSHILELTSIIFPIDEVKTSGRDPADVSSETDSAM
TSSMVSKLAEARRTTYLSGRWVCDDHNGDTSISITGPWISLPNNGDYSAYYNWVEEKK
TTQGPDMEHNNPAYTISAALGYATQLVNIVSHILDINLPKKLCNSEFCGENLSKQKLTRA
VRKLNANILYLCSSQHVNLDQLQPLHTLRNLMHLVSPRSEHLGRSGPFEVRADLEESME
FVDPGVAGESDASGDERVSDEETDLGTDWENLPSPRFCDIPSQPVEVSQSQSTQVSPPIAS
SSAGGMISSAAASVTSWFKAYTGHR

The full-length amino acid sequence of human ATG14L has 492 amino acids, which is provided below, and is designated SEQ ID NO: 3.

(SEQ ID NO: 3)
MASPSGKGARALEAPGCGPRPLARDLVDSVDDAEGLYVAVERCPLCNTTRRR
LTCAKCVQSGDFVYFDGRDRERFIDKKERLSRLKSKQEEFQKEVLKAMEGKWITDQLR
WKIMSCKMRIEQLKQTICKGNEEMEKNSEGLLKTKEKNQKLYSRAQRHQEKKEKIQRH
NRKLGDLVEKKTIDLRSHYERLANLRRSHILELTSVIFPIEEVKTGVRDPADVSSESDSAM
TSSTVSKLAEARRTTYLSGRWVCDDHNGDTSISITGPWISLPNNGDYSAYYSWVEEKKT
TQGPDMEQSNPAYTISAALCYATQLVNILSHILDVNLPKKLCNSEFCGENLSKQKFTRAV
KKLNANILYLCFSQHVNLDQLQPLHTLRNLMYLVSPSSEHLGRSGPFEVRADLEESMEF
VDPGVAGESDESGDERVSDEETDLGTDWENLPSPRFCDIPSQSVEVSQSQSTQASPPIASS
SAGGMISSAAASVTSWFKAYTGHR

ATG14L protein is predicted to comprise two coiled-coil domains (CCDs). The predicted CCDs of murine ATG14L protein are located at amino acids 75-95 and amino acids 148-178 of SEQ ID NO: 1, and the predicted CCDs of human ATG14L protein are located at amino acids 75-95 and amino acids 148-178 of SEQ ID NO: 3. Both CCDs of ATG14L are shown to be important for the efficient binding/interaction between ATG14L and Beclin 1.

Native murine ATG14L polypeptide is a consecutive amino acid segment of SEQ ID NO: 1, including the full length sequence of SEQ ID NO: 1. In non-limiting embodiments of the invention, an ATG14L polypeptide may comprise all or less than all of SEQ ID NO:1 (e.g., it may contain up to 100%, or up to 99%, or up to 95% sequence identity with the entirety of SEQ ID NO:1), and comprise a region that binds to Beclin 1 under physiologic conditions. In one non-limiting embodiment, the ATG14L polypeptide comprises a sequence of amino acids 99 to 492 of SEQ ID NO:1.

Native human ATG14L polypeptide is a consecutive amino acid segment of SEQ ID NO: 3, including the full length sequence of SEQ ID NO: 3. In non-limiting embodiments of the invention, an ATG14L polypeptide may comprise all or less than all of SEQ ID NO:3 (e.g., it may contain up to 100%, or up to 99%, or up to 95% sequence identity with the entirety of SEQ ID NO:3), and comprise a region that binds to Beclin 1 under physiologic conditions. In one embodiment, an ATG14L polypeptide having biological activity of a native ATG14L protein, the biological activity of a native ATG14L protein is as described in the examples, and exhibits properties including but not limited to, ability to bind to Beclin 1, to bind to a class III PI3K/Vps34-Beclin 1 autophagic complex, to increase class III PI3K/Vps34 kinase activity, and/or to increase autophagic activity.

The polypeptides of the present invention are preferably provided in an isolated form, and preferably are substantially purified. A recombinantly produced version of the ATG14L polypeptide can be substantially purified by the one-step method described at Smith and Johnson, Gene (1988); 67:31-40. Polypeptides of the invention can also be purified from natural or recombinant sources using anti-ATG14L antibodies of the invention in methods which are well known in the art of protein purification.

In non-limiting embodiments, the present invention provides an isolated polypeptide that has an amino acid sequence up to 99% or up to 95% identical to SEQ ID NO: 1 and comprises at least one amino acid sequence selected from the group consisting of amino acids 75 to 95 of SEQ ID NO: 1 and amino acids 148 to 178 of SEQ ID NO: 1. In one non-limiting embodiment, the polypeptide comprises a sequence of amino acids 99 to 492 of SEQ ID NO: 1. In another non-limiting embodiment, the present invention provides an isolated polypeptide that has an amino acid sequence up to 99% or up to 95% identical to SEQ ID NO: 3 and comprises at least one amino acid sequence selected from the group consisting of amino acids 75 to 95 of SEQ ID NO: 3, and amino acids 148 to 178 of SEQ ID NO: 3. In non-limiting embodiments, a polypeptide which is “up to 95% identical” or “up to 99” identical” to SEQ ID NO:1 is also greater than 50%, greater than 55%, greater than 60%, greater than 65%, greater than 70%, greater than 75%, greater than 80%, greater than 85%, or greater than 90% identical to SEQ ID NO: 1, and a polypeptide which is “up to 95% identical” or “up to 99” identical” to SEQ ID NO:3 is also greater than 50%, greater than 55%, greater than 60%, greater than 65%, greater than 70%, greater than 75%, greater than 80%, greater than 85%, or greater than 90% identical to SEQ ID NO:3. The polypeptides of the present invention also include polypeptides having an amino acid sequence at least 80% identical, more preferably at least 90% identical, and still more preferably 95%, 96%, 97%, 98% or 99% identical to those described above, as well as polypeptides having an amino acid sequence with at least 90% similarity, and more preferably at least 95% similarity, to those described above. Preferably, the aforementioned polypeptides are capable of binding to Beclin 1, of binding to a class III PI3K/Vps34-Beclin 1 autophagic complex, of increasing class III PI3K/Vps34 kinase activity, and/or of increasing autophagic activity. Further polypeptides of the present invention include polypeptides which have at least 90% similarity, more preferably at least 95% similarity, and still more preferably at least 96%, 97%, 98% or 99% similarity to those described above.

“% similarity” for two polypeptides refers to a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489, 1981) to find the best segment of similarity between two sequences.

It is well understood by the skilled artisan that, inherent in the definition of a “ATG14L polypeptide equivalent,” is the concept that there is a limit to the number of changes that may be made within a defined portion of the molecule and still result in a molecule with an acceptable level of equivalent biological activity, e.g., ability of ATG14L to bind to Beclin 1, to bind to a class III PI3K/Vps34-Beclin 1 autophagic complex, to increase class III PI3K/Vps34 kinase activity, and/or to increase autophagic activity. “ATG14L polypeptide equivalent” is thus defined herein as any ATG14L polypeptide in which some, or most, of the amino acids may be substituted so long as the polypeptide retains substantially similar biological activity in the context of the uses set forth herein.

The term “functional fragment thereof” means a truncated sequence of the original sequence referred to that mediates some biological activity. The truncated sequence (nucleic acid or protein sequence) can vary widely in length; the minimum size being a sequence of sufficient size to provide a sequence with at least a comparable function and/or activity of the original sequence referred to, while the maximum size is not critical. In some applications, the maximum size usually is not substantially greater than that required to provide the desired activity and/or function(s) of the original sequence.

An amino acid sequence of any length is contemplated within the definition of “ATG14L polypeptide equivalent” and “ATG14L functional fragment thereof”, so long as the polypeptide retains an acceptable level equivalent biological activity, wherein the acceptable level equivalent biological activity means at least about 30%, at least about 40%, at least about 50%, at least about 60%, at least about of 70%, at least about 80%, or at least about 90%, of the biological activity of the native ATG14L molecule. For example, an ATG14L polypeptide equivalent or an ATG14L functional fragment, that is anticipated to have an acceptable level of equivalent biological activity of ATG14L, may include a polypeptide comprising at least one amino acid sequence selected from the group consisting of amino acids 75 to 95 of SEQ ID NO: 1 and amino acids 148 to 178 of SEQ ID NO: 1, a polypeptide comprising a sequence of amino acids 99 to 492 of SEQ ID NO: 1, or a polypeptide comprising at least one amino acid sequence selected from the group consisting of amino acids 75 to 95 of SEQ ID NO: 3 and amino acids 148 to 178 of SEQ ID NO: 3. The ATG14L polypeptide equivalent or ATG14L functional fragment may include all or part of these amino acid sequences. Furthermore, ATG14L polypeptide equivalents include polypeptides containing these amino acid sequences that have additional amino acids at either the C-terminal or N-terminal end. For example, the ATG14L polypeptide equivalent may include a total of greater than 1000, 500-1000, 400-499, 300-399, 200-299, 100-199, 80-99, 60-79, 50-59, 40-49, 30-39, 20-29, 10-19, 9, 8, 7, 6, 5, or 4 amino acid residues, as long as the polypeptide remains an acceptable level of equivalent biological activity of ATG14L.

A plurality of distinct proteins/polypeptides/peptides with different substitutions may easily be made and used in accordance with the invention. Additionally, in the context of the invention, an ATG14L polypeptide equivalent can be an ATG14L homologue polypeptide from any species or organism. One of ordinary skill in the art will understand that many ATG14L polypeptide equivalents would likely exist and can be identified using commonly available techniques. In addition, any ATG14L homologue polypeptide may be substituted in some, even most, amino acids and still be an “ATG14L polypeptide equivalent,” so long as the polypeptide retains substantially similar activity in the context of the uses set forth herein.

The term “biological activity” with regard to an ATG14L or Rubicon (as described below) polypeptide, as used herein, refers to physical/chemical properties and biological functions associated with ATG14L or Rubicon. In some embodiments, the “biological activity” of ATG14L includes one or more of: binding to a class III PI3K/Vps34-Beclin 1 autophagic complex, binding to Beclin 1, increasing the class III PI3K/Vps34 kinase activity, and increasing the autophagic activity. In some embodiments, the “biological activity” of Rubicon includes one or more of: binding to a class III PI3K/Vps34-Beclin 1 autophagic complex, decreasing the class III PI3K/Vps34 kinase activity, and decreasing the autophagic activity.

As used herein, “inhibit”, “down-regulate”, or “decrease”, means that the expression of the gene, or level of RNA molecules or equivalent RNA molecules encoding one or more proteins or protein subunits, or level of protein, or biological activity of one or more proteins or protein subunits, is reduced below the normal level. For example, but not by way of limitation, decreasing or inhibiting gene expression may reduce the level of expression to a level that is too low for the gene product to perform its normal cellular function.

The term “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

The “tag” for detecting the fusion protein as used herein, includes, but are by no means limited to, chloramphenicol transferase (CAT), β-galactosidase (β-gal), luciferase, green fluorescent protein (GFP), yellow fluorescent protein (YFP), alkaline phosphatase, and other genes that can be detected, e.g., immunologically (by antibody assay).

### Rubicon

Rubicon was previously referred to in U.S. Provisional Application No. 61/096,724 as “BIRC.” The present invention pertains to various Rubicon polypeptides, nucleic acids and antibodies and their uses.

For example, various embodiments of the present invention pertain to methods for decreasing autophagic activity or decreasing class III PI3K/Vps34 kinase activity which comprise administering to a cell an effective amount of an agent which increases or enhances the biological activity of Rubicon. An agent that increases or enhances the activity of Rubicon includes, but is not limited to, Rubicon itself, a functional agonistic fragment thereof, a Rubicon mimetic compound, a therapeutic vector which comprises a nucleic acid molecule encoding Rubicon protein, and a binding enhancer which enhances or prolongs the binding between Rubicon and a class III PI3K/Vps34-Beclin 1 autophagic complex.

Other non-limiting embodiments pertain to methods of increasing autophagic activity or increasing class III PI3K/Vps34 kinase activity in a cell which comprise administering to the cell an effective amount of an agent which decreases or inhibits the biological activity of Rubicon. An agent that decreases or inhibits the biological activity of Rubicon includes, but is not limited to, a functional antagonistic fragment of Rubicon, an anti-Rubicon antibody or fragment thereof such as an intrabody, another agent which inhibits or blocks Rubicon biological activity, or a nucleic acid targeted to the Rubicon gene, such as an antisense nucleic acid, a DNA construct for expression of an antisense RNA, a ribozyme, a DNA construct for expression of a ribozyme, a DNAzyme; or an RNAi.

The full-length amino acid sequence of murine Rubicon (GenBank accession number: AAH67390; gi|45708948) has 941 amino acids, which is provided below, and is designated SEQ ID NO: 2.

(SEQ ID NO: 2)
MRPEGAGMDLGGGDGERLLEKSRREHWQLLGNLKTTVEGLVSANCPNVWSK
YGGLERLCRDMQNILYHGLIHDQVCCRQADYWQFVKDIRWLSPHSALHVEKFISLHESD
QSDTDSVSERAVAELWLQHSLQCHCLSAQLRPLLGDRQYIRKFYTETAFLLSDAHVTA
MLQCLEAVEQNNPRLLAQIDASMFARKQESPLLVTKSQSLTALPGSTYTPPASYAQHSY
FGSSSSLQSMPQSSHSSERRSTSFSLSGPSWQPQEDRECLSPAETQTTPAPLPSDSTLAQDS
PLTAQEMSDSTLTSPLEASWVSSQNDSPSDVSEGPEYLAIGNPAPHGRTASCESHSSNGE
SSSSHLFSSSSSQKLESAASSLGDQEEGRQSQAGSVLRRSSFSEGQTAPVASGTKKSHIRS
HSDTNIASRGAAEGGQYLCSGEGMFRRPSEGQSLISYLSEQDFGSCADLEKENAHFSISES
LIAAIELMKCNMMSQCLEEEEVEEEDSDREIQELKQKIRLRRQQIRTKNLLPAYRETENG
SFRVTSSSSQFSSRDSTQLSESGSAEDADDLEIQDADIRRSAVSNGKSSFSQNLSHCFLHST
SAEAVAMGLLKQFEGMQLPAASELEWLVPEHDAPQKLLPIPDSLPISPDDGQHADIYKL
RIRVRGNLEWAPPRPQIIFNVHPAPTRKIAVAKQNYRCAGCGIRTDPDYIKRLRYCEYLG
KYFCQCCHENAQMVVPSRILRKWDFSKYYVSNFSKDLLLKIWNDPLFNVQDINSALYR
KVKLLNQVRLLRVQLYHMKNMFKTCRLAKELLDSFDVVPGHLTEDLHLYSLSDLTATK
KGELGPRLAELTRAGAAHVERCMLCQAKGFICEFCQNEEDVIFPFELHKCRTCEECKAC
YHKTCFKSGRCPRCERLQARRELLAKQSLESYLSDYEEEPTEALALEATVLETT

The full-length amino acid sequence of human Rubicon has 972 amino acids, which is provided below, and is designated SEQ ID NO: 4.

(SEQ ID NO: 4)
MRPEGAGMELGGGEERLPEESRREHWQLLGNLKTTVEGLVSTNSPNVWSKYG
GLERLCRDMQSILYHGLIRDQACRRQTDYWQFVKDIRWLSPHSALHVEKFISVHENDQS
SADGASERAVAELWLQHSLQYHCLSAQLRPLLGDRQYIRKFYTDAAFLLSDAHVTAML
QCLEAVEQNNPRLLAQIDASMFARKHESPLLVTKSQSLTALPSSTYTPPNSYAQHSYFGS
FSSLHQSVPNNGSERRSTSFPLSGPPRKPQESRGHVSPAEDQTIQAPPVSVSALARDSPLT
PNEMSSSTLTSPIEASWVSSQNDSPGDASEGPEYLAIGNLDPRGRTASCQSHSSNAESSSS
NLFSSSSSQKPDSAASSLGDQEGGGESQLSSVLRRSSFSEGQTLTVTSGAKKSHIRSHSDT
SIASRGAPESCNDKAKLRGPLPYSGQSSEVSTPSSLYMEYEGGRYLCSGEGMFRRPSEGQ
SLISYLSEQDFGSCADLEKENAHFSISESLIAAIELMKCNMMSQCLEEEEVEEEDSDREIQ
ELKQKIRLRRQQIRTKNLLPMYQEAEHGSFRVTSSSSQFSSRDSAQLSDSGSADEVDEFEI
QDADIRRNTASSSKSFVSSQSFSHCFLHSTSAEAVAMGLLKQFEGMQLPAASELEWLVP
EHDAPQKLLPIPDSLPISPDDGQHADIYKLRIRVRGNLEWAPPRPQIIFNVHPAPTRKIAVA
KQNYRCAGCGIRTDPDYIKRLRYCEYLGKYFCQCCHENAQMAIPSRVLRKWDFSKYYV
SNFSKDLLIKIWNDPLFNVQDINSALYRKVKLLNQVRLLRVQLCHMKNMFKTCRLAKE
LLDSFDTVPGHLTEDLHLYSLNDLTATRKGELGPRLAELTRAGATHVERCMLCQAKGFI
CEFCQNEDDIIFPFELHKCRTCEECKACYHKACFKSGSCPRCERLQARREALARQSLESY
LSDYEEEPAEALALEAAVLEAT

Rubicon protein is predicted to comprise a conserved RUN domain, near the N-terminus, a cysteine-rich domain at the C-terminus, and a coiled-coil domain (CCD) or motif in the central region. The central region which comprises the CCD of Rubicon is shown to be important for the efficient binding/interaction between Rubicon and class III PI3K/Vps34-Beclin 1 autophagic complex. The predicted CCD of murine Rubicon has a sequence of amino acid sequences 488 to 508 of SEQ ID NO: 2. The predicted CCD of human Rubicon has a sequence of amino acid sequences 518 to 538 of SEQ ID NO: 4.

In addition, the C-terminal region of Rubicon (amino acids 837-890 of SEQ ID NO: 2 or amino acids 868-921 of SEQ ID NO: 4) functions in promotion of abnormal late-endosomal/lysosomal structure, and is likely directly involved in inhibition of autophagosome maturation. Thus, this C-terminal region can be a target for altering autophagic degradation in cells.

A native murine Rubicon polypeptide is a consecutive amino acid segment of SEQ ID NO: 2, including the full length sequence of SEQ ID NO: 2. In non-limiting embodiments, a Rubicon polypeptide comprises all or a portion (e.g., up to 100%, or up to 99% or up to 95%) of SEQ ID NO:2, and comprises a region that binds to Beclin 1. In one non-limiting embodiment, the murine Rubicon polypeptide comprises a sequence of amino acids 208 to 836 of SEQ ID NO: 2. In one non-limiting embodiment, the murine Rubicon polypeptide comprises a sequence of amino acids 1 to 836 of SEQ ID NO: 2. In another non-limiting embodiment, the murine Rubicon polypeptide comprises a sequence of amino acids 208 to 941 of SEQ ID NO: 2.

A native human Rubicon polypeptide is a consecutive amino acid segment of SEQ ID NO: 4, including the full length sequence of SEQ ID NO: 4. In non-limiting embodiments, a Rubicon polypeptide comprises all or a portion (e.g., up to 100%, or up to 99%, or up to 95%) of SEQ ID NO:4, and comprises a region that binds to Beclin 1.

One of ordinary skill in the art would understand how to generate a Rubicon polypeptide in view of the disclosure of SEQ ID NO: 2 and SEQ ID NO: 4 using any of a number of experimental methods well-known to those of skill in the art. In one embodiment, a Rubicon polypeptide having biological activity of a native Rubicon protein, the biological activity of a native Rubicon protein is as described in the examples, including, but not limited to, ability of Rubicon to bind to a class III PI3K/Vps34-Beclin 1 autophagic complex, to decrease class III PI3K/Vps34 kinase activity, and/or to decrease autophagic activity.

The polypeptides of the present invention are preferably provided in an isolated form, and preferably are substantially purified. A recombinantly produced version of the Rubicon polypeptide can be substantially purified by the one-step method described at Smith and Johnson, Gene (1988); 67:31-40. Polypeptides of the invention also can be purified from natural or recombinant sources using anti-Rubicon antibodies of the invention in methods which are well known in the art of protein purification.

In one non-limiting embodiment, the present invention provides an isolated polypeptide that has an amino acid sequence up to 99% or up to 95% identical to SEQ ID NO: 2 and comprises at least a sequence of amino acids 488 to 508 of SEQ ID NO: 2. In one non-limiting embodiment, the polypeptide comprises a sequence of amino acids 208 to 836 of SEQ ID NO: 2. In one non-limiting embodiment, the polypeptide comprises a sequence of amino acids 1 to 836 of SEQ ID NO: 2. In another non-limiting embodiment, the polypeptide comprises a sequence of amino acids 208 to 941 of SEQ ID NO: 2. In another non-limiting embodiment, the present invention provides an isolated polypeptide that has an amino acid sequence up to 99% or up to 95% identical to SEQ ID NO: 4 and comprises at least a sequence of amino acids 518 to 538 of SEQ ID NO: 4. In non-limiting embodiments, a polypeptide which is “up to 95% identical” or “up to 99” identical” to SEQ ID NO:2 is also greater than 50%, greater than 55%, greater than 60%, greater than 65%, greater than 70%, greater than 75%, greater than 80%, greater than 85%, or greater than 90% identical to SEQ ID NO:2, and a polypeptide which is “up to 95% identical” or “up to 99” identical” to SEQ ID NO:4 is also greater than 50%, greater than 55%, greater than 60%, greater than 65%, greater than 70%, greater than 75%, greater than 80%, greater than 85%, or greater than 90% identical to SEQ ID NO:4. The polypeptides of the present invention also include polypeptides having an amino acid sequence at least 80% identical, more preferably at least 90% identical, and still more preferably 95%, 96%, 97%, 98% or 99% identical to those described above, as well as polypeptides having an amino acid sequence with at least 90% similarity, and more preferably at least 95% similarity, to those described above. Preferably, the aforementioned polypeptides are capable of binding to a class III PI3K/Vps34-Beclin 1 autophagic complex, of decreasing class III PI3K/Vps34 kinase activity, and/or of decreasing autophagic activity. Further polypeptides of the present invention include polypeptides which have at least 90% similarity, more preferably at least 95% similarity, and still more preferably at least 96%, 97%, 98% or 99% similarity to those described above.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489, 1981) to find the best segment of similarity between two sequences.

It is well understood by the skilled artisan that, inherent in the definition of a “Rubicon polypeptide equivalent,” is the concept that there is a limit to the number of changes that may be made within a defined portion of the molecule and still result in a molecule with an acceptable level of equivalent biological activity, e.g., ability of Rubicon to bind to a class III PI3K/Vps34-Beclin 1 autophagic complex, to decrease class III PI3K/Vps34 kinase activity, and/or to decrease autophagic activity. “Rubicon polypeptide equivalent” is thus defined herein as any Rubicon polypeptide in which some, or most, of the amino acids may be substituted so long as the polypeptide retains substantially similar biological activity in the context of the uses set forth herein.

The term “functional fragments thereof” means a truncated sequence of the original sequence referred to that mediates biological activity. The truncated sequence (nucleic acid or protein sequence) can vary widely in length; the minimum size being a sequence of sufficient size to provide a sequence with at least a comparable function and/or biological activity of the original sequence referred to, while the maximum size is not critical. In some applications, the maximum size usually is not substantially greater than that required to provide the desired activity and/or function(s) of the original sequence.

An amino acid sequence of any length is contemplated within the definition of “Rubicon polypeptide equivalent” and “Rubicon functional fragments thereof”, so long as the polypeptide retains an acceptable level of equivalent biological activity, wherein the acceptable level equivalent biological activity means at least about 30%, at least about 40%, at least about 50%, at least about 60%, at least about of 70%, at least about 80%, or at least about 90%, of the biological activity of the native Rubicon molecule For example, a Rubicon polypeptide equivalent or a Rubicon functional fragment thereof, that is anticipated to have an acceptable level of equivalent biological activity of Rubicon, may include a polypeptide comprising at least a sequence of amino acids 488-508 of SEQ ID NO: 2, a polypeptide comprising a sequence of amino acids 208 to 836 of SEQ ID NO: 2, a polypeptide comprising a sequence of amino acids 1 to 836 of SEQ ID NO: 2, a polypeptide comprising a sequence of amino acids 208 to 941 of SEQ ID NO: 2, or a polypeptide comprising at least a sequence of amino acids 518-538 of SEQ ID NO: 4. These amino acid sequences are part of murine Rubicon and human Rubicon, respectively. The Rubicon polypeptide equivalent or Rubicon functional fragment may include all or part of these amino acid sequences. Further, Rubicon polypeptide equivalents include polypeptides containing these amino acid sequences that have additional amino acids at either the C-terminal or N-terminal end. For example, the Rubicon polypeptide equivalent may include a total of greater than 1000, 500-1000, 400-499, 300-399, 200-299, 100-199, 80-99, 60-79, 50-59, 40-49, 30-39, 20-29, 10-19, 9, 8, 7, 6, 5, or 4 amino acid residues, as long as it remains an acceptable level of equivalent biological activity of Rubicon.

Of course, a plurality of distinct proteins/polypeptides/peptides with different substitutions may easily be made and used in accordance with the invention. Additionally, in the context of the invention, a Rubicon polypeptide equivalent can be a Rubicon homologue polypeptide from any species or organism, including, but not limited to, a human polypeptide. One of ordinary skill in the art will understand that many Rubicon polypeptide equivalents would likely exist and can be identified using commonly available techniques. In addition, any Rubicon homologue polypeptide may be substituted in some, even most, amino acids and still be a “Rubicon polypeptide equivalent,” so long as the polypeptide retains substantially similar biological activity in the context of the uses set forth herein.

### Antibodies

The present invention provides both isolated anti-ATG14L antibodies or antigen binding portions thereof, and anti-Rubicon antibodies or antigen binding portions thereof.

The term “antibody” as used herein refers to intact immunoglobulins as well as antigen binding fragments (i.e., “antigen-binding portion”) or single chains thereof which bind to ATG14L or Rubicon, although in certain instances, an “antibody fragment” or “antigen binding portion” is expressly recited. An “intact immunoglobulin” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antibody fragment” and “antigen-binding portion” of an antibody (or simply “antibody portion”), as used herein, refer to one or more fragments of an antibody that retain the ability to specifically bind to an antigen (e.g., ATG14L or Rubicon). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3^{rd} ed. 1993); (iv) a Fd fragment consisting of the V_{H} and C_{H}1 domains; (v) a Fv fragment consisting of the V_{L}, and V_{H} domains of a single arm of an antibody, (vi) a dAb fragment (Ward et al., (1989) Nature 341:544-546), which consists of a V_{E} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. Science (1988); 242:423-426; and Huston et al. Proc. Natl. Acad. Sci. USA (1988); 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds ATG14L or Rubicon is substantially free of antibodies that specifically bind antigens other than ATG14L or Rubicon). An isolated antibody that specifically binds ATG14L or Rubicon may, however, have cross-reactivity to other antigens, such as ATG14L or Rubicon molecules from other species.

The term “intrabodies” as used herein, refers to antibodies, often scFvs, that are expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies of the invention can be produced using methods known in the art, such as those disclosed and reviewed in Chen et al., Hum. Gene Ther. (1994); 5:595-601; Marasco, W. A., Gene Ther. (1997); 4:11-15; Rondon and Marasco, Annu. Rev. Microbial. (1997); 51:257-283; Proba et al., J. Mol. Biol. (1998); 275:245-253; Cohen et al., Oncogene (1998); 17:2445-2456; Ohage and Steipe, J. Mol. Biol. (1999); 291:1119-1128; Ohage et al., J. Mol. Biol. (1999); 291:1129-1134; Wirtz and Steipe, Protein Sci. (1999); 8:2245-2250; Zhu et al., J. Immunol. Methods 231:207-222 (1999); and references cited therein.

### Detection of Expression

The present invention provides a method of evaluating expression of ATG14L or Rubicon in a cell in a biological sample. In one embodiment, the method of evaluating expression comprises detecting an ATG14L or Rubicon polypeptide in the biological sample. In another embodiment, the method of evaluating expression comprises detecting the amount of ATG14L or Rubicon mRNA in the biological sample.

In one non-limiting embodiment, the expression of ATG14L correlates to autophagic activity. For example, increased expression of ATG14L indicates increased autophagic activity, and decreased expression of ATG14L indicates decreased autophagic activity. In another non-limiting embodiment, the expression of ATG14L correlates to class III PI3K/Vps34 kinase activity. For example, increased expression of ATG14L indicates increased class III PI3K/Vps34 kinase activity, and decreased expression of ATG14L indicates decreased class III PI3K/Vps34 kinase activity.

In one non-limiting embodiment, the expression of Rubicon correlates to autophagic activity. For example, increased expression of Rubicon indicates decreased autophagic activity, and decreased expression of Rubicon indicates increased autophagic activity. In another non-limiting embodiment, the expression of Rubicon correlates to class III PI3K/Vps34 kinase activity. For example, increased expression of Rubicon indicates decreased class III PI3K/Vps34 kinase activity, and decreased expression of Rubicon indicates increased class III PI3K/Vps34 kinase activity.

In one non-limiting embodiment, the method of evaluating expression of ATG14L or Rubicon comprises detecting an ATG14L or Rubicon polypeptide in the biological sample, which method comprises (a) contacting the biological sample with an anti-ATG14L or anti-Rubicon antibody or antigen binding portion thereof and (b) detecting the presence of an anti-ATG14L or anti-Rubicon antibody or the antigen binding portion thereof that is specifically bound to ATG14L or Rubicon polypeptide from the biological sample. The methods include, but are not limited to, Enzyme-Linked ImmunoSorbent Assay (ELISA), a Western blot, labeling the ATG14L polypeptide and identifying the labeled ATG14L polypeptide, a mass spectrometry, a gel electrophoresis, and a combination thereof.

In one non-limiting embodiment, the method of evaluating expression comprises detecting the amount of ATG14L or Rubicon mRNA in the biological sample. The methods include, but are not limited to a reverse transcription-polymerase chain reaction, Northern blotting, microarray, or a combination thereof.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decreases production. Molecules (e.g., transcription factors) that are involved in up-regulation or down-regulation are often called “activators” and “repressors,” respectively.

### Detecting Polypeptide

A preferred agent for detecting ATG14L or Rubicon polypeptide is an antibody capable of binding to ATG14L or Rubicon polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The detection method of the invention can be used to detect ATG14L or Rubicon activity in a biological sample in vitro as well as in vivo. In vitro techniques for detection of ATG14L or Rubicon polypeptide include, but are not limited to, enzyme linked immunosorbent assay (ELISA), Western blot, labeling the ATG14L or Rubicon polypeptide and identifying the labeled ATG14L or Rubicon polypeptide using a technique such as immunofluorescence, mass spectrometry, gel electrophoresis, or immunoprecipitation. For a detailed explanation of methods for carrying out Western blot analysis (Sambrook et al., Molecular Cloning, A Laboratory Manual (2nd ed. 1989) at Chapter 18). The protein detection and isolation methods employed herein may also be such as those described in for example, Harlow, E. and Lane, D., 1988, “Antibodies: A Laboratory Manual,” Cold Spring Harbor Laboratory Press, Cold Spring Harbor, N.Y.

The term “biological sample” is intended to mean any biological sample obtained from an individual subject, including but not limited to a body fluid or a tissue sample, cell line, tissue culture, etc. Examples of body fluids include blood, semen, serum, plasma, urine, synovial fluid and spinal fluid.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

Detection of ATG14L or Rubicon activity can be accomplished, for example, by immunofluorescence techniques employing a fluorescently labeled antibody (see below) coupled with light microscopic, flow cytometric, or fluorimetric detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-ATG14L or anti-Rubicon polypeptide specific antibody is via linkage to an enzyme and use in an enzyme immunoassay (EIA) (Voller, “The Enzyme Linked Immunosorbent Assay (ELISA),” Diagnostic Horizons 2:1-7, 1978, Microbiological Associates Quarterly Publication, Walkersville, Md.; Voller, et al., J. Clin. Pathol. (1978); 31:507-520; Butler, Meth. Enzymol. (1981); 73:482-523; Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

ELISA comprises preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISA, the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISA see, e.g., Ausubel et al, eds, 1994, Current Protocols in Molecular Biology, Vol. 1, John Wiley & Sons, Inc., New York at 11.2.1.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as .sup.152Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Furthermore, in vivo techniques for detection of ATG14L or Rubicon protein include introducing into a subject a labeled anti-ATG14L or anti-Rubicon antibody. For example, the antibody can be labeled with a radioactive marker whose presence and location in a subject can be detected by standard imaging techniques.

In addition, the polypeptides in the sample are fractionated based on a physio-chemical characteristic of the polypeptide. A most useful method of separation is molecular weight, as there are many useful methods to separate proteins based on this characteristic including, for example, SDS gel electrophoresis and gas phase ion spectrometry, e.g., mass spectrometry. Another useful physiochemical characteristic is isoelectric point. Isoelectric focusing, affinity chromatography and solid phase extraction on an ion exchange resin will fractionate proteins in a sample based on this property.

Methods of fractionating proteins are used to examine the level of expression of a selected protein in a cell. The techniques described herein can be used to examine one or more proteins expressed in a cell, up to tens, hundreds, thousands, or tens of thousands of proteins. Any one technique or a combination of techniques can be used to fractionate the proteins, based on one or more physio-chemical property. Methods of fractionation include, e.g., two dimensional gels; capillary gel electrophoresis; mass spectrometry, e.g., MALDI, SELDI; ICAT (isotope coded affinity tag, see, e.g., Mann, Nature Biotechnology (1999); 17:954-955; Gygi et al., Nature Biotechnology (1999); 17:994-999); chromatography, e.g., gel-filtration, ion-exchange, affinity, immunoaffinity, and metal chelate chromatography, HPLC, e.g., reversed phase, ion-exchange, and size exclusion HPLC; western blotting; immunohistochemistry techniques such as ELISA and in situ screening with antibodies, etc (see, e.g., Blackstock & Weir, Trends in Biotech. (1999); 17:121-127=; Dutt & Lee, Biochemical Engineering, pages 176-179 (April 2000); Page et al., Drug Discovery Today (1999); 4:55-62; Wang and Newick, Drug Discovery Today (1999); 4:129-133; Regnier et al., Trends in Biotech. (1999); 17:101-106; and Pandey and Mann, Nature (2000); 405:837-846). The proteins of interest are identified and isolated using techniques known to those of skill in the art.

For a general description of these techniques, see also Sambrook et al., Molecular Cloning, A Laboratory Manual (2nd ed. 1989); and Current Protocols in Molecular Biology (Ausubel et al., eds., 1994).

Two-dimensional electrophoresis can be used to fractionate the proteins of the invention. This technique fractionates proteins based on the physio-chemical characteristics of pI and molecular weight. 2d gel electrophoresis and the techniques described herein can be used alone, or in combination with other techniques such as mass spectrometry, e.g., MALDI and SELDI. MALDI is a mass spectrometry technique that fractionates proteins based on mass, and is often combined with size and or affinity chromatography techniques to increase resolution.

Detecting mRNA

Methods for evaluating gene expression by detecting the amount of mRNA level in a cell, often but not always hybridization based, include, e.g., northern blots; dot blots; primer extension; nuclease protection; subtractive hybridization and isolation of non-duplexed molecules using, e.g., hydroxyapatite; solution hybridization; filter hybridization; amplification techniques such as RT-PCR and other PCR-related techniques such as differential display, LCR, AFLP, RAP, etc. (see, e.g., U.S. Pat. Nos. 4,683,195 and 4,683,202; PCR Protocols: A Guide to Methods and Applications (Innis et al., eds, 1990); Liang and Pardee, Science 257:967-971 (1992); Hubank & Schatz, Nuc. Acids Res. 22:5640-5648 (1994); Perucho et al., Methods Enzymol. 254:275-290 (1995)), fingerprinting, e.g., with restriction endonucleases (Ivanova et al., Nuc. Acids. Res. 23:2954-2958 (1995); Kato, Nuc. Acids Res. 23:3685-3690 (1995); and Shimkets et al., Nature Biotechnology 17:798-803, see also U.S. Pat. No. 5,871,697)); and the use of structure specific endonucleases (see, e.g., De Francesco, The Scientist 12:16 (1998)).

Nucleotide probes can be used to detect expression of a gene corresponding to the provided polynucleotide. In Northern blots, mRNA is separated electrophoretically and contacted with a probe. A probe is detected as hybridizing to an mRNA species of a particular size. The amount of hybridization can be quantified to determine relative amounts of expression. Probes can be used for in situ hybridization to cells to detect expression. Probes can also be used in vivo for diagnostic detection of hybridizing sequences. Probes can be labeled with a radioactive isotope or other types of detectable labels, e.g., chromophores, fluorophores and/or enzymes. Other examples of nucleotide hybridization assays are described in WO92/02526 and U.S. Pat. No. 5,124,246.

PCR is another means for detecting small amounts of target nucleic acids (see, e.g., Mullis et al., Meth. Enzymol. (1987) 155:335; U.S. Pat. No. 4,683,195; and U.S. Pat. No. 4,683,202). Two primer oligonucleotides that hybridize with the target nucleic acids can be used to prime the reaction. The primers can be composed of sequence within or 3′ and 5′ to the polynucleotides described herein. After amplification of the target by standard PCR methods, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase enzyme can be purified and separated using gel electrophoresis, and transferred to a solid support, such as nitrocellulose. The solid support can be exposed to a labeled probe and washed to remove any unhybridized probe. Duplexes containing the labeled probe can then be detected.

The terms “reverse transcription polymerase chain reaction” and “RT-PCR” refer to a method for reverse transcription of an RNA sequence to generate a mixture of cDNA sequences, followed by increasing the concentration of a desired segment of the transcribed cDNA sequences in the mixture without cloning or purification. Typically, RNA is reverse transcribed using a single primer (e.g., an oligo-dT primer) prior to PCR amplification of the desired segment of the transcribed DNA using two primers.

For a general description of these techniques, see also Sambrook et al., Molecular Cloning, A Laboratory Manual (2nd ed. 1989), see, e.g., pages 7.37-7.39, 7.53-7.54, 7.58-7.66, and 7.71-7.79; Kriegler, Gene Transfer and Expression: A Laboratory Manual (1990); and Current Protocols in Molecular Biology (Ausubel et al., eds., 1994).

Techniques have been developed that expedite expression analysis and sequencing of large numbers of nucleic acids samples. For example, nucleic acid arrays have been developed for high density and high throughput expression analysis (see, e.g., Granjeuad et al., BioEssays 21:781-790 (1999); Lockhart & Winzeler, Nature 405:827-836 (2000)). Nucleic acid arrays refer to large numbers (e.g., hundreds, thousands, tens of thousands, or more) of nucleic acid probes bound to solid substrates, such as nylon, glass, or silicon wafers (see, e.g., Fodor et al., Science 251:767-773 (1991); Brown & Botstein, Nature Genet. 21:33-37 (1999); Eberwine, Biotechniques 20:584-591 (1996)). A single array can contain, e.g., probes corresponding to an entire genome, or to all genes expressed by the genome. The probes on the array can be DNA oligonucleotide arrays (e.g., GeneChip™, see, e.g., Lipshutz et al., Nat. Genet. 21:20-24 (1999)), mRNA arrays, cDNA arrays, EST arrays, or optically encoded arrays on fiber optic bundles (e.g., BeadArray™). The samples applied to the arrays for expression analysis can be, e.g., PCR products, cDNA, mRNA, etc.

As used herein, the term “microarray” refers to analysis of individual recombinant clones (e.g., cosmid, YAC, BAC, plasmid or other vectors) that are placed on a two-dimensional solid support (e.g., microscope slide). Each primary clone can be identified on the support by virtue of its location (row and column) on the solid support. Arrayed libraries of clones can be screened with RNA obtained from a specimen of interest upon conjugation of a fluorochrome.

A “microarray” comprises a surface with an array, preferably ordered array, of potential binding (e.g., by hybridization) sites for a biochemical sample (target) which often has undetermined characteristics. In a preferred embodiment, a microarray refers to an assembly of distinct polynucleotide or oligonucleotide probes immobilized at defined positions on a substrate. Arrays are formed on substrates fabricated with materials such as paper, glass, plastic (e.g., polypropylene, nylon, polystyrene), polyacrylamide, nitrocellulose, silicon, optical fiber or any other suitable solid or semi-solid support, and configured in a planar (e.g., glass plates, silicon chips) or three-dimensional (e.g., pins, fibers, beads, particles, microtiter wells, capillaries) configuration. Probes forming the arrays may be attached to the substrate by any number of ways including (i) in situ synthesis (e.g., high-density oligonucleotide arrays) using photolithographic techniques (see, Fodor et al., Science (1991), 251:767-773; Pease et al., Proc. Natl. Acad. Sci. U.S.A. (1994), 91:5022-5026; Lockhart et al., Nature Biotechnology (1996), 14:1675; U.S. Pat. Nos. 5,578,832; 5,556,752; and 5,510,270); (ii) spotting/printing at medium to low-density (e.g., cDNA probes) on glass, nylon or nitrocellulose (Schena et al, Science (1995), 270:467-470, DeRisi et al, Nature Genetics (1996), 14:457-460; Shalon et al., Genome Res. (1996), 6:639-645; and Schena et al., Proc. Natl. Acad. Sci. U.S.A. (1995), 93:10539-11286); (iii) by masking (Maskos and Southern, Nuc. Acids. Res. (1992), 20:1679-1684) and (iv) by dot-blotting on a nylon or nitrocellulose hybridization membrane (see, e.g., Sambrook et al., Eds., 1989, Molecular Cloning: A Laboratory Manual, 2nd ed., Vol. 1-3, Cold Spring Harbor Laboratory (Cold Spring Harbor, N.Y.)). Probes may also be noncovalently immobilized on the substrate by hybridization to anchors, by means of magnetic beads, or in a fluid phase such as in microtiter wells or capillaries. The probe molecules are generally nucleic acids such as DNA, RNA, PNA, and cDNA but may also include proteins, polypeptides, oligosaccharides, cells, tissues and any permutations thereof which can specifically bind the target molecules.

Additional techniques for rapid gene sequencing and analysis of gene expression include, e.g., SAGE (serial analysis of gene expression). For SAGE, a short sequence tag (typically about 10-14 bp) contains sufficient information to uniquely identify a transcript. These sequence tags can be linked together to form long serial molecules that can be cloned and sequenced. Quantitation of the number of times a particular tag is observed proves the expression level of the corresponding transcript (see, e.g., Velculescu et al., Science 270:484-487 (1995); Velculescu et al., Cell 88 (1997); and de Waard et al., Gene 226:1-8 (1999)).

### Peptides and Peptide Mimetics

The term “peptide” is used in its broadest sense to refer to a compound of two or more subunit amino acids, amino acid analogs or peptidomimetics. The subunits may be linked by peptide bonds. As used herein the term “amino acid” may refer to natural, unnatural or synthetic amino acids, including the D or L optical isomers, amino acid analogs and peptidomimetics. Thus, peptides of the invention may, optionally comprise D-amino acids, a combination of D- and L-amino acids, and various “designer” amino acids (e.g., β-methyl amino acids, Cα-methyl amino acids, and Nα.-methyl amino acids, etc.) to convey special properties to peptides.

Polypeptides of the present invention may be isolated and purified natural products, or may be produced partially or wholly using recombinant chemical synthesis techniques.

“Peptide mimetics” or “peptidomimetics” are described in Fauchere, J. (1986) Adv. Drug Res. 15:29; Veber and Freidinger (1985) TINS p. 392; and Evans et al. (1987) J. Med. Chem. 30:1229. Peptide mimetics that are structurally similar to therapeutically useful peptides may be used to produce an equivalent therapeutic effect. Generally, peptidomimetics are structurally similar to a paradigm polypeptide (i.e., a polypeptide that has a biological or pharmacological activity), but have one or more peptide linkages replaced by a linkage selected from the group consisting of: —CH₂NH—, —CH₂S—, —CH₂CH₂2-, —CH═CH-(cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends Pharm Sci (1980) pp. 463-468 (general review); Hudson, D. et al., Int J Pept Prot Res (1979) 14:177-185 (—CH₂NH—, CH₂CH₂—); Spatola, A. F. et al., Life Sci (1986) 38:1243-1249 (—CH₂S); Haim, M. M., J. Chem Soc Perkin Trans I (1982) 307-314 (—CH—CH—, cis and trans); Almquist, R. G. et al., J Med Chem (1980) 23:1392-1398 (—COCH₂—); Jennings-White, C. et al., Tetrahedron Lett (1982) 23:2533 (—COCH₂—); Szelke, M. et al., European Appln. EP 45665 (1982) CA: 97:39405 (1982) (—CH(OH)CH₂—); Holladay, M. W. et al., Tetrahedron Lett (1983) 24:4401-4404 (—C(OH)CH₂—); and Hruby, V. J., Life Sci (1982) 31:189-199 (—CH₂S—).

Peptide mimetics may have significant advantages over polypeptide embodiments, including, for example: more economical production; greater chemical stability; enhanced pharmacological properties (half-life, absorption, potency, efficacy, etc.); altered specificity (e.g., a broad-spectrum of biological activities); reduced antigenicity; and others.

ATG14L or Rubicon protein variants can be generated through various techniques known in the art. For example, functional antagonistic fragments of the protein can be generated which are able to inhibit the function of the naturally occurring form of the protein, such as by competitively binding to another molecule that interacts with ATG14L or Rubicon protein. In addition, functional agonistic forms of the protein may be generated that constitutively express on or more ATG14L or Rubicon functional activities. Whether a change in the amino acid sequence of a peptide results in an ATG14L or Rubicon protein variant having one or more functional activities of a native ATG14L or Rubicon protein can be readily determined by testing the variant for a native ATG14L or Rubicon protein functional activity.

### Binding Enhancers

As used herein, a “binding enhancer” refers to a compound capable of enhancing the binding between two binding partners when added to a reaction solution. For example, binding between ATG14L with class III PI3K/Vps34-Beclin 1, binding between ATG14L and Beclin 1, or binding between Rubicon with class III PI3K/Vps34-Beclin 1. Non-limiting examples of binding enhancers include compounds such as glutaraldehyde or carbodiimide.

The concentration of a binding enhancer in a reaction solution may be appropriately set according to the type of binding enhancer. More specifically, in the case of glutaraldehyde, for example, the final concentration in a reaction solution is typically from 0.1 to 25%, and preferably from 0.2 to 18%. The binding enhancer may be added to a reaction solution containing a conjugate of binding partners before diluting the reaction solution. The reaction solution to which a binding enhancer has been added can be diluted after incubation at 37° C. for several seconds to about 20 seconds, preferably two to ten seconds, or two to five seconds. When glutaraldehyde or carbodiimide is used as a binding enhancer, the reaction solution may be diluted immediately after the addition.

### Nucleic Acids

The present invention provides for nucleic acid sequences encoding an ATG14L or Rubicon polypeptide, as defined herein, including but not limited to SEQ ID NO. 5 (GenBank Accession No: BC090995) encoding murine ATG14L, SEQ ID NO. 6 encoding human ATG14L, SEQ ID NO. 7 (GenBank Accession No: AK030368) encoding murine Rubicon, and SEQ ID NO. 8 (GenBank Accession No: NM_{—}014687) encoding human Rubicon, a nucleic acid encoding a polypeptide which has an amino acid sequence up to 99% or up to 95% identical to SEQ ID NO: 1 and comprises at least one amino acid sequences selected from the group consisting of amino acids 75 to 95 of SEQ ID NO: 1, and amino acids 148 to 178 of SEQ ID NO: 1, a nucleic acid encoding a polypeptide comprising a sequence of amino acids 99 to 492 of SEQ ID NO:1, a nucleic acid encoding a polypeptide which has an amino acid sequence up to 99% or up to 95% identical to SEQ ID NO: 3 and comprises at least one amino acid sequence selected from the group consisting of amino acids 75 to 95 of SEQ ID NO: 3, and amino acids 148 to 178 of SEQ ID NO: 3, a nucleic acid encoding a polypeptide which has an amino acid sequence up to 99% or up to 95% identical to SEQ ID NO: 2 and comprises at least a sequence of amino acids 488 to 508 of SEQ ID NO: 2, a nucleic acid encoding a polypeptide comprising a sequence of amino acids 208 to 836 of SEQ ID NO: 2, a nucleic acid encoding a polypeptide comprising a sequence of amino acids 1 to 836 of SEQ ID NO: 2, a nucleic acid encoding a polypeptide comprising a sequence of amino acids 208 to 941 of SEQ ID NO: 2, and a nucleic acid encoding a polypeptide which has an amino acid sequence up to 99% or up to 95% identical to SEQ ID NO: 4 and comprises at least a sequence of amino acids 518 to 538 of SEQ ID NO: 4.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

“Nucleic acid sequence,” “nucleotide sequence” and “polynucleotide sequence” as used herein, refer to an oligonucleotide or polynucleotide, and fragments or portions thereof, and to DNA or RNA of genomic or synthetic origin which may be single- or double-stranded, and represent the sense or antisense strand.

As used herein, the term “oligonucleotide” refers to a nucleic acid sequence of at least about 10 nucleotides and as many as about 50 or about 60 nucleotides, preferably about 15 to 30 nucleotides, and more preferably about 20 to 25 nucleotides.

As used herein, the term “nucleic acid molecule” refers to a molecule comprising nucleotides. The nucleic acid can be single, double, or multiple stranded and can comprise modified or unmodified nucleotides or non-nucleotides or various mixtures and combinations thereof. The terms “nucleic acid” and “nucleic acid molecule” are intended to include DNA molecules (e.g., cDNA or genomic DNA) and RNA molecules (e.g., mRNA) and analogs of the DNA or RNA generated using nucleotide analogs.

### Antisense

Antisense nucleic acid oligonucleotide molecules can include those that specifically hybridize (e.g., bind) under cellular conditions to cellular mRNA and/or genomic DNA encoding an ATG14L or Rubicon polynucleotide in a manner that inhibits expression of the ATG14L or Rubicon polypeptide, e.g., by inhibiting transcription and/or translation. The binding may be by conventional base pair complementarity, or, for example, in the case of binding to DNA duplexes, through specific interactions in the major groove of the double helix.

Antisense constructs can be delivered, for example, as an expression plasmid which, when transcribed in the cell, produces RNA which is complementary to at least a unique portion of the cellular mRNA which encodes an ATG14L or Rubicon polypeptide. Alternatively, the antisense construct can take the form of an oligonucleotide probe generated ex vivo which, when introduced into an ATG14L or Rubicon polypeptide expressing cell, causes inhibition of ATG14L or Rubicon polypeptide expression by hybridizing with an mRNA and/or genomic sequences coding for ATG14L or Rubicon polypeptide. Such oligonucleotide probes can be modified oligonucleotides that are resistant to endogenous nucleases, e.g., exonucleases and/or endonucleases; and are therefore stable in vivo. Exemplary nucleic acid molecules for use as antisense oligonucleotides are phosphoramidate, phosphothioate and methylphosphonate analogs of DNA (see, e.g., U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668. In one non-limiting embodiment, with respect to antisense DNA, oligodeoxyribonucleotides can be derived from the translation initiation site, e.g., between the −10 and +10 regions of a polypeptide encoding nucleotide sequence.

Antisense approaches involve the design of oligonucleotides (either DNA or RNA or combinations or variants thereof) that are complementary to ATG14L or Rubicon mRNA. The antisense oligonucleotides will bind to ATG14L or Rubicon mRNA transcripts and inhibit translation. Absolute complementarity is not required. The ability to hybridize will depend on both the degree of complementarity and the length of the antisense nucleic acid. Generally, the longer the hybridizing nucleic acid, the more base mismatches with an RNA it may contain and still form a stable duplex or triplex. One skilled in the art can ascertain a tolerable degree of mismatch by use of standard procedures to determine the melting point of the hybridized complex. Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. Wagner, R. Nature 372:333 (1994). Therefore, oligonucleotides complementary to either the 5′ or 3′ untranslated, non-coding regions of an ATG14L or Rubicon gene could be used in an antisense approach to inhibit translation of endogenous ATG14L or Rubicon mRNA. Oligonucleotides complementary to the 5′ untranslated region of the mRNA can include the complement of the AUG start codon. Although antisense oligonucleotides complementary to mRNA coding regions are generally less efficient inhibitors of translation, these could still be used. Whether designed to hybridize to the 5′, 3′ or coding region of ATG14L or Rubicon mRNA, antisense nucleic acids can be less that about 100 (e.g., less than about 30, 29, 28, 27, 26, 25, 24, 23, 22, 21, 20, 19 or 18) nucleotides in length. Generally, in order to be effective, the antisense oligonucleotide could be 18 or more nucleotides in length, but may be shorter depending on the conditions. Accordingly, antisense nucleic acids may preferably be, in non-limiting embodiments, at least about 10 or at least about 12 or at least about 15 or at least about 18 nucleotides and less than about 30 or less than about 28 or less than about 25, or less than about 23, or less than about 20 nucleotides in length.

Specific antisense oligonucleotides can be tested for effectiveness using in vitro studies to assess the ability of the antisense oligonucleotide to inhibit gene expression. Such studies can (1) utilize controls (e.g., a non-antisense oligonucleotide of the same size as the antisense oligonucleotide) to distinguish between antisense gene inhibition and nonspecific biological effects of oligonucleotides, and (2) compare levels of the target RNA or protein with that of an internal control RNA or protein.

Oligonucleotides can be synthesized by standard methods known in the art, e.g., by use of an automated DNA synthesizer, as described in Ausubel and Sambrook. Phosphorothioate oligonucleotides may be synthesized by the method of Stein et al. (1988) Nucl. Acids Res. 16:3209. Methylphosphonate oligonucleotides can be prepared by use of controlled pore glass polymer supports (e.g., as described in Sarin et al. (1988) Proc. Natl. Acad. Sci. U.S.A. 85:7448-7451).

A number of methods have been developed for delivering antisense DNA or RNA into cells. For example, antisense molecules can be introduced directly into a cell by electroporation, liposome-mediated transfection, CaCl-mediated transfection, or using a gene gun. Modified nucleic acid molecules designed to target the desired cells (e.g., antisense oligonucleotides linked to peptides or Abs that specifically bind receptors or antigens expressed on the target cell surface) can be used. To achieve high intracellular concentrations of antisense oligonucleotides (as may be required to suppress translation on endogenous mRNAs), an exemplary approach utilizes a recombinant DNA construct in which the antisense oligonucleotide is placed under the control of a strong promoter (e.g., the CMV promoter).

Ribozymes

Ribozyme oligonucleotide molecules designed to catalytically cleave ATG14L or Rubicon mRNA transcripts can also be used to prevent translation of ATG14L or Rubicon mRNAs and expression of ATG14L or Rubicon proteins. As one non-limiting example, hammerhead ribozymes that cleave mRNAs at locations dictated by flanking regions that form complementary base pairs with the target mRNA might be used so long as the target mRNA has the following common sequence: 5′-UG-3′. As another example, hairpin and hepatitis delta virus ribozymes may also be used. To increase efficiency and minimize the intracellular accumulation of non-functional mRNA transcripts, a ribozyme should be engineered so that the cleavage recognition site is located near the 5′ end of the target ATG14L or Rubicon mRNA. Ribozymes can be delivered to a cell using a vector as described below.

Antisense RNA, DNA, or ribozyme can be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis. RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducibly, depending on the promoter used, can be introduced stably into cell lines.

RNA Interference (RNAi)

As used herein, the term “RNA interference,” or “RNAi,” refers to the process whereby sequence-specific, post-transcriptional gene silencing is initiated by an RNA oligonucleotide that is homologous in sequence to the silenced gene. RNAi, which occurs in a wide variety of living organisms, has also been referred to as post-transcriptional gene silencing (PTGS) and co-suppression. The sequence-specific degradation of mRNA observed in RNAi, is mediated by small (or short) interfering RNAs (siRNAs).

As used herein, the term “RNAi oligonucleotide,” “RNAi” or “RNAi agent” means an RNA molecule capable of directing the degradation of an RNA transcript having a nucleotide sequence at least a portion of which is substantially the same as that of the interfering RNA, through the mechanism of RNA interference (RNAi). For example, “RNAi oligonucleotide,” “RNAi” or “RNAi agent” can down-regulate the expression of a target gene, e.g., an ATG14L gene, and preferably a human ATG14L gene or a murine ATG14L gene, and a Rubicon gene, and preferably a human Rubicon gene or a murine Rubicon gene. As known in the art, RNAi oligonucleotides can be “small interfering RNAs,” or siRNAs, composed of two complementary single-stranded RNAs that form an intermolecular duplex. RNAi oligonucleotides can also be “short hairpin RNAs,” or shRNAs, composed of a single-stranded RNA with two self-complementary regions that allow the RNA to fold back upon itself and form a stem-loop structure with an intramolecular duplex region and an unpaired loop region. Finally, in some circumstances (See Martinez et al., Cell (2002); 110:563-574), interfering RNAs can be single-stranded antisense RNAs of 19 to 29 nucleotides that are complementary to a target sequence.

The terms “small interfering RNA” (also sometimes referred to as short interfering RNA) or “siRNA,” as used herein, refer to the mediators of RNAi-RNA molecules capable of directing sequence-specific, post-transcriptional gene silencing of specific genes with which they share nucleotide sequence identity or similarity. Recent experiments have shown that the siRNAs that are most effective in mammalian cells are duplexes composed or two complementary 21 nucleotide single-stranded RNAs that anneal to form a duplexed region of 19 basepairs and single-stranded overhangs of 2 nucleotides at their 3′ ends. In some organisms (e.g., C. elegans, D. melanogaster and various plants) these siRNAs can be created by the nucleolytic processing of longer dsRNAs. In mammalian cells they apparently can also be produced from short (i.e., less than 30 basepairs) hairpin RNAs, or shRNAs. Accordingly, siRNAs may preferably be, in non-limiting embodiments, at least about 10 or at least about 12 or at least about 15 or at least about 18 nucleotides and less than about 30 or less than about 28 or less than about 25, or less than about 23, or less than about 20 nucleotides in length. In one non-limiting embodiment, a siRNA has a nucleotide sequence of SEQ ID NO: 11. In one non-limiting embodiment, a siRNA has a nucleotide sequence of SEQ ID NO: 12.

The term “small hairpin siRNA,” “short hairpin siRNA,” or “shRNAs,” as used herein, refers to siRNAs composed of a single strand of RNA that possesses regions of self-complementarity that cause the single strand to fold back upon itself and form a hairpin-like structure with an intramolecular duplexed region containing at least 19 basepairs. Importantly, because they are single-stranded, shRNAs can be readily expressed from single expression cassettes.

The “RNAi oligonucleotide,” “RNAi” or “RNAi agent” preferably mediate RNAi with respect to an endogenous ATG14L or Rubicon gene of a subject. RNAi involves multiple RNA-protein interactions characterized by four major steps: assembly of siRNA with the RNA-induced silencing complex (RISC), activation of the RISC, target recognition and target cleavage. Therefore, identifying siRNA-specific features likely to contribute to efficient processing at each step is beneficial efficient RNAi. Reynolds et al. provide methods for identifying such features (A. Reynolds et al., “Rational siRNA design for RNA interference,” Nature Biotechnology 22(3), March 2004). In that study, eight characteristics associated with siRNA functionality were identified: low G/C content, a bias towards low internal stability at the sense strand 3′-terminus, lack of inverted repeats, and sense strand base preferences (positions 3, 10, 13 and 19). Further analyses revealed that application of an algorithm incorporating all eight criteria significantly improves potent siRNA selection. siRNA sequences that contain internal repeats or palindromes may form internal fold-back structures. These hairpin-like structures may exist in equilibrium with the duplex form, reducing the effective concentration and silencing potential of the siRNA. The relative stability and propensity to form internal hairpins can be estimated by the predicted melting temperatures (T_{M}). Sequences with high T_{M} values would favor internal hairpin structures.

siRNA can be used either ex vivo or in vivo, making it useful in both research and therapeutic settings. Unlike in other antisense technologies, the RNA used in the siRNA technique has a region with double-stranded structure that is made identical to a portion of the target gene, thus making inhibition sequence-specific. Double-stranded RNA-mediated inhibition has advantages both in the stability of the material to be delivered and the concentration required for effective inhibition.

The extent to which there is loss of function of the target gene can be titrated using the dose of double stranded RNA delivered. A reduction or loss of gene expression in at least 99% of targeted cells has been shown. See, e.g., U.S. Pat. No. 6,506,559. Lower doses of injected material and longer times after administration of siRNA may result in inhibition in a smaller fraction of cells. Quantization of gene expression in a cell show similar amounts of inhibition at the level of accumulation of target mRNA or translation of target protein.

The RNA used in this technique can comprise one or more strands of polymerized ribonucleotides, and modification can be made to the sugar-phosphate backbone as disclosed above. The double-stranded structure is often formed using either a single self-complementary RNA strand (hairpin) or two complementary RNA strands. RNA containing a nucleotide sequences identical to a portion of the target gene is preferred for inhibition, although sequences with insertions, deletions, and single point mutations relative to the target sequence can also be used for inhibition. Sequence identity may be optimized using alignment algorithms known in the art and through calculating the percent difference between the nucleotide sequences. The duplex region of the RNA could also be described in functional terms as a nucleotide sequence that is capable of hybridizing with a portion of the target gene transcript.

Generally, the “RNAi oligonucleotide,” “RNAi” or “RNAi agent” of the instant invention include a region of sufficient complementarity to a targeted RNA (ATG14L or Rubicon RNA), and are of sufficient length in terms of nucleotides, such that the RNAi oligonucleotide, RNAi or RNAi agent, or a fragment thereof, can mediate down regulation of the ATG14L or Rubicon gene. It is not necessary that there be perfect complementarity between the RNAi oligonucleotide, RNAi, or RNAi agent and the target, but the correspondence must be sufficient to enable the RNAi oligonucleotide, RNAi, or RNAi agent, or a cleavage product thereof, to direct sequence specific silencing, e.g., by RNAi cleavage of an ATG14L or Rubicon RNA.

The terms “complementary” and “complementarity” refer to the extent to which a nucleic acid can form hydrogen bond(s) with another nucleic acid sequence by either traditional Watson-Crick hydrogen bonding or by other non-traditional types. The term is used herein to indicate a sufficient degree of hydrogen bonding such that stable and specific binding occurs between a compound of the invention and a target nucleic acid molecule, e.g., an ATG14L or Rubicon mRNA molecule. Specific binding requires a sufficient degree of hydrogen bonding to avoid non-specific binding of the nucleic acid to non-target nucleic acid sequences under conditions in which specific binding is desired, i.e., under physiological conditions in the case of in vivo assays or therapeutic treatment, or in the case of in vitro assays, under conditions in which the assays are performed. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementarity respectively).

siRNA can often be a more effective therapeutic tool than other types of gene suppression due to siRNA's potent gene inhibition and ability to target receptors with a specificity can reach down to the level of single-nucleotide polymorphisms. Such specificity generally results in fewer side effects than is seen in conventional therapies, because other genes are not be affected by application of a sufficiently sequence-specific siRNA.

There are multiple ways to deliver siRNA to the appropriate target. Standard transfection techniques may be used, in which siRNA duplexes are incubated with cells of interest and then processed using standard commercially available kits. Electroporation techniques of transfection may also be appropriate. Cells or organisms can be soaked in a solution of the siRNA, allowing the natural uptake processes of the cells or organism to introduce the siRNA into the system. Viral constructs packaged into a viral particle would both introduce the siRNA into the cell line or organism and also initiate transcription through the expression construct. Other methods known in the art for introducing nucleic acids to cells may also be used, including lipid-mediated carrier transport, chemical-mediated transport, such as calcium phosphate, and the like.

For therapeutic uses, tissue-targeted nanoparticles may serve as a delivery vehicle for siRNA. These nanoparticles carry the siRNA exposed on the surface, which is then available to bind to the target gene to be silenced (Schiffelers, et al., Nucleic Acids Research (2004) 32(19):e149). These nanoparticles may be introduced into the cells or organisms using the above described techniques already known in the art. Designing the appropriate nanoparticles for a particular illness is a matter of determining the appropriate targets for the particular disease. In the case of IPAH, the present invention has already revealed potential targets for this powerful therapy.

DNAzyme

“DNAzyme” as used herein, refers to an enzymatic nucleic acid molecule that does not require the presence of a 2′-OH group within its own nucleic acid sequence for activity. In particular embodiments, the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. Non-limiting examples of DNAzymes are generally reviewed in Usman et al., U.S. Pat. No. 6,159,714, which is herein incorporated by reference in its entirety including the drawings; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection as generally described in Joyce et al., U.S. Pat. No. 5,807,718 and Santoro et al., supra. Additional DNAzyme motifs can be selected for using techniques similar to those described in these references.

### Gene Therapy

In one embodiment, nucleic acids comprising sequences encoding ATG14L or Rubicon protein, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an ATG14L or Rubicon polypeptide or functional fragment thereof, said nucleic acid sequences being part of expression vectors that express the ATG14L or Rubicon polypeptide or functional fragments thereof in a suitable host. In particular, such nucleic acid sequences have promoters operably linked to the ATG14L or Rubicon coding region, said promoter being inducible or constitutive, and, optionally, tissue-specific.

Delivery of nucleic acid into a subject or cell may be either direct, in which case the subject or cell is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

The nucleic acid may be directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. (1987); 262:4429-4432) (which can be used to target cell types specifically expressing the receptors), etc. The nucleic acid-ligand complexes can also be formed in which the ligand comprises a fusogenic viral peptide to disrupt endosomes, allowing the nucleic acid to avoid lysosomal degradation. In addition, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA (1989); 86:8932-8935; Zijlstra et al., Nature (1989); 342:435-438).

In a specific embodiment, a viral vector that contains nucleic acid encoding an ATG14L or Rubicon polypeptide or a functional fragment thereof may be used. For example, a retroviral vector can be used (see Miller et al., Meth. Enzymol. (1993); 217:581-599). These retroviral vectors contain the components necessary for the correct packaging of the viral genome and integration into the host cell DNA. More detail about retroviral vectors can be found in Boesen et al., Biotherapy (1994); 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. (1994); 93:644-651; Kiem et al., Blood (1994); 83:1467-1473; Salmons and Gunzberg, Human Gene Therapy (1993); 4:129-141; and Grossman and Wilson, Curr. Opin. in Genetics and Devel. (1993); 3:110-114.

Adenoviruses are especially attractive vehicles for delivering genes. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) may also be used (Walsh et al., Proc. Soc. Exp. Biol. Med. 204:289-300 (1993); U.S. Pat. No. 5,436,146).

Vectors that can be used in gene therapy are discussed below in details at the “VECTORS” section.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

The nucleic acid can be introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

Recombinant cells can also be used in gene therapy, where nucleic acid sequences encoding an ATG14L or Rubicon or functional fragment thereof, are introduced into the cells such that they are expressible by the cells or their progeny, and the recombinant cells are then administered in vivo for therapeutic effect. For example, stem or progenitor cells can be used. Any stem and/or progenitor cells which can be isolated and maintained in vitro can potentially be used (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

The compounds or pharmaceutical compositions of the invention are preferably tested in vitro, and then in vivo for the desired therapeutic activity, prior to use in humans. For example, in vitro assays to demonstrate the therapeutic utility of a compound or pharmaceutical composition include, the effect of a compound on a cell or a patient tissue sample. The effect of the compound or composition on the cell and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

### Vectors

The terms “vector” and “expression vector” mean the vehicle by which a DNA or RNA sequence (e.g., a foreign gene) can be introduced into a host cell, so as to transform the host and promote expression (e.g., transcription and translation) of the introduced sequence. Vectors include plasmids, phages, viruses, etc.; they are discussed in greater detail below. A “therapeutic vector” as used herein refers to a vector which is acceptable for administration to an animal, and particularly to a human.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET plasmids (Invitrogen, San Diego, Calif.), pcDNA3 plasmids (Invitrogen), pREP plasmids (Invitrogen), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

Suitable vectors include viruses, such as adenoviruses, adeno-associated virus (AAV), vaccinia, herpesviruses, baculoviruses and retroviruses, parvovirus, lentivirus, bacteriophages, cosmids, plasmids, fungal vectors, naked DNA, DNA lipid complexes, and other recombination vehicles typically used in the art which have been described for expression in a variety of eukaryotic and prokaryotic hosts, and may be used for gene therapy as well as for simple protein expression.

Viral vectors, especially adenoviral vectors can be complexed with a cationic amphiphile, such as a cationic lipid, polyL-lysine (PLL), and diethylaminoethyldextran (DELAE-dextran), which provide increased efficiency of viral infection of target cells (See, e.g., PCT/US97/21496 filed Nov. 20, 1997, incorporated herein by reference). AAV vectors, such as those disclosed in U.S. Pat. Nos. 5,139,941, 5,252,479 and 5,753,500 and PCT publication WO 97/09441, the disclosures of which are incorporated herein, are also useful since these vectors integrate into host chromosomes, with a minimal need for repeat administration of vector. For a review of viral vectors in gene therapy, see McConnell et al., 2004, Hum Gene Ther. 15(11):1022-33; Mccarty et al., 2004, Arum Rev Genet. 38:819-45; Mah et al., 2002, Clin. Pharmacokinet. 41(12):901-11; Scott et al., 2002, Neuromuscul. Disord. 12(Suppl 1):S23-9. In addition, see U.S. Pat. No. 5,670,488. Beck et al., 2004, Curr Gene Ther. 4(4): 457-67, specifically describe gene therapy in cardiovascular cells.

### Treatment of Cancers, Neurodegenerative Diseases, Age-Related Disease and Heart Disease

The present invention further provides a method of treating cancer in a subject. In one non-limiting embodiment, the method of treating cancer comprises administering to the subject a therapeutically effective amount of an agent which increases or enhances the biological activity of ATG14L. In another non-limiting embodiment, the method of treating cancer comprises administering to the subject a therapeutically effective amount of an agent which decreases or inhibits the biological activity of Rubicon. For example, the cancer is a cancer associated with an autophagy defect, breast cancer, liver cancer, and lung cancer.

The terms “cancer” as used herein, refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, gastric cancer, melanoma, and various types of head and neck cancer. Dysregulation of angiogenesis can lead to many disorders that can be treated by compositions and methods of the invention. These disorders include both non-neoplastic and neoplastic conditions. Neoplastics include but are not limited those described above. Non-neoplastic disorders include but are not limited to undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

Also provided is a method of treating a neurodegenerative disease in a subject. In one embodiment, the method of treating a neurodegenerative disease comprises administering to the subject a therapeutically effective amount of an agent which increases or enhances the biological activity of ATG14L. In another embodiment, the method of treating a neurodegenerative disease comprises administering to the subject a therapeutically effective amount of an agent which decreases or inhibits the biological activity of Rubicon. For example, the neurodegenerative disease is a neurodegenerative disease associated with an autophagy defect, Parkinson's disease, Huntington's disease, Alzheimer's disease, or Amyotrophic Lateral Sclerosis (ALS).

The term “neurodegenerative diseases” as used herein, includes, but are not limited to, demyelinating diseases, such as multiple sclerosis and acute transverse myelitis; extrapyramidal and cerebellar disorders such as lesions of the corticospinal system; disorders of the basal ganglia or cerebellar disorders; hyperkinetic movement disorders such as Huntington's disease, Huntington's Chorea and senile chorea; drug-induced movement disorders, such as those induced by drugs which block CNS dopamine receptors; hypokinetic movement disorders, such as Parkinson's disease; Progressive supranucleo palsy; Cerebellar and Spinocerebellar Disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and MachadoJoseph)); and systemic disorders (Refsum's disease, abetalipoprotemia, ataxia, telangiectasia, and mitochondrial multi-system disorder); demyelinating core disorders, such as multiple sclerosis, acute transverse myelitis; disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Amyotrophic Lateral Sclerosis (ALS), Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; and Dementia pugilistica, or any subset thereof.

Furthermore, the present invention provides a method of treating an inflammatory disease in a subject. In one embodiment, the method of treating an inflammatory disease comprises administering to the subject a therapeutically effective amount of an agent which increases or enhances the biological activity of ATG14L. In another embodiment, the method of treating an inflammatory disease comprises administering to the subject therapeutically effective amount of an agent which decreases or inhibits the biological activity Rubicon. For example, the inflammatory disease is an inflammatory disease associated with an autophagy defect, or Crohn's disease.

Inflammatory diseases can arise where there is an inflammation of the body tissue. The term “inflammatory diseases” as used herein, includes, but are not limited to, local inflammatory responses and systemic inflammation. Examples of such diseases include: organ transplant rejection; reoxygenation injury resulting from organ transplantation (Grupp et al. J. Mol. Cell. Cardiol. (1999); 31:297-303) including, but not limited to, transplantation of the following organs: heart, lung, liver and kidney; chronic inflammatory diseases of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung diseases such as asthma, adult respiratory distress syndrome, and chronic obstructive airway disease; inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; tuberculosis; leprosy; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer s disease, infectious meningitis, encephalomyelitis, Parkinsonss disease, Huntington's disease, amyotrophic lateral sclerosis and viral or autoimmune encephalitis; autoimmune diseases including Type I and Type II diabetes mellitus; diabetic complications, including, but not limited to, diabetic cataract, glaucoma, retinopathy, nephropathy, such as microaluminuria and progressive diabetic nephropathy, polyneuropathy, gangrene of the feet, atherosclerotic coronary arterial disease, peripheral arterial disease, nonketotic hyperglycemic-hyperosmolar coma, mononeuropathies, autonomic neuropathy, foot ulcers, joint problems, and a skin or mucous membrane complication, such as an infection, a shin spot, a candidal infection or necrobiosis lipoidica diabeticorum; immune-complex vasculitis, systemic lupus erythematosus (SLE); inflammatory diseases of the heart such as cardiomyopathy, ischemic heart disease hypercholesterolemia, and atherosclerosis; as well as various other diseases that can have significant inflammatory components, including preeclampsia; chronic liver failure, brain and spinal cord trauma, and cancer. The inflammatory disease can also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

The present invention also provides a method of treating an age-related disease in a subject. In one embodiment, the method of treating an age-related disease comprises administering to the subject a therapeutically effective amount of an agent which increases or enhances the biological activity of ATG14L. In another embodiment, the method of treating an age-related disease comprises administering to the subject a therapeutically effective amount of an agent which decreases or inhibits the biological activity of Rubicon. For example, the age-related disease is associated with an autophagy defect.

The term “age-related disease” is used herein to encompass all types of diseases associated with normal as well as premature cellular and organism aging, including without limitation atherosclerosis, emphysema and respiratory distress syndrome, cancer, stomach and intestinal ulcers, degenerative diseases of the brain, liver, lung, and intestine, for example, neurodegenerative diseases such as Alzheimer's disease, Huntington's Disease, and Parkinson's disease.

Also provided is a method of treating heart disease in a subject. In one embodiment, the method comprises administering to the subject a therapeutically effective amount of an agent which modulates the biological activity of ATG14L. In one embodiment, the agent increases the biological activity of ATG14L, during ischemia. In another embodiment, the agent decreases the biological activity of ATG14L, during reperfusion. In another embodiment, the method of treating heart diseases in a subject comprises administering to the subject a therapeutically effective amount of an agent which modulates the biological activity of Rubicon. In one embodiment, the agent decreases the biological activity of Rubicon, during ischemia. In another embodiment, the agent increases the biological activity of Rubicon, during reperfusion.

A “disorder” or “disease” as used herein is any condition that would benefit from treatment with a substance/molecule or method of the invention. This includes chronic and acute disorders or diseases including those pathological conditions which predispose the mammal to the disorder in question. Non-limiting examples of disorders to be treated herein include malignant and benign tumors; carcinoma, blastoma, and sarcoma.

As used herein, a “treating” or “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies are used to delay development of a disease or disorder.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

As used herein, a “therapeutically effective amount” refers to an amount effective, at dosages and/or for periods of time necessary, to achieve the desired therapeutic or prophylactic result. In one non-limiting embodiment, a therapeutically effective amount of an agent that increases or enhances the biological activity of ATG14L refers to an amount effective at dosages and/or periods of time necessary to increase or enhance the biological activity of ATG14L by at least 10%, preferably 20%, more preferably 30%. In one non-limiting embodiment, a therapeutically effective amount of an agent that increases or enhances the biological activity of Rubicon refers to an amount effective at dosages and/or periods of time necessary to increase or enhance the biological activity of Rubicon by at least 10%, preferably 20%, more preferably 30%. In one non-limiting embodiment, a therapeutically effective amount of an agent that decreases or inhibits the biological activity of ATG14L refers to an amount effective at dosages and/or periods of time necessary to decrease or inhibit the biological activity of ATG14L by at least 10%, preferably 20%, more preferably 30%. In one non-limiting embodiment, a therapeutically effective amount of an agent that decreases or inhibits the biological activity of Rubicon refers to an amount effective at dosages and/or periods of time necessary to decrease or inhibit the biological activity of Rubicon by at least 10%, preferably 20%, more preferably 30%. The biological activity of ATG14L includes, but is not limited to, ability of ATG14L to bind to Beclin 1, to bind to a class III PI3K/Vps34-Beclin 1 autophagic complex, to increase class III PI3K/Vps34 kinase activity, and/or to increase autophagic activity. The biological activity of Rubicon includes, but is not limited to, ability of Rubicon to bind to a class III PI3K/Vps34-Beclin 1 autophagic complex, to decrease class III PI3K/Vps34 kinase activity, and/or to decrease autophagic activity.

### Method for Monitoring Autophagy or Autophagic Activity

The present invention provides a method of modulating autophagic activity in a cell. In one non-limiting embodiment, a method of increasing autophagic activity in a cell comprises administering to the cell an effective amount of an agent which increases or enhances the biological activity of ATG14L. In another non-limiting embodiment, a method of increasing autophagic activity in a cell comprises administering to the cell an effective amount of an agent which decreases or inhibits the biological activity of Rubicon. In one non-limiting embodiment, a method of decreasing autophagic activity in a cell comprises administering to the cell an effective amount of an agent which increases or enhances the biological activity of Rubicon. In another non-limiting embodiment, a method of decreasing autophagic activity in a cell comprises administering to the cell an effective amount of an agent which decreases or inhibits the biological activity of ATG14L.

The most standard method to monitor autophagy or autophagic activity is conventional electron microscopy (EM). Quantification of autophagic activity by EM is possible. In yeast, accumulation of undigested autophagic bodies in the vacuole can be seen simply by light microscopy. It is achieved by using vacuolar protease deficient strains or by treatment with protease inhibitors such as phenylmethanesulfonyl fluoride (PMSF) to inhibit the degradation of autophagic bodies (Takeshige, Baba, Tsuboi, Noda, & Ohsumi, 1992). A fluorescent compound, monodansylcadaverine (MDC) has been proposed as a tracer for autophagic vacuoles (Biederbick, Kern, & Elsasser, 1995). In addition, some biochemical methods have been utilized to measure autophagic activity. In contrast to the ubiquitin-proteasome system which predominantly degrades short-lived proteins, autophagy is believed to account for the majority of degradation of long-lived proteins (Mortimore & Poso, 1987). Therefore, measurement of bulk degradation of long-lived proteins is often used to monitor autophagic activity. Likewise, autophagic activity is often presented as the difference between cells treated with and without 3-methyladenine (3-MA), therefore, 3-MA can be used to monitor autophagy. Another method to assess autophagic activity is measuring the delivery of cytosolic material to lysosomes. This approach is the most successful using yeast cells (Nada, Matsuura, Wada, & Ohsumi, 1995).

Recently, the molecular basis of autophagosome formation has been extensively studied using yeast cells; these studies have provided useful marker proteins for autophagosomes. Importantly, most of these proteins are conserved in mammals. These proteins are used for monitoring autophagic activity. LC3 is a general marker for autophagic membranes. The localization of LC3 is easily examined by generating chimeric proteins fused with green fluorescent protein (GFP) or its derivatives. Examination of GFP-LC3 localization is a very simple method, which requires only a high-resolution fluorescence microscope. In one non-limiting embodiment, a double-tagged LC3 reporter, mCherry-GFP-LC3 is used to monitor autophagic activity. LC3 is processed from a full-length protein (LC3I) to a cleaved and lipidated form (LC3II) during autophagy. The abundance of LC3II reflects the level of autophagy and LC3-II has been used as a specific marker for autophagy. The amount of LC3II, the LC3II/LC3I ratio, or the conversion rate of LC3I to LC3II correlates with the number of autophagosomes (Mizushima N., Int J Biochem Cell Biol (2004); 36:2491-502). Therefore, an increased LC3I/II ratio or decreased LC3II/I ratio suggests up-regulated/increased autophagic activity, and a decreased LC3I/II ratio or increased LC3II/I ratio suggests down-regulated/decreased autophagic activity. An increased conversion rate of LC1 to LC3 μl suggests up-regulated/increased autophagic activity, and a decreased conversion rate of LC1 to LC3II suggests down-regulated/decreased autophagic activity.

P62 protein can also be used as an autophagic marker. A decreased level of p62/SQSTM1 suggests an up-regulated/increased autophagic activity, and an increased level of p62/SQSTM1 suggests a down-regulated/decreased autophagic activity. P62 protein, also called sequestosome 1 (SQSTM1), is commonly found in inclusion bodies containing polyubiquitinated protein aggregates. The p62 protein level increases after oxygen radical stress. Polymerization of the polyubiquitin-binding protein p62/SQSTM1 yields protein bodies that either reside free in the cytosol and nucleus or occur within autophagosomes and lysosomal structures. Inhibition of autophagy led to an increase in the size and number of p62 bodies and p62 protein levels. Studies have shown that LC3 co-localized with p62 bodies and co-immunoprecipitated with p62, suggesting that these two proteins participate in the same complexes. The depletion of p62 inhibited recruitment of LC3 to autophagosomes under starvation conditions. Reduction of p62 protein levels or interference with p62 function significantly increased cell death that was induced by the expression of mutant huntingtin. Therefore, the polyubiquitin-binding and homopolymerizing p62 protein may, via LC3, be involved in linking polyubiquitinated protein aggregates to the autophagic machinery, facilitating the clearance of such aggregates and, thereby, contributing to reduced toxicity of mutant huntingtin expression.

### Method for Measuring Class III PI3K/Vps34 Kinase Activity

The present invention provides a method of modulating class III PI3K/Vps34 kinase activity in a cell. In one non-limiting embodiment, a method of increasing class III PI3K/Vps34 kinase activity in a cell comprises administering to the cell an effective amount of an agent which increases or enhances the biological activity of ATG14L. In another non-limiting embodiment, a method of increasing class III PI3K/Vps34 kinase activity in a cell comprises administering to the cell an effective amount of an agent which decreases or inhibits the biological activity of Rubicon. In one non-limiting embodiment, a method of decreasing class III PI3K/Vps34 kinase activity in a cell comprises administering to the cell an effective amount of an agent which increases or enhances the biological activity of Rubicon. In another non-limiting embodiment, a method of decreasing class III PI3K/Vps34 kinase activity in a cell comprises administering to the cell an effective amount of an agent which decreases or inhibits the biological activity of ATG14L.

General methods for measuring kinase activity can be used to measure Class III PI3K/Vps34 kinase activity. Methods for measuring the phosphorylation state of the kinases have traditionally relied on radioactive means, for example, ³²P or ³³P-gammaphosphate incorporation or assays employing labeled antibodies. Phosphorylated tyrosine residues, for example, can be detected either by immunoprecipitation or blotting using a radiolabeled antiphosphotyrosine antibody or using filter binding to trap radiolabeled products. In whole cells, incorporation of radioactive phosphate from the media into protein is monitored. In crude extracts, the extent of ³²P incorporation is measured following the addition of [gamma-³²P]ATP and activators of specific kinases. In one non-limiting embodiment, the PtdIns3K/Vps34 kinase activity is measured by monitoring the incorporation of radioactive-³²P-ATP into class III PtdIns3K/Vps34.

A standard enzyme-linked immunosorbent assay (ELISA) format can be used for measuring kinase activity. This method utilizes purified heterologous substrate protein or synthetic substrate peptides anchored to a microtiter plate. After exposure of the substrate molecule to a sample containing the appropriate kinase, the level of phosphorylation is evaluated with antiphosphotyrosine antibodies to quantitate the amount of phosphorylated protein bound to the plate. In this method, kinase activity is determined after any unbound antibody is removed from the plate.

### Pharmaceutical Compositions

The present invention provides a pharmaceutical composition which comprises (i) purified ATG14L protein, ATG14L polypeptide, anti-ATG14L antibody, ATG14L agonist, ATG14L antagonist, or RNAi (or functional fragment thereof), or (ii) purified Rubicon protein, Rubicon polypeptide, anti-Rubicon antibody, Rubicon agonist, Rubicon antagonist, or RNAi (or functional fragment thereof), and a pharmaceutically acceptable carrier. A functional fragment of an ATG14L or Rubicon protein, anti-ATG14L or anti-Rubicon antibody or RNAi agent of this invention is one that agonizes or antagonizes activity of the native ATG14L or Rubicon protein, depending on its intended function. It is particularly contemplated that certain functional fragments of ATG14L or Rubicon proteins will act as competitive inhibitors of the native proteins.

As used herein, the term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art. Methods for purification are well-known in the art.

### Methods of Screening

The present invention provides a method of screening for compounds that modulate a property of a class III PI3K/Vps34-Beclin-1 autophagic complex, by enhancing or inhibiting the biological activity of either ATG14L or Rubicon. This method comprises observing a change in a property of the class III PI3K/Vps34-Beclin-1 autophagic complex contacted with a candidate compound. For example, the class PI3K/Vps34 kinase activity and autophagic activity can be altered in the presence of a test compound. In particular, the altered autophagic activity can be shown by altered LC3I/LC3II level or altered conversion rate of LC3I to LC3II, and altered p62/SQSTM1 level.

The present invention provides a method of screening for a compound that binds to an ATG14L or Rubicon polypeptide. The method comprises providing an ATG14L or Rubicon polypeptide; contacting a candidate compound with the ATG14 or Rubicon polypeptide; and determining whether the candidate compound has bound to the ATG14L or Rubicon polypeptide.

Also provided is a method of screening for a compound that modulates biological activity of ATG14L or Rubicon. The method comprises contacting ATG14L or Rubicon with a candidate compound in a manner wherein the candidate compound interacts with ATG14L or Rubicon; (b) evaluating the biological activity of ATG14L or Rubicon; and (c) identifying a candidate compound as a modulator of biological activity of ATG14L or Rubicon based upon observing increased or decreased biological activity of Rubicon in the presence of the candidate compound compared to a control sample.

The term “modulate” as used herein, is meant that the expression of the gene, or level of a RNA molecule or equivalent RNA molecules encoding one or more proteins or protein subunits, or activity of one or more proteins or protein subunits is up-regulated/increased or down-regulated/decreased, such that expression, level, or activity is greater than or less than that observed in the absence of the modulator. In this context, the term “modulate” includes both promotion and inhibition.

The term “screening” used herein, refers to a process of testing one or a plurality of compounds (including a library of compounds) for some activity. A “screen” is a test system for screening. Screens can be primary, i.e., an initial selection process, or secondary, e.g., to confirm that a compound selected in a primary screen (such as a binding assay) functions as desired (such as in a signal transduction assay). Screening permits the more rapid elimination of irrelevant or non-functional compounds, and thus selection of more relevant compounds for further testing and development. “High throughput screening” involves the automation and robotization of screening systems to rapidly screen a large number of compounds for a desired activity. Screens are discussed in greater detail below.

Screening Assays

The present invention provides various screening assays for compounds that modulate the functionality or a property of a class III phophatidylinositol 3′-kinase (PI3K)/Vps34-Beclin-1 autophagic complex. The present invention contemplates screens for small molecule compounds, including peptides and peptidomimetics, and including receptor ligand analogs and mimics, as well as screens for natural compounds that bind to and modulate a property of a class III phophatidylinositol 3′-kinase (PI3K)/Vps34-Beclin-1 autophagic complex in vitro. A property of a class III phophatidylinositol 3′-kinase (PI3K)/Vps34-Beclin-1 autophagic complex includes, but not limited to, PI3K/Vps34 kinase activity and autophagic activity, for example, the LC3I/LC3II level or the conversion rate of LC3I to LC3II, and p62/SQSTM1 level.

The present invention also provides various screening assays for screening for compounds that enhance or inhibit the biological activity or expression of ATG14L or Rubicon based on PI3K/Vps34 activity as described above.

The present invention contemplates screens for, as examples and not by way of limitation, synthetic small molecule agents, chemical compounds, chemical complexes, and salts thereof as well as screens for natural products, such as plant extracts or materials obtained from fermentation broths. Other molecules that can be identified using the screens of the invention include opioids, opiates, narcotics, proteins and peptide fragments, peptides, nucleic acids and oligonucleotides, carbohydrates, phospholipids and other lipid derivatives, steroids and steroid derivatives, prostaglandins and related arachadonic acid derivatives, etc.

One approach to identifying such compounds uses recombinant bacteriophage to produce large libraries. Using the “phage method” (Scott and Smith, Science, 1990, 249:386-390; Cwirla, et al., 1990, Proc. Natl. Acad. Sci. USA, 87:6378-6382; Devlin et al., 1990, Science 49:404-406), very large libraries can be constructed (10⁶-10⁸ chemical entities). A second approach uses primarily chemical methods, of which the Geysen method (Geysen et al., 1986, Molecular Immunology 23:709-715; Geysen et al., 1987, J. Immunologic Method 102:259-274) and the method of Fodor et al. (1991, Science 251:767-773) are examples. Furka et al. (1988, 14th International Congress of Biochemistry, Volume 5, Abstract FR:013; Furka, 1991, Int J. Peptide Protein Res. 37:487-493) and U.S. Pat. Nos. 4,631,211 and 5,010,175 describe methods to produce a mixture of peptides that can be tested as agonists or antagonists.

In another aspect, synthetic combinatorial libraries (Needels et al., 1993, Proc. Natl. Acad. Sci. USA, 90:10700-4; Ohlmeyer et al., 1993, Proc. Natl. Acad. Sci. USA 90:10922-10926; PCT Publication Nos. WO 92/00252 and WO 94/28028) and the like can be used to screen for compounds according to the present invention.

Test compounds may be screened from large libraries of synthetic or natural compounds. Numerous means are currently used for random and directed synthesis of saccharide-, peptide-, and nucleic acid-based compounds. Synthetic compound libraries are commercially available, for example, from Maybridge Chemical Co. (Trevillet, Cornwall, UK), Comgenex (Princeton, N.J.), and Microsource (New Milford, Conn.). A rare chemical library is available from Aldrich (Milwaukee, Wis.). Alternatively, libraries of natural compounds in the form of bacterial, fungal, plant and animal tracts are available from, e.g., Pan Laboratories (Bothell, Wash.) or MycoSearch (NC), or are readily producible. Additionally, natural and synthetically produced libraries and compounds are readily modified through conventional chemical, physical, and biochemical means (Blondelle et al., 1996, Tib Tech 14:60).

High Throughput Screens

Agents according to the invention may be identified by screening in high-throughput assays, including without limitation cell-based or cell-free assays. It will be appreciated by those skilled in the art that different types of assays can be used to detect different types of agents. Several methods of automated assays have been developed in recent years so as to permit screening of tens of thousands of compounds in a short period of time e.g., using a 96-well format). For literature references see, e.g., Beggs et al., 1999, J. Biol. Screening 4(3); Renate de Wit et al., 1998, J. Biol. Screening, 3(4); Fox et al., 1999, J. Biol. Screening 4(4); Boyd et al., 1996, Clin. Chem. 42:1901-10; Broach et al., 1996, Nature 384(Supp.):14-16; Cusack et al., 1993, J. Rec. Res. 13:123-134; U.S. Pat. Nos. 4,980,281 and 5,876,951; PCT Publication Nos. WO 97/45730, WO 97/14812, and WO 97/10502. Such high-throughput screening methods are particularly preferred.

### Recruitment of ATG12, ATG5, and ATG6

The present invention provides a method of increasing the recruitment of ATG12, ATG5, and ATG6 to a class III PI3K/Vps34-Beclin-1 autophagic complex in a cell, which comprises administering to the cell an effective amount of an agent which increases or enhances the biological activity of ATG14L. The present invention also provides a method of decreasing the recruitment of ATG12, ATG5, and ATG6 to a PI3K/Vps34-Beclin-1 autophagic complex in a cell, which comprises administering to the cell an effective amount of an agent which decreases or inhibits the biological activity of ATG14L. The biological activity of ATG14L includes, but is not limited to, ability of ATG14L to bind to Beclin 1, to bind to a class III PI3K/Vps34-Beclin-1 autophagic complex, to increase class III PI3K/Vps34 kinase activity, and/or to increase autophagic activity. The agent that increases or enhances the biological activity of ATG14L includes, but is not limited to, an ATG14L, a functional agonistic fragment thereof, an ATG14L polypeptide equivalent, an ATG14L mimetic compound, a therapeutic vector which comprises a nucleic acid molecule encoding ATG14L protein, a binding enhancer that enhances or prolongs the binding between ATG14L and class III PI3K/Vps34-Beclin 1 autophagic complex, and a binding enhancer that enhances or prolongs the binding between ATG14L and Beclin 1. The agent that decreases or inhibits the biological activity of ATG14L includes, but is not limited to, a functional antagonistic fragment of ATG14L, an anti-ATG14L antibody or fragment thereof such as an intrabody, another agent which inhibits or blocks ATG14L biological activity, and a nucleic acid targeted to the ATG14L gene, such as an antisense nucleic acid, a DNA construct for expression of an antisense RNA, a ribozyme, a DNA construct for expression of a ribozyme, a DNAzyme, and an RNAi agent.

### 6. EXAMPLES

### 6.1 Example 1

### 6.1.1 Results

Identification of ATG14L and Rubicon as novel Beclin 1-binding proteins in transgenic mice expressing functional fusion protein Beclin 1-EGFP

Affinity purification of tagged proteins expressed in cultured cells is commonly used for identifying protein-binding partners. A potential disadvantage of this approach is competition from the endogenous protein with the exogenously tagged protein for the binding of the same partners, preventing efficient capture of the binding proteins. In addition, it is difficult to assess whether the identified binding proteins are cell/tissue-specific. To address these issues, generated BAC (Bacterial Artificial Chromosome)-mediated transgenic mice expressing a fusion protein of Beclin 1 and enhanced green fluorescence protein (Beclin 1-EGFP) was generated by insertion of the EGFP cDNA sequence at the C-terminus of beclin 1 (FIG. 9A). These beclin 1-EGFP transgenic mice are expected to produce Beclin 1-EGFP under the transcriptional control of endogenous regulatory elements (Heintz, N. Nat Rev Neurosci (2000; 2, 861; Gong S. et al., Genome Res. (2002); 12, 1992-1998). Western blot analysis of mouse tissue extracts with anti-Beclin 1 antibody revealed the expression of Beclin 1-EGFP protein at 90 kD (FIG. 1A). To establish that the Beclin1-EGFP fusion is functional, and to enable biochemical studies of Beclin1 interacting proteins under physiological condition (e.g. in the absence of interference from endogenous Beclin 1), mice in which Beclin 1-EGFP replaced endogenous Beclin 1 expression were generated (FIG. 9B). In such mice (beclin 1^{-/-}; beclin 1-EGFP/+), only the Beclin 1-EGFP fusion protein was detected by anti-Beclin 1 antibody (FIG. 1a). These mice were born at the expected Mendelian ratio (11 out of total 69, chi square test with the value 0.22, P>0.6, df=1) (FIG. 9C) and had no overt anatomic or behavioral phenotypes. Of note, the expression levels of Beclin 1-EGFP was nearly at the endogenous Beclin 1 levels (FIG. 1A, middle lane). This result suggested that BAC-mediated expression of the Beclin1-GFP fusion protein completely rescues the embryonic lethality observed in beclin1 knockout animals (beclin 1^{-/-}) (Yue Z. et al., Proceedings of the National Academy of Sciences of the United States of America (2003); 100, 15077-82).

Using the “rescued mice” (beclin 1^{-/-}; beclin 1-GFP/+), Beclin 1-EGFP protein were affinity-purified from liver, brain (FIG. 1B) and thymus. Beclin 1-EGFP (˜90 kD) and its associated proteins were separated on SDS-PAGE, and the components were identified using mass-spectrometry (FIG. 10). Compared to the wild type (control) mice, the “rescued mice” contained at least six additional and readily detectable protein bands common to liver and brain (FIG. 1B) which included the tagged protein Beclin 1-EGFP (#4), three previously reported Beclin 1-binding proteins Vps15/p150 (#1)²², Vps34/PtdIns3K (#3) and UVRAG (#5) (Liang, C. et al. Autophagic and tumour suppressor activity of a novel Beclin1-binding protein UVRAG. Nat Cell Biol (2006); 8, 688-99), and two novel proteins (#2 and 6, asterisks). The first novel protein (gi|27369860) which was named as ATG14L (previously called “BISC”), has 492 amino acids (calculated molecular mass=55 kD) and contains sequence homology to the SMC (Structural Maintenance of Chromosomes) motif, which was predicted to be comprised of two coiled-coil domains (aa 75-95 and aa 148-178) (FIG. 1C). The second novel protein (gi|45708948) which was named as Rubicon (previously called “BIRC”), contains 941 amino acids (105 kD) and has a conserved RUN domain (aa 49-190) near the N-terminus, a cysteine-rich domain near the C-terminus (aa 837-890), and a coiled-coil motif (aa 488-508) in the central region (FIG. 1C). As assayed by Coomassie blue staining of SDS-PAGE, ATG14L and Rubicon that were affinity-isolated via Beclin 1-EGFP were consistently stained at significantly lower intensity than Beclin 1-EGFP, p150, Vps34/PtdIns3K and UVRAG, whereas the staining intensity for the four known proteins are comparable (FIG. 1B). This result suggests at least a stable binding of Beclin 1 to Vps34/PtdIns3K, p150 and UVRAG.

To further examine the specific binding of ATG14L or Rubicon to Beclin 1, mouse cDNAs for ATG14L and Rubicon were cloned and expressed in HEK 293T cells by transfection. From the lysates of the transfected cells, FLAG- or EGFP-tagged ATG14L or Rubicon was co-immunoprecipitated with endogenous Beclin 1 protein (FIG. 1A-E). These data has confirmed that the binding of ATG14L or Rubicon to Beclin 1 is specific. Further studies in transfected cells demonstrated that these tagged ATG14L and Rubicon proteins form a complex with both Beclin1 and Vps34/PtdIns3K (FIG. 11).

Taken together, these results have not only confirmed the previously identified Beclin 1-binding proteins (Vps34/PtdIns3K, p150 and UVRAG), but also discovered two novel proteins ATG14L and Rubicon in complex with Beclin 1-Vps34/PtdIns3K. These results have also shown that the binding of ATG14L or Rubicon to Beclin 1 is not restricted to specific tissues or cells.

Mapping of protein sequence domains that are required for the association of Beclin1 with ATG14L and Rubicon

To dissect the protein sequence domains responsible for the association of Beclin 1 with ATG14L or Rubicon, a series of deletion mutants of Beclin 1, ATG14L and Rubicon were constructed (FIG. 2A) and analyzed for their binary interactions. Beclin 1 contains three distinct functional domains: a Bcl-2-interacting domain (BD, aa 88-150), a coiled-coil domain (CCD, aa 144-269), and the evolutionarily conserved domain (ECD, aa 244-337) (Maiuri, M. C. et al., Embo J (2007); 26, 2527-39; Furuya N. et al., Autophagy (2005); 1, 46-52) (FIG. 2A). To test which domain(s) of Beclin 1 is required for ATG14L- or Rubicon-binding, FLAG-tagged Beclin 1 domains and full-length ATG14L-EGFP (or Rubicon-EGFP) were co-expressed in HEK 293T cells. As assayed by immunoprecipitation with anti-GFP antibody, ATG14L-EGFP was able to pull down FLAG-Beclin 1 CCD domain, but not FLAG-Beclin 1 BD domain (aa 1-150) or ECD domain (aa 244-448) (FIG. 2B). This result suggests that CCD of Beclin 1 is sufficient for ATG14L binding. Interestingly, FLAG-Beclin 1 mutant containing both CCD and ECD domains showed significantly increased binding to ATG14L-EGFP as compared to FLAG-Beclin 1 CCD domain alone (FIG. 2B). In contrast, Rubicon-EGFP did not interact with any single Beclin 1 domain (CCD, BD or ECD) domain. However, Rubicon-EGFP was capable of binding FLAG-Beclin 1 mutant containing both CCD and ECD domains (FIG. 2C).

Similar experiments to map domains of ATG14L or Rubicon that are responsible for the interaction with the Beclin 1 complex were also performed. Results showed that ATG14L-EGFP mutants lacking both CCDs completely lost binding to endogenous Beclin 1 or Vps34/PtdIns3K, as detected with anti-Beclin 1 or anti-Vps34/PtdIns3K antibodies, whereas deletion of the N-terminal CCD1 of ATG14L only reduced the level of binding to endogenous Beclin 1 or Vps34/PtdIns3K (FIG. 2D). These data suggest that both CCD domains of ATG14L are important for the efficient interaction between ATG14L and Beclin 1. Rubicon-EGFP mutants (truncated at either N-terminal RUN domain, C-terminal cysteine-rich domain, or both of them) did not prevent binding of Rubicon to the Beclin1-Vps34/PtdIns3K complex (FIG. 2E). In fact, deletion of these domains of Rubicon led to a significant increase of Rubicon binding to the Beclin 1-Vps34/PtdIns3K complex, as compared to the binding of full-length Rubicon-EGFP to the Beclin 1-Vps34/PtdIns3K complex, suggesting that these domains are not required for and are likely inhibitory to the binding of Rubicon to the Beclin 1-Vps34/PtdIns3K complex. Therefore, the central region of Rubicon containing the CCD domain is important for the binding of Rubicon to the Beclin 1-Vps34/PtdIns3K complex.

ATG14L, Rubicon and UVRAG are found in the same complex with Beclin 1-Vps34/PtdIns3K

Previous studies in yeast have identified two distinct functional Atg6-Vps34 complexes: complex I is associated with Atg14 which is specific for autophagy; whereas complex II, containing Vps38, is involved in endocytic trafficking (Zeng X. et al., J Cell Sci (2006); 119, 259-70). However, mammalian homologues of yeast Atg14 or Vps38 have not yet been found. Careful comparison revealed some sequence homology between ATG14L and yeast Atg14, albeit to a moderate degree (15% identity) (FIG. 12), no significant sequence homology was seen between Rubicon and yeast Vps38 or other yeast ATG proteins.

To investigate whether multiple Beclin 1-Vps34/PtdIns3K complexes exist in mammals, developed anti-ATG14L and anti-Rubicon antibodies were developed and performed immunoprecipitation with these antibodies. The result showed that the anti-ATG14L antibody pulled down endogenous ATG14L, together with Vps34/PtdIns3K (FIG. 13) and Beclin 1, but not Rubicon (FIG. 13). In parallel, anti-Rubicon antibody pulled down endogenous Rubicon, Vps34/PtdIns3K and UVRAG, but not ATG14L (FIG. 13).

To further characterize the protein complex formation between Beclin 1 and its binding partners, a series of gel filtration experiments were performed. Protein complexes were separated according to their sphere size under mild condition and expected to have reduced chance of protein dissociation (as opposed to immunoprecipitation). The cytosolic protein complexes prepared from mouse livers of wild-type (FIG. 3A) and “rescued” mice (FIG. 14) were separated by using size exclusion chromatography. Eighty fractions were collected and analyzed by immunoblotting with anti-ATG14L, anti-Rubicon, anti-Vps34/PtdIns3K, anti-Beclin 1 and anti-UVRAG antibodies. From both wild-type and “rescued” mice, Vps34/PtdIns3K and Beclin 1 protein levels peaked in the fractions of 39-40 (FIG. 3A) (Beclin 1-GFP in “rescued” mice, FIG. 14). Interestingly, ATG14L and Rubicon levels also reached climax in the similar fractions (FIGS. 3A & 14), suggesting that these fractions contain a major species of Beclin 1-Vps34/PtdIns3K complex which could include both ATG14L and Rubicon protein (size>700 kD).

To validate the protein detection by anti-ATG14L or anti-Rubicon antibodies, gel filtration was performed by using cell lysate from stable cell line expressing ATG14L-EGFP (FIG. 15A) or Rubicon-EGFP (FIG. 15B), followed by Western blot analysis with anti-GFP or anti-Beclin 1 antibody. The result from the ATG14L-EGFP stable cell lysate showed that the fractions containing peak levels of Beclin 1 overlapped with that of ATG14L-EGFP (both peak fractions eluted at 39-40; FIG. 15A), confirming the results using anti-ATG14L antibodies for detecting endogenous proteins in the liver (FIG. 3A). Interestingly, the samples prepared from starved ATG14L-EGFP stable cells showed little difference in the distribution pattern for ATG14L or Beclin 1 as compared to that of non-starved cells (FIG. 15A). Moreover, the gel filtration study using lysate from Rubicon-EGFP stably transfected cells also confirmed the results using anti-Rubicon antibodies for detecting endogenous Rubicon protein in mouse liver (FIG. 15B).

To test the possibility that ATG14L and Rubicon are present in separate protein complexes while co-eluted, anti-Rubicon antibody was added to the supernatant of the wild-type brain sample before the gel filtration run. By analyzing the fractions with Western blot using anti-ATG14L antibody, it was found that ATG14L (recognized by anti-ATG14L antibody) co-eluted with the anti-Rubicon antibody bands (labeled by *, ** and ***, FIG. 16), demonstrating the binding of anti-Rubicon antibody to, and therefore the presence of Rubicon in, the ATG14L-containing complex.

Co-immunoprecipitation experiments were also performed by using HEK 293 cells transiently transfected with ATG14L, Rubicon, UVRAG and Beclin 1. It showed that ATG14L-EGFP, not EGFP, was co-immunoprecipitated with FLAG-Rubicon and endogenous Beclin 1 (FIG. 3B) by using anti-GFP antibody. Similarly, Rubicon-EGFP, not EGFP, was co-immunoprecipitated with FLAG-ATG14L and endogenous Beclin 1 by anti-GFP antibody (FIG. 3C). This result demonstrates that ATG14L and Rubicon can stay in the same protein complex, consistent with the co-elusion of these two proteins in gel filtration analysis (FIG. 3A). Moreover, these results suggest that UVRAG also resides in the same protein complex with ATG14L or Rubicon, and that the interaction between UVRAG and Rubicon is significantly enhanced in the presence of Beclin 1 (FIG. 17).

The above results and a previous study on UVRAG (Liang C. et al. Nat Cell Biol (2006); 8, 688-99), suggest that ATG14L, Rubicon, UVRAG, Beclin 1, p150 and Vps34/PtdIns3K can form a large protein complex which is the major Beclin 1-Vps34/PdtIns3K complex in vivo. In addition, it was noted that ATG14L was also eluted in later fractions (peak fractions 53-54) in which Beclin 1, but not Rubicon, was detected (FIG. 3A), suggesting that ATG14L is also associated with smaller protein complex without Rubicon. This observation suggests the existence of multiple Beclin 1-ATG14L complexes. Moreover, it may also help explain the previous results in which ATG14L (likely in the small complex) was co-immunoprecipitated with Beclin 1 in the absence of Rubicon (FIG. 13A).

Reduced Expression of Rubicon Results in Autophagy Deficiency and Over-Expression of ATG14L Enhanced Vps34/PtdIns3K Activity

To characterize the role of ATG14L in autophagy, ATG14L protein levels in NTH 3T3 cells were knocked down by using the small RNA interference (RNAi) approach. As detected with anti-ATG14L antibody, ATG14L protein expression was diminished after transfection with ATG14L RNAi (FIG. 4A). The levels of LC3II which a lipid-conjugated form of LC3 that is normally localized on autophagosomes, was analyzed by Westernblot with anti-LC3 antibody (Kabeya Y. et al. EMBO J. (2000); 19, 5720-5728; Ichimura Y. et al., Nature (2000); 408, 488-492; Kabeya Y. et al., J Cell Sci (2004); 117, 2805-2812). The result showed that LC3II was significantly increased in ATG14L RNAi-transfected cells as compared to control RNAi-transfected cells (FIG. 4A, left panel). Similar increase in LC3II levels was also observed with reduced levels of Beclin 1 (FIG. 4A, left panel). The change in LC3II levels suggests an alteration in autophagic activity upon knocking down ATG14L protein levels. However, increased levels of LC3II can be correlated with either enhanced biogenesis of autophagosomes or reduced overall autophagy degradation (Kabeya Y. et al. EMBO J. (2000); 19, 5720-5728). Therefore, to further address the effect of knocking down ATG14L protein levels on autophagic activity, levels of p62/SQSTM1, a known autophagy substrate which is normally accumulated upon impairment of autophagy, were examined by immunoblot analysis with anti-p62/SQSTM1 antibody (Wang Q. J. et al., J Neurosci (2006); 26, 8057-68; Yue Z. Autophagy (2007); 3, 139-41; Komatsu M. et al., Cell (2007); 131, 1149). Similar to Beclin 1 RNAi, ATG14L RNAi transfection resulted in increased p62/SQSTM1 protein levels, as compared to control RNAi transfection (FIG. 4A, left panel). Furthermore, under conditions of nutrient starvation, knock-down of Beclin 1 or ATG14L protein levels with RNAi also caused a significant increase in p62/SQSTM1 levels, as compared to control RNAi transfection (FIG. 4A, right panel). Moreover, knock-down of ATG14L or Beclin 1 caused remarkably enhanced levels of LC3I or LC3II as compared to that resulted from the control RNAi treatment, suggesting the impairment in LC3I or LC3II turnover by autophagy (FIG. 4A).

In a parallel experiment, ATG14L protein levels were knocked down in MLE12 cells that stably expressed the autophagy reporter, GFP-LC3 fusion protein. In cells transfected with control RNAi, many small GFP-LC3 puncta were observed, reflecting the presence of basal level autophagosomes (FIG. 4B left panel). In contrast, ATG14L RNAi transfection resulted in the reduced number of small GFP-LC3 puncta, but the accumulation of many bright and large-size GFP-LC3 puncta (FIG. 4B, right panel). These GFP-LC3 puncta were labeled positively with p62/SQSTM1 (FIG. 4C) and ubiquitin (FIG. 4D), indicating that these puncta are ubiquitinated protein inclusions as previously shown in Atg5 or Atg7 KO tissues (Hara T. et al., Nature (2006); 441, 885; Komatsu M. et al., Nature (2006); 441, 880). These results collectively suggest that reduced expression of ATG14L leads to impaired autophagy. In addition, the increased levels of LC3II caused by knock-down of ATG14L or Beclin 1 suggests that function of Beclin 1 or ATG14L in autophagy are not through affecting directly the production of lipid-conjugated LC3II, consistent with the previous observation that deletion of Atg6 resulted in enhanced levels of lipidated form of Atg8 in yeast (Suzuki K. et al., Yeast (2004); 21, 1057-65).

Since Vps34/PtdIns3K plays a critical role in controlling autophagic activity (Backer J. M. Biochem J (2008); 410, 1-17) and ATG14L is a component of Beclin 1-Vps34/PtdIns3K complex, whether ATG14L can modulate Vps34/PtdIns3K activity was studied. A method which was previously shown in the analysis of UVRAG-Vps34/PtdIns3K function in vitro (Liang C. et al., Nat Cell Biol (2006); 8, 688-99) was adopted, but with modification by adding Vps15/p150 in the assay (Baker 3). Thus, HEK 293T cells with Myc-Vps34/PtdIns3K-Vps15/p150 and FLAG-ATG14L, either with or without Beclin 1-EGFP expression were co-transfected. The anti-myc immunoprecipitated Vps34/PtdIns3K protein was assayed for its lipid kinase activity by monitoring the incorporation of radioactive-³²P-ATP into PtdIns using thin layer chromatography (TLC) (FIG. 4E). The result showed that co-expression of ATG14L with Vps34/PtdIns3K-Vps15/p150 resulted in a 2.5 fold increase in lipid kinase activity of Vps34/PtdIns3K over that from co-expression of control FLAG plasmid and Vps34/PtdIns3K-Vps15/p150 (FIG. 4E). Interestingly, this ATG14L-mediated stimulation of Vps34/PtdIns3K activity occurred only when co-expressing Beclin 1. Together, the data suggest that over-expression of ATG14L enhances Vps34/PtdIns3K activity, and this specific effect depends on the presence of Beclin 1 in the Vps34/PtdIns3K complex.

Over-Expression of Beclin 1 and Atg14L Synergistically Causes Formation of Double-Membrane Structures that are Also Positive for Atg5 and Atg12

It was found that Beclin 1-EGFP stably expressed in HEK-293 cells was primarily diffusely distributed in cytoplasm (FIG. 5A, left panel), which is consistent with the previous report in transgenic mouse tissues (Arsov I. et al., Cell Death Differ (2008); 15, 1385-1395). A similar diffuse pattern of ATG14L-EGFP localization was observed in stably transfected HEK-293 cells (FIG. 5A, right panel). To further test the Beclin 1-ATG14L interaction in autophagy, ATG14L-EGFP and Beclin 1-AsRed were co-expressed in HeLa cells. The co-expression of ATG14L-EGFP and Beclin 1-AsRed (but not each alone) resulted in formation of numerous fluorescent puncta that were labeled with both ATG14L-EGFP (green) and Beclin 1-AsRed (red) (FIG. 5B). To further investigate the nature of these puncta, ultrastructural analysis was performed by electron microscopy. In HEK-293T cells co-expressing ATG14L-EGFP and Beclin 1-AsRed, a number of large-size structures (˜3-5 micron in diameter) (FIG. 5C1-2), which were virtually absent in single ATG14L-EGFP- or Beclin 1-AsRed-transfected cells were observed. Some of these structures displayed concentric “rings” with double membranes (FIG. 5C1); many are large vacuoles filled with materials of high electron density (FIG. 5C2). Interestingly, these large organelles were enwrapped with double-membranes (e.g., FIG. 5C2 inset), which are distinguished from typical aggresomes or protein aggregates (usually not associated with limiting membranes). Increased number of autophagosomes in the cells with overexpression of ATG14L-EGFP and Beclin 1-AsRed was also found (FIG. 5C3). In addition, as shown by anti-GFP staining, these large organelles were mostly positive for ATG14L-EGFP (FIG. 5C4).

To study the relationship of Beclin 1-ATG14L-labeled structures to other autophagy proteins, HeLa cells stably expressing GFP-LC3 with Beclin 1-Myc and ATG14L-AsRed plasmids were transfected. Again, co-expression of Beclin 1-Myc (blue) and ATG14L-AsRed (red) caused formation of Beclin 1-ATG14L puncta, and these puncta co-localized with GFP-LC3 (green) (FIG. 5D), suggesting that the Beclin 1-ATG14L structures likely recruit LC3. Moreover, when HeLa cells were co-transfected with ATG14L-AsRed, Beclin 1-Myc and EGFP-Atg12 (or EGFP-Atg5), it was found that ATG14L-Beclin 1 puncta (red and blue, respectively) co-localized with EGFP-Atg12 or EGFP-Atg5 (green) (FIG. 5E-F), suggesting that these Beclin 1-ATG14L structures may also recruit Atg12 and Atg5, whose conjugate is required for autophagosome expansion.

Taken together, the studies suggest that Beclin 1 and ATG14L synergistically promote the formation of double membranes during the process of autophagosome biogenesis. Furthermore, these studies also suggest a link of between Beclin 1-ATG14L interaction and the function of Atg5-Atg12 conjugate in autophagosome formation.

Rubicon is a Negative Regulator of Autophagy

To investigate the role of Rubicon in autophagy, RNAi knock down of endogenous Rubicon protein was conducted. Transfection of NIH 3T3 cells with specific Rubicon RNAi resulted in reduction of Rubicon protein levels as detected with anti-Rubicon antibody (FIG. 6A). In contrast to the result of ATG14L or Beclin 1 RNAi knock-down, Rubicon RNAi knock-down did not cause an increase in the levels of LC3II or p62/SQSTM1; rather, as compared to the control RNAi, Rubicon RNAi knock-down decreased the steady-state levels of LC3II and p62/SQSTM1, under both normal and nutrient starvation conditions (FIG. 6A). The reduced levels of p62/SQSTM1 suggest an up-regulated autophagic degradation; the reduced LC3I/II levels are consistent with a fast turnover of LC3I/II proteins through up-regulated autophagy. To examine the effect of Rubicon over-expression on autophagy, autophagic activity in either 293 cells stably transfected with Rubicon-EGFP (FIG. 6B, upper panel) or 293T cells transiently transfected with Rubicon-EGFP (FIG. 6B, lower panel) was analyzed. As shown by Western blot analysis with anti-p62/SQSTM1 antibody, the p62/SQSTM1 protein levels were markedly enhanced in cells either stably or transiently transfected with Rubicon-EGFP, as compared to those in the control cells transfected with EGFP (FIG. 6B). This result suggests that excess expression of Rubicon in cells inhibits autophagy.

As Rubicon is also associated with Beclin 1-Vps34/PtdIns3K complex, the possible effect of Rubicon on the lipid kinase activity of Vps34/PtdIns3K was also examined. Myc-Vps34/PtdIns3K-Vps15/p150 and FLAG-Rubicon in HEK 293T cells, either with or without Beclin 1-EGFP over-expression were co-expressed by using the same method as described previously (FIG. 4E). The lipid kinase assay as shown by TLC indicated that co-expression of Rubicon with Vps34/PtdIns3K resulted in a significant reduction in the lipid kinase activity of Vps34/PtdIns3K, as compared to that derived from co-expression of control FLAG plasmid and Vps34/PtdIns3K (FIG. 6C). Noticeably, the Rubicon-triggered decrease in Vps34/PtdIns3K activity occurred only in the absence, but not in the presence, of Beclin 1-EGFP over-expression (FIG. 6C). Therefore, in contrast to ATG14L, over-expressed Rubicon in cells inhibits Vps34/PtdIns3K lipid kinase activity, and this specific effect does not require Beclin 1.

To further investigate the mechanism by which over-expression of Rubicon blocks autophagy, a previously developed double-tagged LC3 reporter, mCherry-GFP-LC3, was used to examine whether Rubicon would affect autophagosome maturation step, such as autophagosome acidification following fusion with late endosomes/lysosomes (Pankiv S. et al., J. Biol. Chem. (2007); 282, 24131-24145). Since GFP, but not mCherry, is acid-labile, mature autophagosomes (at acidic pH) which are labeled with mCherry-GFP-LC3 will only emit red mCherry fluorescence; immature autophagosomes (at neural pH)-associated mCherry-GFP-LC3 will emit both red and green fluorescence and thus appear yellow when merging two colors. It was found that, in the co-transfected cells with FLAG-Rubicon and mCherry-GFP-LC3 plasmids, cells expressing both FLAG-Rubicon and mCherry-GFP-LC3 proteins contained primarily yellow fluorescent puncta (immature autophagosomes) (FIG. 6D, lower panel, two lower arrows), whereas cells expressing only mCherry-GFP-LC3 (FIG. 6D, lower panel, two upper arrows) or cells co-transfected with control vector FLAG and mCherry-GFP-LC3 (FIG. 6D, upper panel) contained a significantly higher numbers of red fluorescent “dots” (mature autophagosomes) than cells expressing both FLAG-Rubicon and mCherry-GFP-LC3 (FIG. 6E). This result suggests that over-expression of Rubicon blocks autophagy through inhibiting autophagosome acidification or maturation, and further suggests that Rubicon is a negative regulator of autophagy.

Aberrant Expansion of Late Endosomes/Lysosomes Caused by Over-Expression of Rubicon

Rubicon subcellular localization and function in cells were investigated. In contrast to ATG14L-EGFP or Beclin 1-EGFP, forced expression of Rubicon-EGFP only in stably transfected cells resulted in a punctate distribution of Rubicon-EGFP (FIG. 7A). To rule out the possibility that EGFP tagging is artificially linked to puncta formation, FLAG-Rubicon was expressed in cells and similar punctate localization of FLAG-Rubicon was observed. Some of these Rubicon-EGFP puncta were large in size and exhibited “ring” shapes (FIG. 7A, arrows marked with asterisks). To characterize the Rubicon-EGFP puncta, Rubicon-EGFP-transfected HeLa cells were stained with a series of antibodies that recognize various cellular organelles. These results showed that while some Rubicon-EGFP puncta or “rings” were partially labeled with early endosomal marker EEA1 (FIG. 5I), these structures were primarily co-localized with Lamp1, a late endosomal/lysosomal marker (FIG. 7A). Interestingly, some of Rubicon-EGFP puncta were positively stained with an antibody against lysobisphosphatidic acid (LBPA) (FIG. 7B), an unusual eukaryotic lipid found only in multi-vesicular body (MVB) (Kobayashi T. et al., Nature (1998); 392, 193), suggesting that some of the Rubicon-EGFP structures may be related to MVB (Katzmann D. J. et al., Nat Rev Mol Cell Biol (2002); 3, 893).

To examine the ATG14L-EGFP-labeled organelles at ultrastructural levels, electron microscopy analysis was performed. These results revealed the formation of many abnormally large-size vacuoles (1-5 μm in diameter) in ATG14L-EGFP-transfected cells (FIG. 7C). In contrast, these large structures were rarely observed in EGFP, ATG14L-EGFP or Beclin 1-EGFP transfected cells. Some of these large vacuoles contained high electron density (FIG. 7C1-2, arrows marked with **), characteristic of late endosomes/lysosomes; some had relatively less content with overall low electron density (more transparent), which may represent the early stage of endosomes (but with enlarged size) (FIG. 7C3, black arrows); interestingly, some enclosed numerous small vesicles of multiple-layers (FIG. 7C3-4, arrows marked with ‡), while others resembled multi-vesicular endosomal vesicles or MVB (Katzmann D. J. et al., Nat Rev Mol Cell Biol (2002); 3, 893) (FIG. 7C1-2, arrows marked with ‡). To ensure that these large organelles under EM correspond to those observed as large fluorescent Rubicon puncta (FIG. 7A), immuno-microscopic analysis was performed by using anti-GFP antibody and extensive staining by the gold particles on the large vacuoles was observed (FIG. 7D). These gold particles were found to be largely associated with limiting membranes both inside and outside, and the gold labeling was mostly robust outside of membranes (FIG. 7D).

Taken together, the studies suggest that over-expressed Rubicon not only associates with late endosomal/lysosomal membrane network, but also causes aberrant expansion of the late endosomes/lysosomes.

Rubicon-Resided Structures are PtdIns3(P)-Enriched and are not Associated with Beclin 1

To understand the functional connection of Rubicon to the Beclin 1-Vps34/PtdIns3K complex, the Rubicon-associated organelles in a relationship with Vps34/PtdIns3K activity and Beclin 1 inside cells was investigated. By bioinformatic analysis of Rubicon sequence, it was found that C-terminal region of Rubicon (a.a. 837-890) contained several cysteine residues (cysteine-rich domain) which are conserved in FYVE domain, a well-characterized motif specific for PtdIns(3)P binding (FIG. 8A) (Stenmark H. et al., FEBS Letters (2002); 513, 77). However, the sequence in Rubicon did not contain the key consensus sequences of typical FYVE domain, i.e., WxxD, R[R/K]HHCR and RVC (FIG. 8A, bars marked with asterisks). The binding of Rubicon to PtdIns(3)P using PtdIns(3)P-conjugated sephorase beads were examined. The result showed that although PtIns(3)P-beads efficiently pulled down 2×FYVE-EGFP from transfected cell lysate, little Rubicon-EGFP protein was recovered from PtdIns (3)P-bead precipitates, despite the comparable protein amounts of Rubicon-EGFP and 2×FYVE-EGFP used for the binding assay. This result suggests that unlike FYVE domain, Rubicon protein may not directly bind to PtdIns(3)P. Interestingly, when co-expressed in HeLa cells with p40 (phox)-PX-EGFP, a reporter for localization of PtdIns(3)P, Rubicon-AsRed showed extensive co-localization with p40 (phox)-PX-EGFP (FIG. 8B, upper panels) (as compared to ATG14L-AsRed or Beclin 1-AsRed), suggesting that Rubicon-associated structures (mostly abnormal late endosomes/lysosomes, FIG. 7) are enriched in PtdIns(3)P. Moreover, this result also indicates that Rubicon is closely associated with Vps34/PtdIns3K kinase activity in those structures. Consistent with the difference between 2×FYVE and Rubicon in binding to PtdIns(3)P, treatment of cells co-expressing Rubicon-AsRed and p40 (phox)-PX-EGFP with Wortmanin, an inhibitor of Vps34/PtdIns3K kinase, effectively dispersed p40(phox)-PX-EGFP puncta, but not the Rubicon-AsRed structures (FIG. 8B, lower panels). This result suggests that formation of Rubicon-associated structures does not rely on the presence of PtdIns(3)P.

To determine the specific sequence in Rubicon that is required for the association with the distinguished large structures, HeLa cells were transfected with individual plasmids encoding different truncated mutants of Rubicon. Confocal imaging revealed that Rubicon mutant truncated at the N-terminal RUN domain (Rubicon(ΔRUN)) still maintained similar puncta distribution as full-length Rubicon; whereas truncated mutant either lacking the C-terminal cysteine-rich domain (Rubicon(ΔC)) or both RUN and cysteine-rich domains (Rubicon(ΔRUNΔC)) became diffuse (FIG. 8D). This result suggests that the cysteine-rich domain of Rubicon is required for the formation of specific large structures which are associated with aberrant late endosomes/lysosomes and are enriched in PtdIns(3)P.

Finally, to determine whether Beclin 1 is involved in the formation of Rubicon-associated structures, Beclin 1-AsRed or FLAG-Beclin 1-CE mutant (containing the domains important for binding to Rubicon, FIG. 2) was expressed in cells stably transfected with Rubicon-EGFP protein. The localization of Beclin 1-AsRed (red) or FLAG-Beclin 1-CE (as shown by the staining with anti-FLAG antibody, red) was exclusive from Rubicon-EGFP-labeled puncta (green, FIG. 8D-E), suggesting that Beclin 1 is not present at Rubicon-EGFP-associated structures. To further examine whether formation of Rubicon-labeled structures does not require Beclin 1, Beclin 1 protein was knocked down by specific Beclin 1 RNAi in HEK 293 cells stably transfected with Rubicon-EGFP. Despite the significantly reduced expression of Beclin 1, the formation of Rubicon-EGFP puncta was barely affected (FIG. 8F). Therefore, these observations provide evidence that the association of Rubicon to late endosomal-lysosomal membrane network is independent of Beclin 1.

Taken together, the studies suggest an important role for the C-terminal cysteine-rich domain of Rubicon in the association of Rubicon with the late endosomes/lysosomes, which also recruits Vps34/PtdIns3K kinase activity. This action of Rubicon-Vps34/PtdIns3K (excluding Beclin 1), while over-expressed, may contribute to aberrant expansion of the late endosomes/lysosomes and deregulation of autophagy through interfering with autophagosome maturation process.

### 6.1.1 Discussion

In this study, the identification and characterization of Beclin 1 protein complexes that contain Vps34/PtdIns3K, a lipid kinase linked to several major vesicle trafficking pathways including endocytosis and autophagy in mammals (Odorizzi G. et al., Trends in Biochemical Sciences (2000); 25, 229; Futter, C. E. et al., J Cell Biol (2001); 155, 1251-64; Lindmo K. and Stenmark H., J Cell Sci (2006); 119, 605-614) were reported. The Beclin 1 protein complexes are uncovered through a unique mouse model, which provides an opportunity to capture Beclin 1-binding proteins under physiological condition. The studies have shown that Beclin 1, Vps34/PtdIns3K, p150 and UVRAG are the primary components of the Beclin 1 complex, and they likely exist in closely stoichiometric fashion; whereas two novel components of this complex, ATG14L and Rubicon, are found in much reduced amount. These observations immediately suggest that Beclin 1, p150, Vps34/PtdIns3K and UVRAG constitute the core of the Beclin 1 complex. Our results further suggest the existence of multiple Beclin 1-related protein complexes in vivo: a large Beclin 1 complex consisting of the six components Beclin 1, p150, Vps34/PtdIns3K, UVRAG, ATG14L and Rubicon; a smaller complex containing ATG14L-Beclin 1 but excluding Rubicon. In addition, Rubicon can be associated with Vps34/PtdIns3K in the absence of Beclin 1 or ATG14L (FIG. 8G). Interestingly, the studies did not detect other previously identified Beclin 1-associated proteins, such as nPIST (Yue Z. et al. Neuron (2002); 35, 921), Bcl-2 (Liang X. H. et al., J. Virol. (1998); 72, 8586-8596), Ambra-1 (Fimia G. M. et al., Nature (2007); 447, 1121-5) or Bif1 (Takahashi Y. et al., Nat Cell Biol (2007); 9, 1142-51). The absence of these proteins in the assay may suggest that their bindings to Beclin 1 is relatively unstable, transient, or occur only under specific conditions.

The mechanism underlying the regulation of autophagy by the Beclin 1-Vps34/PtdIns3K complex has not been shown previously. In this study, identification of the two components ATG14L and Rubicon of the Beclin 1-Vps34/PtdIns3K complex have begun to provide insights into the mechanism by which the Beclin 1-Vps34/PtdIns3K complex is connected to the control of autophagic activity. These results have shown that ATG14L and Rubicon are both involved in regulation of autophagy, but in opposing directions: while ATG14L positively regulates autophagy, Rubicon negatively regulates autophagy. These results have also shown that while ATG14L is able to stimulate Vps34/PtdIns3K kinase activity, Rubicon down-regulates Vps34/PtdIns3K kinase activity. Given the essential role of Vps34/PtdIns3K in controlling autophagic activity, ATG14L and Rubicon may regulate autophagy by binding to and thus directly modulating Vps34/PtdIns3K activity.

The study also reveals that ATG14L and Rubicon regulate autophagy by targeting distinct steps of the autophagic process. First, the results have collectively suggested that the functions of ATG14L and Beclin 1 are tightly connected. For example, in the study, ATG14L and Beclin 1 showed similar subcellular localization and behaved similarly in regulating Vps34/PtdIns3K activity and autophagy. Notably, when Beclin 1 protein levels were knocked down, ATG14L levels also diminished (FIG. 4A), suggesting that ATG14L-Beclin 1 complex may stabilize ATG14L protein and prevent its degradation. The synergistic effect of Beclin 1 and ATG14L in promoting formation of double-membraned, Atg5- and Atg12-positive vacuoles provides evidence for the function of ATG14L in early autophagosome biogenesis. In fact, local sequence homology between yeast Atg14 and ATG14L was also observed (FIG. 12). Thus the tightly connected function of Beclin 1 and ATG14L shown in this study may implicate ATG14L as the mammalian homologue of yeast Atg14 (Obara K. et al., Mol. Biol. Cell (2006); 17, 1527-1539). The specific function of Atg6/Atg14 in yeast autophagy (as opposed to CVT) may be conserved in Beclin 1/ATG14L for mammalian autophagy.

Second, although Rubicon can bind to Beclin 1-complex, over-expression of Rubicon was associated with the formation of abnormal late endosomes/lysosomes that is in the absence of Beclin 1. This result has suggested that certain function of Rubicon is independent of Beclin 1. However, the specific function of Rubicon may be associated with Vps34/PtdIns3K, as Rubicon-associated structures are extensively co-localized with PtdIns(3)P. In agreement with this notion, the study has shown that down-regulation of Vps34/PtdIns3K activity by Rubicon assayed in vitro occurs only in the absence of over-expressed Beclin 1. This result has raised a possibility that Rubicon-Beclin 1 binding may neutralize the negative effect of Rubicon on Vps34/PtdIns3K activity, thus implicating a role of Beclin 1 in autophagy regulation by specifically “checking” Rubicon function.

The observation that Rubicon was associated with endosomes (primarily late stage) has suggested that Rubicon-Vps34/PtdIns3K may be involved in regulation of endocytosis. To test it, whether over-expression of Rubicon affects endocytic internalization of a fluid phase marker (Horseradish peroxidase, HRP) was investigated. The result has suggested that over-expression of Rubicon does not significantly alter internalized HRP levels. In contrast, over-expression of Rubicon remarkably impaired the acidification of LC3-associated vacuoles, a step important for autophagosome degradation. This result has suggested that overactive Rubicon inhibits autophagosome maturation/degradation. The data have indicated an important role of Rubicon in autophagy control.

In summary, the study has discovered important physiological components in Beclin 1-Vps34/PtdIns3K protein complexes, and suggested the existence of multiple Beclin 1 protein complexes which are engaged in distinct functions in autophagy regulation (FIG. 8G). The dynamic change in protein composition between different functional complexes may play a central role in mediating Vps34/PtdIns3K activity that governs multiple events in autophagy.

### 6.1.3 Materials and Methods

Reagents and Antibodies

Dynabeads M-270 epoxy, NuPAGE Bis-Tris gels, Western blot transfer buffer, MES SDS running buffer, antioxidant, Lipofectamine 2000 kit and Lipofectamine RNAiMAX were purchased from Invitrogen (Carlsbad, Calif.). Modified Trypsin, EDTA-free protease inhibitor cocktail tablets, and FuGene 6 transfection reagent were purchased from Roche Diagnostics (Indianapolis, Ind.). Immobilon-P PVDF membrane was purchased from Millipore (Billerica, Mass.). GelCode Blue Stain Reagent, Trifluoroacetic acid, Tris(2-carboxyethyl)-phosphine hydrochloride, and the Micro BCA Protein Assay Reagent Kit were purchased from Pierce (Rockford, Ill.). Gel filtration calibrants were purchased from BioRad (Hercules, Calif.). Phosphatidylinositol was purchased from Avanti Polar Lipids (Alabaster, Ala.). Radio-active ³²P-ATP was purchased from PerkinElmer (Waltham, Mass.). G418 and anti-Myc affinity gel beads were purchased from Sigma (St Louis, Mo.). Beclin 1 siRNA was purchased from Dharmacon (Lafayette, Colo.); ATG14L siRNA was purchased from Invitrogen (Carlsbad, Calif.); and Rubicon siRNA and non-targeting siRNA control were purchased from Ambion (Austin, Tex.). Coated silica gel was purchased from EMD (Gibbstown, N.J.). Fluorescence mounting medium was purchased from Abeam (Cambridge, Mass.). Vectastain Elite ABC kit was purchased from Vector Laboratories (Burlingame, Calif.).

Polyclonal GFP antibody was raised against GST-tagged GFP and affinity purified (Cristea I. M. et al., Mol Cell Proteomics (2005); 4, 1933-1941). ATG14L and Rubiconpolyclonal antibodies were produced by injection of recombinant proteins in rabbits, as described in a separated section. Commercial antibodies used in this study include rabbit polyclonal LC3 antibody (1:1000, MBL, Woburn, Mass.), rabbit polyclonal antibody raised against the C-terminal of UVRAG (1:500, Abgent, San Diego, Calif.), rabbit polyclonal Beclin 1 antibody (1:600; Santa Cruz Biotechnology, Santa Cruz, Calif.). guinea pig polyclonal p62/SQSTM1 antibody (1:1000, American Research Products, Inc., Belmont, Mass.), mouse monoclonal Lamp 1 antibody (1:10, Developmental Studies Hybridoma Bank at the University of Iowa, Iowa City, Iowa), mouse monoclonal LBPA antibody (1:200, Echelon Biosciences Inc. Salt Lake City, Utah), rabbit polyclonal Vps34/PtdIns3K antibody (1:250, Zymed Laboratories, San Francisco, Calif.), mouse monoclonal c-myc antibody (1:1000; Santa Cruz Biotechnology, Inc., Santa Cruz, Calif.; Zymed Laboratories, San Francisco, Calif.), rabbit polyclonal mouse monoclonal M2 FLAG antibodies and mouse monoclonal β-actin antibodies (Sigma, St. Louis, Mo.), anti-rabbit Alexa Fluo 555 and Alexa 488 (1:1000, Invitrogen, Carlsbad, Calif.), Rhodomine Red conjugated secondary antibody (Jackson ImmunoResearch, West Grove, Pa.), anti-mouse and anti-rabbit Cy5 (1:500, Zymed Laboratories, San Francisco, Calif.), rabbit IgG (1:6000; Amersham, Pittsburgh, Pa.), mouse IgG (1:10000; Pierce, Rockford, Ill.).

Generation of Beclin 1-EGFP/+ BAC Transgenic Mouse

Beclin 1-EGFP mice were generated using BAC mouse transgenic techniques. A 455-base pair homology region immediately upstream of stop codon of beclin 1 gene (“A box”) was PCR amplified and inserted between AscI and SmaI sites of pLD53.SCA-E-B, and another 507-base pair homology region immediately downstream of stop codon was PCR amplified and inserted between PacI and StuI sites of the vector (“B box”) (Gong S. et al., Genome Res. (2002); 12, 1992-1998). Through homologous recombination, DNA sequence encoding EGFP protein followed by a stop codon was inserted into the BAC clone RP24-392C4 which contains all regulatory elements of beclin 1 gene to replace the original stop codon of beclin 1. The modified BAC clone was purified using CsCl gradient ultra-centrifugation, and used for pronuclear injection in FVB/NJ mice. All mice were backcrossed to C57BL/6J background for more than 10 generations before analysis.

Genotyping of Beclin 1-EGFP/+ Transgenic Mice

Genotype of mice from pronuclear injection was determined with either southern blot or PCR reaction. For Southern blot, a 0.5-1 inch piece of mouse tail was clipped from the tail end and incubated in lysis buffer (27% sucrose, 1×SSC, 1 mM EDTA, 1% SDS, 200 μg/ml proteinase K) at 55° C. overnight. After extractions with phenol:chloroform (1:1) and with chloroform, DNA was precipitated with ethanol and dissolved in TE buffer. Genomic DNA was digested with XhoI and XbaI, and was analyzed with southern blot using “A box” as the hybridization probe. For PCR reaction, the EGFP forward and reverse primer pairs are:

(SEQ ID NO: 13)
CCTACGGCGTGCAGTGCTTCAGC
and
(SEQ ID NO: 14)
CGGCGAGCTGCACGCTGCCGTCCTC.

Generation of Rescued Mice

Beclin 1-EGFP/+ transgenic mice were genetically crossed with beclin 1^{+/-} mice to generate rescued mice, in which both endogenous beclin 1 alleles were deleted and only beclin 1-EGFP transgene was expressed. First, beclin 1-EGFP/+ mice were crossed with beclin 1^{+/-} mice to obtain beclin 1^{+/-}; beclin 1-EGFP/+ mice. Then mice of this genotype were further crossed with beclin 1^{+/-} mice to obtain beclin 1^{-/-}; beclin 1-EGFP/+ mice. Tails were clipped from mice and protein was extracted in protein lysis buffer (20 mM HEPES, pH7.4/1 mM MgCl2/0.25 mM mM CaCl2/0.2% Triton X-100/150 mM NaCl/protease inhibitor and PMSF-pepstatin/DNaseI), after which Western blot with anti-Beclin 1 antibody was performed to confirm the deletion of endogenous Beclin1 protein. Multiple litters were produced and the numbers of mice of each genotype were counted to examine whether the Mendel's law was followed.

Affinity Purification and Mass Spectrometric Analysis

Affinity purification of Beclin 1 interacting proteins and mass spectrometric identification of these proteins were carried out as described in Wang et al. (2006), with slight modification. In brief, tissue extracts were obtained from both beclin 1^{+/-} and beclin 1^{-/-}; beclin 1-GFP/+ mice at 4 month of age by homogenizing 2 brains or 1 liver with a motor-driven homogenizer (speed 2.5, 12 strokes) in 4 ml buffer containing 0.32 M sucrose, 1 mM NaHCO₃, 20 mM HEPEs/pH 7.4, 1 mM MgCl₂, 0.25 mM CaCl₂, EDTA-free protease inhibitor cocktail, 200 μg/mL PMSF, pepstatin 4 μg/mL, and DNase I. The tissue extracts were centrifuged at 1,400 g for 10 minutes and the pellets were homogenized again in 4 ml buffer for 8 strokes and centrifuged again. Supernatants from the two homogenization steps were pooled, centrifuged at 750 g for 10 minutes. Aliquots of the resulting supernatants were used for gel filtration experiments. The remaining supernatants were diluted with equal volumes of 2× pull-out buffer and then 2 volumes of 1× pull-out buffer containing 20 mM HEPEs/pH 7.4, 1 mM MgCl₂, protease inhibitor cocktail, 100 μg/mL PMSF, 2 μg/mL pepstatin, 0.2% Triton X-100 and 150 mM NaCl. Each sample was incubated with 5 mg M-270 Epoxy Dyanbeads, which were pre-coated with affinity purified polyclonal anti-GFP antibody, for 1.5 hours at 4° C. Dynabeads were washed 5 times with 1× pull-out buffer and then incubated with 250 μl elution buffer containing 0.5 mM EDTA and 0.5 M NH₃.H₂O for 20 minutes at room temperature. The eluents were collected, frozen in liquid nitrogen and dried in a Speedvac. The purified complexes were resolved on a SDS-PAGE gel and stained with colloidal Coomassie. The entire gel lane of the beclin 1^{-/-}; beclin 1-GFP/+ sample was sliced into ˜30 2-mm wide pieces. Each gel piece was subject to in-gel digestion with trypsin and the resulting tryptic peptides were analyzed by a two-step mass spectrometric strategy to obtain MS and MS/MS spectra, using either in-house-constructed MALDI-QqTOF and MALDI-ion trap (LCQ DECA XP; Thermo Electron Corporation) mass spectrometers or ESI-LTQ mass spectrometer (Thermo Electron Corporation). Accurate masses of the tryptic peptides and the masses of their product ions were used to identify proteins in each gel piece using the computer search engines ProFound (http://prowl.rockefeller.edu/prowl-cgi/profound.exe), Xproteo (http://www.xproteo.com) and GPM (http://prowl.rockefeller.edu/tandem/thegpm_tandem.html) to search the most up-to-date NCBI non-redundant mouse protein database.

Plasmid Constructs

Total RNA was extracted from postnatal day 12 mouse whole brain using RNeasy mini kit (Qiagen, USA). Full length cDNAs of interest were synthesized with Omniscript RT kit (Qiagen, USA) and were used as templates for reverse transcription PCR amplifications with KOD HiFi DNA polymerase (Novagen, USA). Beclin1 was cloned into EcoRI and BamHI sites of pEGFP-N3 and pAs-Red vectors (Clonetech, USA). ATG14L was cloned into EcoRI and BamHI sites of pEGFP-N3, pAsRed and pCMV-FLAG (Sigma, St Louis, Mo.) vectors. Rubicon was cloned into HindIII and BamHI sites of pEGFP-N3, pAs-Red and pCMV-FLAG2 vectors. UVRAG was cloned into XhoI and BamHI sites of pEGFP-N3 vector. Single or combinations of Beclin 1 domains were cloned into EcoRI and BamHI sites of pCMV-FLAG2 vector. Truncated ATG14L mutants were cloned into EcoRI and BamHI sites of pEGFP-N3 vector. Truncated Rubicon mutants were cloned into KpnI and BamHI sites of pEGFP-N3 vector. EGFP-Atg12 and EGFP-Atg5 constructs were generously provided by Dr. X. Jiang. Myc-hVps34-hVps15-V5-His/pVITRO2 plasmid was a generous gift from Dr. J. Backer.

Generation of Antibodies

Full length cDNA of ATG14L was cloned into NdeI and BamHI sites of bacterial expression vector pET-28a(+) (Novagen, USA). Partial cDNA of Rubicon that corresponds to amino acids 220-941 was cloned into NheI and BamHI sites of pET-28a(+) vector. Recombinant 6×His-tagged ATG14L and Rubicon proteins were expressed in BL21-Codon-Plus (DE3)-RIL competent cells and purified with Ni-NTA agarose beads (Qiagen, USA). A total of 500 μg of purified recombinant protein was used for injections in rabbits to produce polyclonal antibodies (Cocalico Biologicals, Inc. Reamstown, Pa.). Sera from rabbits were either purified with protein-G column (GE Healthcare, Piscataway, N.J.) or further affinity purified with recombinant protein.

Cell Cultures and Transfections

Human embryonic kidney (HEK) 293 and 293T cells, Hela cells and NIH 3T3 cells were maintained in Dulbecco's modified Eagle's medium (DMEM) supplemented with 10% fetal bovine serum (Invitrogen, Carlsbad, Calif.). MLE12 cells were generously provided by Dr. C Münz and maintained in DMEM/F12 medium (ATCC, Manassas, Va.) supplemented with 0.005 mg/ml insulin, 0.01 mg/ml transferrin, 30 nM sodium selenite, 10 nM hydrocortisone, 10 nM beta-estradiol, 10 mM HEPES, 2 mM L-glutamine and 2% fetal bovine serum. Transient DNA transfection was performed with standard calcium phosphate precipitation procedure, FuGene 6 or Lipofectamine 2000 kit, following protocol provided by the manufacturers. siRNA transfection on NIH 3T3 cells were performed with Lipofectamine RNAiMAX kit following the reverse transfection protocol provided by the manufacturer. The sequences of siRNA are:

Beclin1, CAGUUUGGCACAAUCAAUA;
(SEQ ID NO: 11)
ATG14L, UUUGCGUUCAGUUUCCUCACUGCGC;
and
(SEQ ID NO: 12)
Rubicon, GCCUUCAGUCUAUGCCACA.

Generation of Stable Cell Lines

HEK 293 cells were transfected with pEGFP-N3 vector, Beclin 1-EGFP, ATG14L-EGFP or Rubicon-EGFP, and were selected with 800 μg/ml G418 for one week. Single colonies with EGFP expression were picked and culture was continued in medium supplemented with 200 μg/ml G418. Correct expression of EGFP-fusion protein was confirmed with Western blot using GFP antibody, after which cells were maintained in culture medium supplemented with 200 μg/ml G418.

In Vitro Protein Immunoprecipitation

DNA plasmids were transfected into HEK 293T cells for immunoprecipitation. For Co-immunoprecipitation experiments, two or three plasmids were transfected simultaneously in equal amount. Cells were lysed in immunoprecipitation lysis buffer (20 mM HEPES, pH7.4/1 mM MgCl2/0.25 mM CaCl2/0.2% Triton X-100/150 mM NaCl/protease inhibitor and PMSF-pepstatin/DNaseI). For immunoprecipitation with GFP, ATG14L or Rubicon antibody, Dynabeads M-270 E-proxy (Invitrogen, Carlsbad, Calif.) were conjugated with each antibody, and incubated with cell lysate at 4° C. for 2 hours. After the beads were washed in ATG14L or Rubicon lysis buffer for 5 times, proteins were eluted by incubating beads in elution buffer (0.5 mM EDTA, pH8/0.5N NH₃.H₂O) at room temperature for 20 minutes and dried with vacuum speed centrifuge. For FLAG-tagged protein IP, Anti-FLAG M2 affinity resin (Sigma, St Louis, Mo.) was used and the manufacture's protocol was followed.

Vps34/PtdIns3K Kinase Assay

Vps34/PtdIns3K kinase assay was performed as described (Miled N. et al., Science (2007); 317, 239-242). Myc-hVps34-hVps15-V5-His/pVITRO2 plasmid was transfected into HEK 293T cells in combination with other FLAG-tagged or EGFP-tagged plasmids. Cells were lysed in 1% Nonidet P-40 lysis buffer (20 mM Tris/pH 7.5, supplemented with 137 mM NaCl, 1 mM MgCl₂, 1 mM CaCl₂, 100 mM NaF, 10 mM sodium pyrophosphate, 100 μM Na₃VO₄, 10% glycerol, and 0.35 mg/ml phenylmethylsulfonyl fluoride/protease and phosphatase inhibitors). Immunoprecipitation was performed with anti-Myc affinity gel beads, following manufacture's protocol. Beads were washed in lysis buffer three times, followed by three washes in washing buffer (100 mM Tris-HCl/pH7.4 and 500 mM LiCl) and two washes in reaction buffer (10 mM Tri-HCl/pH7.4, 100 mM NaCl and 1 mM EDTA). Beads were resuspended in 60 μl of reaction buffer, followed by addition of 10 μl of 100 mM MnCl₂ and 10 μl of sonicated phosphatidylinositol (2 μg/μl). The reaction was started by the addition of 10 μl of 440 μM ATP containing 10 μCi of γ-³²P-ATP, and beads were incubated for 10 min at room temperature. μl 8 M HCl, and organic phase was?Reaction was terminated by adding 20 μl chloroform:methanol (1:1). Extracted phospholipids?extracted with 160 products were spotted on a coated silica gel and separated in chloroform:methanol:H₂O:ammonium hydroxide (9:7:1.7:0.3), followed by visualization with Typhoon 9400 Variable Imager (GE Healthcare Biosciences, Piscataway, N.J.).

Fluorescence Microscopy

Cultured cells were fixed in fresh 4% paraformaldehyde in 1×PBS, and permeabilized in PBS with either 0.05%-0.1% Triton X-100 or 0.01% saponin for 10-30 minutes at room temperature. After blocking in PBS containing 10% goat serum for 30 minutes to 1 hour at room temperature, cells were incubated in primary antibodies overnight at 4° C. After three washes in PBS, cells were incubated with fluorescent secondary antibodies for one hour at room temperature, followed by four washes in PBS. Cells were mounted with fluorescence mounting medium and examined under Zeiss (Göttingen, Germany) upright and invert confocal microscopes.

Electron Microscopy

For morphology EM, transfected HEK 293T cells were fixed in 2.5% glutaraldehyde (2.5GA) in 0.1M cacodylate buffer, pH 7.4 and processed by routine transmission electron microscopy procedure and embedded in Epon (Yue Z. et al., Neuron (2002); 35, 921).

For immuno-EM, transfected HEK 293T cells were fixed in fresh PLP fixative (4% paraformaldehyde, 0.01M periodate, 0.075M lysine, and 0.075M phosphate buffer/pH7.4) supplemented with 0.05% glutaraldehyde for one hour. After three washes in PBS, cells were permeabilized with 0.01% saponin in PBS supplemented with 0.1% BSA for 15 minutes at room temperature, and were incubated with primary antibody (anti-GFP) diluted in the same buffer overnight at 4° C. Vectastain Elite ABC kit was used for secondary antibody incubation, and cells were fixed again in 1.5% glutaraldehyde in 0.1 M cacodylate buffer supplemented with 5% sucrose. After three washes with 50 mM Tris/pH 7.4 supplemented with 7.5% sucrose, DAB reaction was performed with the same kit. The reaction was stopped with 50 mM Tris/pH 7.4 supplemented with 7.5% sucrose, and cells were processed for silver enhancement as described (Teclemariam-Mesbah R. et al., J. Histochem. Cytochem. (1997); 45, 619-622).

Blocks were cut with a diamond knife on a Leica UltracutE and ultra-thin (˜70 nm) sections were collected on uncoated 200-mesh grids and stained with Uranium and Lead. Grids were viewed with a TecnaiSpiritBT Transmissiiom Electron Microscope (FED at 80 KV and pictures were taken with Gatan 895 ULTRASCAN Digital Camera. Image levels were processed in Adobe Photoshop (Adobe Systems, San Jose, Calif.) to enhance contrast.

Gel Filtration

Liver and brain extracts from both beclin 1^{+/-} and beclin 1^{-/-}; beclin 1-GFP/+ mice at 4 month of age were prepared as described above. Cell extracts were prepared as previously described (ref, our J Neuro paper). Tissue and cell extracts were diluted with equal volumes of 2× pull-out buffer (see above) and incubated for 15 minutes at 4° C. The samples were then subject to ultracentrifugation at 100,000 g and the resulting supernatants were used for gel filtration experiments. Superdex 200 HR10/30 column (Pharmacia) was equilibrated with 2 bed volumes of filtered running buffer containing 20 mM HEPEs/pH 7.4, 1 mM MgCl₂, 500 μg/mL PMSF, 1 μg/mL pepstatin and 150 mM NaCl. The column was calibrated using Biorad gel filtration calibrant mixture, which are composed of thyroglobulin (670 kDa), γ-globulin (158 kDa), ovalbumin (44 kDa), myoglobin (17 kDa) and vitamin B₁₂ (1,350 Da). A spike of these calibrants (10 μl) was also added to each sample (240 μl) as internal calibrants. Both calibrants and samples were run at a flow rate of 0.2 ml/min. For each run, 2 bed volumes of buffer were used to elute the sample and a total of 80 fractions were collected 25-29 minutes after starting the runs and at a rate of 1 fraction/min. Two bed volumes of buffer were used to wash the column at the same flow rate in between two runs.

Statistical Analyses

Statistical analyses were carried out as described in Komatsu, M. et al. Proceedings of the National Academy of Sciences (2007); 104, 14489-14494.

### 6.2 Example 2

### The Effect of ATG14L and Rubicon to Breast Cancer

The ATG14L knock-out mice (ATG14L^{+/-} and ATG14L^{-/-}) were made. Genotyping of these Atg14L knock-out mice and embryos were performed (FIG. 18). These results showed that homozygous ATG14L^{-/-} was embryonic lethal (see the first lane in FIG. 18). The hertozygous ATG14L^{+/-} knock-out mice, although were not observed to be embryonic lethal, were expected to develop tumors. Then Beclin 1 and ATG14L were detected in various breast cancer cell lines: MD231, MD361, MD453, T47D, MCF-7, HBL100, ZR75.1 (FIG. 19). Beclin 1 and ATG14L had lower or no expressions in some of these breast cancer cell lines. Compared to its expression in other breast cancer cell lines, Beclin 1 had lower expression in HBL100 cell lines, and no expression in MD231 cell lines. Compared to its expression in other breast cancer cell lines, ATG14L had greatly lower expression in MD361 cell line, and no expression in T47D and MCF-7 cell lines. These results suggest the potential role of ATG14L to breast cancer treatment, for example, an agent that increases the expression of ATG14L can be used to treat breast cancer. This discovery suggests an agent that increases the expression of ATG14L and/or an agent that decreases the expression of Rubicon, may be used for treating breast cancer.

In addition, the effect of RNAi knock-down of Beclin 1, ATG14L and Rubicon on cell number of 3T3 cell lines was studied (FIG. 20A). The results have shown that the knock-down of Beclin 1 and ATG14L caused over-proliferation of 3T3 cells, however, the knock-down of Rubicon slowed down the cell proliferation or growth in 3T3 cells. Furthermore, under nutrient-rich conditions, ATG14L siRNA treatment caused a slight decrease in the rate of degradation of long-lived proteins (˜10%, P=0.007) when compared with control siRNA treatment. However, this rate was markedly reduced upon nutrient withdrawal (˜37%, P=5×10-6); this effect of ATG14L siRNA was diminished in the presence of 3-methyladenine, an inhibitor of autophagy (FIG. 20B).

### 6.3 Example 3

TAU is a protein implicated in Alzheimer's disease. Accumulation of phosphorylation of TAU has been seen in Alzheimer's disease. First, the effect of Beclin 1 deficiency on phosphorylation levels of TAU was examined. The results have shown that Beclin 1 deficiency increased total TAU and Tau phosphorylation levels (FIG. 21). Beclin 1^{+/-} mice enhanced total and hyperphosphorylated Tau levels. For example, Beclin 1^{+/-} mice showed increased Tau phosphorylation and total Tau levels at one year, specifically at T231 (FIG. 21A-B). The result has also shown that the enhanced phosphorylation in Beclin 1^{+/-} mice was age dependent, since there was no clear difference between the Beclin 1^{+/-} mice at one year and Beclin 1^{+/+} mice at six months (FIG. 21C). However, EMX-Cre Beclin F/F mice at p25 (EXM-Cre conditional knockout) mice only showed enhanced phosphorylation of Tau at T231 and 5235 in comparison to wildtype littermate control. These results have shown that Beclin 1 deficiency was associated with increased accumulation of Tau phosphorylation which is observed in Alzheimer's disease.

Various publications are cited herein, the contents of which are hereby incorporated by reference in their entireties.

SEQUENCES

SEQ ID NO: 1: amino acid sequence of murine ATG14L protein (492 amino acids)
MASPSGKGSWTPEAPGFGPRALARDLVDSVDDAEGLYVAVERCPLCNTTRRRLTCAKC
VQSGDFVYFDGRDRERFIDKKERLSQLKNKQEEFQKEVLKAMEGKRLTDQLRWKIMSC
KMRIEQLKQTICKGNEEMKKNSEGLLKNKEKNQKLYSRAQRHQEKKEKIQRHNRKLGD
LVEKKTIDLKSHYERLARLRRSHILELTSIIFPIDEVKTSGRDPADVSSETDSAMTSSMVSK
LAEARRTTYLSGRWVCDDHNGDTSISITGPWISLPNNGDYSAYYNWVEEKKTTQGPDM
EHNNPAYTISAALGYATQLVNIVSHILDINLPKKLCNSEFCGENLSKQKLTRAVRKLNAN
ILYLCSSQHVNLDQLQPLHTLRNLMHLVSPRSEHLGRSGPFEVRADLEESMEFVDPGVA
GESDASGDERVSDEETDLGTDWENLPSPRFCDIPSQPVEVSQSQSTQVSPPIASSSAGGMI
SSAAASVTSWFKAYTGHR
SEQ ID NO: 2: amino acid sequence of murine Rubicon protein (941 amino acids)
MRPEGAGMDLGGGDGERLLEKSRREHWQLLGNLKTTVEGLVSANCPNVWSKYGGLER
LCRDMQNILYHGLIHDQVCCRQADYWQFVKDIRWLSPHSALHVEKFISLHESDQSDTDS
VSERAVAELWLQHSLQCHCLSAQLRPLLGDRQYIRKFYTETAFLLSDAHVTAMLQCLE
AVEQNNPRLLAQIDASMFARKQESPLLVTKSQSLTALPGSTYTPPASYAQHSYFGSSSSL
QSMPQSSHSSERRSTSFSLSGPSWQPQEDRECLSPAETQTTPAPLPSDSTLAQDSPLTAQE
MSDSTLTSPLEASWVSSQNDSPSDVSEGPEYLAIGNPAPHGRTASCESHSSNGESSSSHLF
SSSSSQKLESAASSLGDQEEGRQSQAGSVLRRSSFSEGQTAPVASGTKKSHIRSHSDTNIA
SRGAAEGGQYLCSGEGMFRRPSEGQSLISYLSEQDFGSCADLEKENAHFSISESLIAAIEL
MKCNMMSQCLEEEEVEEEDSDREIQELKQKIRLRRQQIRTKNLLPAYRETENGSFRVTSS
SSQFSSRDSTQLSESGSAEDADDLEIQDADIRRSAVSNGKSSFSQNLSHCFLHSTSAEAVA
MGLLKQFEGMQLPAASELEWLVPEHDAPQKLLPIPDSLPISPDDGQHADIYKLRIRVRGN
LEWAPPRPQIIFNVHPAPTRKIAVAKQNYRCAGCGIRTDPDYIKRLRYCEYLGKYFCQCC
HENAQMVVPSRILRKWDFSKYYVSNFSKDLLLKIWNDPLFNVQDINSALYRKVKLLNQ
VRLLRVQLYHMKNMFKTCRLAKELLDSFDVVPGHLTEDLHLYSLSDLTATKKGELGPR
LAELTRAGAAHVERCMLCQAKGFICEFCQNEEDVIFPFELHKCRTCEECKACYHKTCFK
SGRCPRCERLQARRELLAKQSLESYLSDYEEEPTEALALEATVLETT
SEQ ID NO: 3: amino acid sequence of human ATG14L protein (492 amino acids)
MASPSGKGARALEAPGCGPRPLARDLVDSVDDAEGLYVAVERCPLCNTTRRRLTCAKC
VQSGDFVYFDGRDRERFIDKKERLSRLKSKQEEFQKEVLKAMEGKWITDQLRWKIMSC
KMRIEQLKQTICKGNEEMEKNSEGLLKTKEKNQKLYSRAQRHQEKKEKIQRHNRKLGD
LVEKKTIDLRSHYERLANLRRSHILELTSVIFPIEEVKTGVRDPADVSSESDSAMTSSTVS
KLAEARRTTYLSGRWVCDDHNGDTSISITGPWISLPNNGDYSAYYSWVEEKICTTQGPD
MEQSNPAYTISAALCYATQLVNILSHILDVNLPKKLCNSEFCGENLSKQKFTRAVKKLN
ANILYLCFSQHVNLDQLQPLHTLRNLMYLVSPSSEHLGRSGPFEVRADLEESMEFVDPG
VAGESDESGDERVSDEETDLGTDWENLPSPRFCDIPSQSVEVSQSQSTQASPPIASSSAGG
MISSAAASVTSWFKAYTGHR
SEQ ID NO: 4: amino acid sequence of human Rubicon protein (972 amino acids)
MRPEGAGMELGGGEERLPEESRREHWQLLGNLKTTVEGLVSTNSPNVWSKYGGLERLC
RDMQSILYHGLIRDQACRRQTDYWQFVKDIRWLSPHSALHVEKFISVHENDQSSADGAS
ERAVAELWLQHSLQYHCLSAQLRPLLGDRQYIRKFYTDAAFLLSDAHVTAMLQCLEAV
EQNNPRLLAQIDASMFARKHESPLLVTKSQSLTALPSSTYTPPNSYAQHSYFGSFSSLHQS
VPNNGSERRSTSFPLSGPPRKPQESRGHVSPAEDQTIQAPPVSVSALARDSPLTPNEMSSS
TLTSPIEASWVSSQNDSPGDASEGPEYLAIGNLDPRGRTASCQSHSSNAESSSSNLFSSSSS
QKPDSAASSLGDQEGGGESQLSSVLRRSSFSEGQTLTVTSGAKKSHIRSHSDTSIASRGAP
ESCNDKAKLRGPLPYSGQSSEVSTPSSLYMEYEGGRYLCSGEGMFRRPSEGQSLISYLSE
QDFGSCADLEKENAHFSISESLIAAIELMKCNMMSQCLEEEEVEEEDSDREIQELKQKIRL
RRQQIRTKNLLPMYQEAEHGSFRVTSSSSQFSSRDSAQLSDSGSADEVDEFEIQDADIRR
NTASSSKSFVSSQSFSHCFLHSTSAEAVAMGLLKQFEGMQLPAASELEWLVPEHDAPQK
LLPIPDSLPISPDDGQHADIYKLRIRVRGNLEWAPPRPQIIFNVHPAPTRKIAVAKQNYRC
AGCGIRTDPDYIKRLRYCEYLGKYFCQCCHENAQMAIPSRVLRKWDFSKYYVSNFSKD
LLIKIWNDPLFNVQDINSALYRKVKLLNQVRLLRVQLCHMKNMFKTCRLAKELLDSFDT
VPGHLTEDLHLYSLNDLTATRKGELGPRLAELTRAGATHVERCMLCQAKGFICEFCQNE
DDIIFPFELHKCRTCEECKACYHKACFKSGSCPRCERLQARREALARQSLESYLSDYEEE
PAEALALEAAVLEAT
SEQ ID NO: 5: nucleotide sequence of murine ATG14L gene (1479 nucleotides)
atggcgtctcccagtgggaagggatcttggacgcccgaggctcctggttttgggccgcgggcgctagcacgggacctggtggactcggt
ggacgacgccgagggcctttacgtggctgttgagcggtgtcctctgtgcaacaccactcgccggcggttgacttgcgccaagtgcgtccag
agcggtgatttcgtctatttcgacggccgcgaccgggagaggtttattgacaagaaggaaagactaagccaacttaagaacaagcaagaa
gaatttcagaaagaagtactaaaagctatggaaggaaagcggcttacagatcagttgagatggaaaataatgtcatgcaagatgaggattga
acagctgaagcaaacaatatgtaaaggaaatgaggaaatgaagaaaaattctgaaggtctcctcaagaacaaggaaaagaaccagaagct
ttacagccgagcacagcggcaccaagagaaaaaggagaagattcagcggcacaaccgcaagcttggggacctggtggagaagaagac
cattgacttgaagagtcactatgagcggttggcgcggcttcgaaggtcacacatcctagagctcacctccatcatattcccaatcgacgaagt
gaagacttctgggagagaccctgcagacgtgtcttcagagactgacagtgccatgacctcaagcatggtgagcaagcttgctgaggcccg
gaggacaacttacctctctggaagatgggtctgtgatgaccacaatggtgacaccagcattagcatcacaggcccgtggattagcctaccaa
acaacggggactactctgcttactacaattgggtagaagagaagaaaacaacccaaggacctgacatggagcataacaaccccgcctaca
ctatcagcgccgcgctgggctacgccacgcagctcgtcaacattgtgtctcacatacttgacatcaatcttcccaaaaagctgtgcaacagc
gagttctgtggcgaaaacctcagcaagcagaaactgacacgcgcagtgaggaaactgaacgcaaacatcctttacctttgttcttctcagcat
gtaaatctggatcagttgcaaccactgcacacactcaggaacctgatgcacttggtcagcccgcgctctgagcacctaggcaggtcaggac
cctttgaagttcgagcagacctcgaggagtccatggaatttgtggaccctggagttgctggagaatcagacgcgagtggagatgagcgtgt
aagcgatgaggagactgacctgggcacagactgggagaacctgccaagcccccgattctgtgacatcccttcccagccggtggaagtgtc
ccagagccagagcacccaggtgtccccacccattgccagcagcagcgctggtgggatgatctcctccgctgcggcctcggtgacctcttg
gttcaaagcttacactggacaccgctaa
SEQ ID NO: 6: nucleotide sequence of human ATG14L gene (1479 nucleotides)
atggcgtctcccagtgggaagggagcccgggcgctggaggctcctggctgcgggccccggccgctcgcccgggacctggtggactcc
gtggacgatgcggaggggctgtacgtggctgtggagcgctgcccgctgtgcaacactacccgccggcggctgacctgcgccaaatgcgt
tcagagcggcgatttcgtctacttcgacggccgcgaccgggagaggtttatcgacaagaaggaaaggttaagccgacttaagagcaagca
agaagaatttcagaaagaagtgttaaaagctatggaaggaaaatggataacagatcagttgagatggaaaataatgtcctgcaagatgagg
attgaacagttaaaacaaacaatatgtaaaggaaatgaagaaatggagaaaaattctgaaggccttctcaaaaccaaggaaaagaatcaga
agctttacagtcgagcacaacggcaccaagagaaaaaggagaagattcagaggcataatcgcaaacttggtgacctggtagaaaaaaag
accattgacttaagaagtcattatgagcgtctggcaaatcttcgacgatcccatatattagagctcacctctgtcatttttccaatcgaggaagta
aagacgggtgtgagagaccccgcagatgtgtcttcagagagtgacagtgccatgacctccagcactgtgagcaagcttgctgaagcccgg
aggacaacttacctctcaggacgatgggtctgtgacgatcacaacggagacaccagcattagcattacagggccttggattagcctccctaa
caatggggactactctgcctactacagctgggtggaggagaagaaaacaacccaggggcctgacatggagcagagtaaccctgcctaca
ccatcagtgctgcgctgtgctatgcaactcagctggtcaacattctgtctcatatacttgatgtaaatcttcccaaaaagctctgcaacagtgaat
tttgtggcgaaaatctaagcaagcagaaatttactcgagcagtgaagaaactgaatgcaaatattctttacctttgtttttctcagcatgtaaattta
gatcaattacaaccactgcataccctcaggaatctaatgtacctggtcagtccaagctctgaacacctaggcaggtcagggccctttgaagta
cgagcagaccttgaggagtccatggaatttgtggatcccggagttgctggagaatcagatgagagcggagatgagcgcgtcagcgatgaa
gaaaccgacctgggcacagactgggagaacttgcctagtccccggttttgtgatatcccttcccagtctgtggaagtctcccagagtcagag
cacccaggcgtccccacccatcgcgagcagcagtgcaggtgggatgatctcctctgcagcagcctcggtgacctcctggtttaaagcttac
actggacaccgttaa
SEQ ID NO: 7: nucleotide sequence of murine Rubicon gene (2826 nucleotides)
atgcgtccggagggcgcgggaatggatcttggcggcggcgatggggagcgtctgctagagaagagcaggagggagcactggcagctg
ctgggtaatttgaagacgactgtggaaggtttggtgtcagccaactgccccaatgtctggtccaagtatggtggcctggagcggctgtgcag
ggacatgcagaacatcctgtatcatgggctcatccatgaccaggtgtgttgccgccaggctgattactggcagtttgtgaaagacattcggtg
gctcagcccgcactcagcccttcacgtggagaagttcatcagtttgcatgagagcgaccagagcgacactgacagcgtgagtgagcgtgc
tgttgcagagctgtggctgcagcatagcctgcagtgccactgcctctcagcccagctccggcctctgctcggggacagacagtacatcaga
aaattctacacagaaactgccttcctgctgagtgacgcccacgtcacagccatgctccagtgcctggaagcagtggaacagaacaaccccc
gtatctggctcagatcgatgcatccatgtttgccagaaagcaggagagcccgctgctggtcacaaagagccagagtctgaccgctctgcct
ggttccacatacacccctccagctagctatgctcagcattcctactttgggtcctcctccagccttcagtctatgccacagtccagccacagct
cagagagaagatctacttccttttcactctctggcccttcctggcaacctcaagaagacagagaatgcctctcaccagcagagactcaaacc
accccagctccgctgccttcagactctactctagcccaggattccccactgacagcacaggagatgagcgacagcactctgactagccccc
tagaggcatcctgggtcagcagccagaatgactccccaagtgatgtcagtgaggggcccgagtacctggccattggcaacccagcccctc
atggccggactgccagttgtgagagtcacagcagcaacggcgagagcagcagctctcacttgttctcctccagcagctctcaaaagctgga
gtctgctgcctcttctctaggggaccaagaggaaggcaggcagagtcaggcaggcagcgtccttcgcaggtccagcttctcagaagggca
gacagcccctgtcgccagcgggacaaagaagagccatattcgctcccactcggacaccaacattgcctccagaggagccgcagagggt
ggtcagtacctctgctcaggagaaggcatgtttagaagaccatcagaaggacagtccctcatcagttacctctctgagcaagactttggcag
ctgtgcagacctggaaaaggaaaacgcccacttcagcatctccgagtccttgattgccgccattgagctgatgaagtgcaacatgatgagcc
agtgtctagaagaggaggaagtagaggaggaagatagtgaccgagagatccaggaactgaagcagaagatccgccttcggcgccagca
gatccgcaccaaaaacctgctccctgcgtaccgggagactgagaatggaagcttccgggtcacctccagcagctcccagttcagttcacg
ggattcgacgcagctctctgagtccggctctgctgaggatgctgacgacttggaaatccaagacgctgacatccggaggagtgcagtctca
aacggcaaatcatccttctcccagaatctctcgcactgcttcctgcactccacatcggctgaggcagtggccatggggcttctgaagcagttt
gaggggatgcagcttccagctgcctcggagctcgagtggctagtcccagagcatgatgctccacagaagctcctacccatccccgactcc
ctgcccatctcaccagatgacgggcagcacgcggacatctacaagctgcgaatccgcgtccgtggcaacctggagtgggctccgccccg
gccgcagatcatttttaatgttcatccagcccccacgaggaagattgccgtggccaagcagaattaccgctgtgcgggatgtggcatccgga
cggaccccgactatatcaagcggctgcggtactgtgagtacctaggcaagtacttctgccagtgctgccacgagaacgcccagatggtcgt
ccccagccgcattctgcgcaaatgggacttcagcaagtactatgtcagcaacttctccaaggacctgctcctgaagatctggaatgatcctct
cttcaatgtgcaggacatcaatagcgcactctacaggaaggtcaagctgcttaaccaagtccggctgctgcgggttcagagtaccacatga
agaacatgtttaagacatgccgactggccaaagagctcctggattcctttgacgtggttccaggccacctgacagaggacctccatctgtact
cgctgagtgacctgactgcaaccaagaagggagaactggggccccggctggcggagctcactagagcgggagccgcccacgtggaga
gatgcatgctgtgtcaggccaagggcttcatctgtgaattctgtcagaatgaggaggatgtcatctttccttttgagctgcataagtgccggact
tgtgaagagtgtaaagcgtgttaccataaaacttgcttcaagtctggacgctgcccccggtgtgagcggctgcaggcccgacgggagctgc
tggccaagcagagcctggagtcctacctgtctgactatgaggaggagcccacagaagccttggctctagaggccaccgtcctagagacca
cctga
SEQ ID NO: 8: nucleotide sequence of human Rubicon gene (2919 nucleotides)
atgcggccggagggcgcgggaatggagctcggaggcggcgaggagcgcctgcctgaggagagcaggagggagcactggcagttgc
tgggtaatttgaagacgacggtggagggtttggtatcaaccaacagccccaacgtaggtctaagtatggtggcttggagcggatttgcagg
gacatgcagagcatcctctatcacgggcttatccgtgaccaggcgtgccgccgccagacggattactggcagttcgtgaaagacatccggt
ggctcagtccccactcagcccttcacgtggagaagttcatcagcgtgcacgagaacgaccagagcagtgctgatggtgccagtgaacgtg
ctgttgccgagctgtggctgcagcacagcctgcagtaccactgcctctcagcccagctccggcccctgctcggggatagacagtatatcag
aaaattctacacagatgctgccttcctgctaagtgacgctcatgtcacggccatgctgcagtgcctggaagcagtggaacagaacaacccc
cgcctcctggctcagatcgatgcgtccatgtttgccagaaagcacgagagcccgctcctggtgacaaagagccagagcctgacagccctg
cccagttccacatacacccctccaaacagctatgctcagcattcctactttgggtccttctctagcctccaccaatccgtgcccaacaatggct
cagagagaagatctacttcctttccactctctggccctccccggaaacctcaagaaagcagagggcacgtctcaccagcagaggatcaaac
catccaagcccccccagtttcagtctctgcactagccagggattcccctttgaccccaaatgaaatgagctccagtactctgaccagccccat
agaggcatcctgggtcagcagccagaatgattccccaggtgatgccagtgaggggcctgagtacctggccattggcaacttggacccccg
aggccggactgccagagtcagagtcacagcagcaatgccgagagcagcagttccaatttgttctcctccagcagacccagaagccaga
ttctgctgcctcttccttaggggaccaggaaggaggtggggagagccagctgtccagtgtcctccgcaggtccagcttctcagaggggca
gacactcactgtcaccagtggggcaaagaaaagccacattcgctcccattcggataccagcattgcctccaggggagctccagaatcctgc
aatgataaggcgaagttgagaggccctttgccctactctggtcaaagcagtgaagtcagcacacccagctctctgtacatggaatatgaagg
tggtcggtacctgtgctcaggggaaggcatgttccgaagaccatcagaaggacagtccctcatcagctacctctctgagcaagacttcggc
agctgtgccgacctggaaaaggagaatgcccacttcagcatctcagagtccttaattgctgccatcgagctaatgaagtgcaacatgatgag
ccagtgcctagaggaggaggaagtggaagaggaagacagtgatagagagatccaggagctgaagcagaagatccgccttcggcgcca
gcaaatccgcaccaagaacctgctccccatgtaccaggaggctgagcacggaagctttcgggtcacctccagcagctcccagttcagctc
acgtgattcggcacagctctctgactctggctctgctgatgaggttgatgaatttgaaatccaagatgctgacatcagaaggaacacagcctc
aagcagcaaatccttcgtttcctcccagtccttctcccactgcttcctgcactccacgtctgctgaggcggtggccatggggctcctgaagca
gtttgaggggatgcagattccagccgcctcggagctggagtggcttgtcccggagcatgatgcccctcagaagctcctgcccattcctgact
cactgcccatctcaccggatgacgggcagcacgctgacatctacaagctgcggattcgtgttcgtggcaacttggagtgggccccgcccc
ggcctcagataatttttaatgttcatccagccccaacgaggaaaattgccgtggccaagcagaattaccgctgtgcaggatgtggcatccgg
actgaccctgattacatcaagcgactgcggtactgtgagtacctgggcaagtacttctgccagtgctgccacgagaatgcccagatggccat
ccccagccgggttctgcgcaagtgggacttcagcaagtactacgtcagcaacttctccaaggacctgctcattaagatctggaatgatcctct
cttcaacgtgcaggacataaacagtgccctctataggaaggtcaagctgctcaatcaagtccggctgctgcgggtccagctgtgtcacatga
agaacatgttcaagacttgccgactggccaaggagcttctggattcctttgacacagtcccaggccacctgacagaggacctccacctgtac
tcactgaatgacctgactgcgaccaggaagggggagctggggccccggatgctgagctcaccagggcaggggctacccatgtggaga
gatgcatgactgccaagccaaaggcttcatctgtgagttctgtcagaatgaggatgacatcatctttccctttgagctccataagtgccggac
ctgtgaagagtgtaaagcgtgttaccataaagcctgcttcaagtctggaagctgtccgcgctgcgagcggctgcaggcccggcgggaggc
actggccaggcagagcctggagtcttacctgtcagactacgaggaggagcccgcggaagcgctggccctggaagccgccgtcctggag
gccacctga
SEQ ID NO: 9: amino acid sequence of human Beclin protein (450 amino acids)
MEGSKTSNNSTMQVSFVCQRCSQPLKLDTSFKILDRVTIQELTAPLLTTAQAKPGETQEE
ETNSGEEPFIETPRQDGVSRRFIPPARMMSTESANSFTLIGEASDGGTMENLSRRLKVTGD
LFDIMSGQTDVDHPLCEECTDTLLDQLDTQLNVTENECQNYKRCLEILEQMNEDDSEQL
QMELKELALEEERLIQELEDVEKNRKIVAENLEKVQAEAERLDQEEAQYQREYSEFKRQ
QLELDDELKSVENQMRYAQTQLDKLKKTNVFNATFHIWHSGQFGTINNFRLGRLPSVPV
EWNEINAAWGQTVLLLHALANKMGLKFQRYRLVPYGNHSYLESLTDKSKELPLYCSGG
LRFFWDNKFDHAMVAFLDCVQQFKEEVEKGETRFCLPYRMDVEKGKIEDTGGSGGSYS
IKTQFNSEEQWTKALKFMLTNLKWGLAWVSSQFYNK
SEQ ID NO: 10: amino acid sequence of murine Beclin protein (448 amino acids)
MEGSKASSSTMQVSFVCQRCSQPLKLDTSFKILDRVTIQELTAPLLTTAQAKPGETQEEE
ANSGEEPFIETRQDGVSRRFIPPARMMSTESANSFTLIGEASDGGTMENLSRRLKVTGDL
FDIMSGQTDVDHPLCEECTDTLLDQLDTQLNVTENECQNYKRCLEILEQMNEDDSEQLQ
RELKELALEEERLIQELEDVEKNRKVVAENLEKVQAEAERLDQEEAQYQREYSEFKRQQ
LELDDELKSVENQVRYAQIQLDKLKKTNVFNATFHIWHSGQFGTINNFRLGRLPSVPVE
WNEINAAWGQTVLLLHALANKMGLKFQRYRLVPYGNHSYLESLTDKSKELPLYCSGGL
RFFWDNKFDHAMVAFLDCVQQFKEEVEKGETRFCLPYRMDVEKGKIEDTGGSGGSYSI
KTQFNSEEQWTKALKFMLTNLKWGLAWVSSQFYNK
SEQ ID NO: 11: nucleotide sequence of siRNA targeted to ATG14L (25 nucleotides)
UUUGCGUUCAGUUUCCUCACUGCGC
SEQ ID NO: 12: nucleotide sequence of siRNA targeted to Rubicon (19 nucleotides)
GCCUUCAGUCUAUGCCACA
SEQ ID NO: 13: nucleotide sequence of EGFP forward primer for PCR reaction (23 nucleotides)
CCTACGGCGTGCAGTGCTTCAGC
SEQ ID NO: 14: nucleotide sequence of EGFP reverse primer for PCR reaction (25 nucleotides)
CGGCGAGCTGCACGCTGCCGTCCTC

What is claimed is:

1. An isolated polypeptide, comprising an amino acid sequence selected from the group consisting of:
a) an amino acid sequence that is up to 99% identical to SEQ ID NO: 1, comprising at least one sequence selected from the group consisting of amino acids 75 to 95 of SEQ ID NO: 1 and amino acids 148 to 178 of SEQ ID NO: 1;
b) an amino acid sequence that is up to 99% identical to SEQ ID NO: 2, comprising at least the sequence of amino acids 488 to 508 of SEQ ID NO: 2;
c) an amino acid sequence that is up to 99% identical to SEQ ID NO: 3, comprising at least one sequence selected from the group consisting of amino acids 75 to 95 of SEQ ID NO: 3 and amino acids 148 to 178 of SEQ ID NO: 3; and
d) an amino acid sequence that is up to 99% identical to SEQ ID NO: 4, comprising at least the sequence of amino acids 518 to 538 of SEQ ID NO: 4.

2. The isolated polypeptide of claim 1, which comprises an amino acid sequence that is up to 99% identical to SEQ ID NO: 1, comprising at least one sequence selected from the group consisting of amino acids 75 to 95 of SEQ ID NO: 1 and amino acids 148 to 178 of SEQ ID NO: 1.

3. The isolated polypeptide of claim 1, which comprises an amino acid sequence that is up to 99% identical to SEQ ID NO: 2, comprising at least the sequence of amino acids 488 to 508 of SEQ ID NO: 2.

4. The isolated polypeptide of claim 1, which comprises an amino acid sequence that is up to 99% identical to SEQ ID NO: 3, comprising at least one sequence selected from the group consisting of amino acids 75 to 95 of SEQ ID NO: 3 and amino acids 148 to 178 of SEQ ID NO: 3.

5. The isolated polypeptide of claim 1, which comprises an amino acid sequence that is up to 99% identical to SEQ ID NO: 4, comprising at least the sequence of amino acids 518 to 538 of SEQ ID NO: 4.

6. The isolated polypeptide of claim 2, which comprises the sequence of amino acids 99 to 492 of SEQ ID NO: 1.

7. The isolated polypeptide of claim 3, which comprises the sequence of amino acids 208 to 836 of SEQ ID NO: 2.

8. The isolated polypeptide of claim 3, which comprises the sequence of amino acids 1 to 836 of SEQ ID NO: 2.

9. The isolated polypeptide of claim 3, which comprises the sequence of amino acids 208 to 941 of SEQ ID NO: 2.

10. An isolated oligonucleotide, comprising an oligonucleotide sequence selected from the group consisting of:
a) an oligonucleotide sequence that is less than 50 nucleotides in length and that hybridizes under physiological conditions to an ATG14L nucleic acid coding sequence as set forth in SEQ ID NO: 5 or a complementary sequence thereof;
b) an oligonucleotide sequence that is less than 50 nucleotides in length and that hybridizes under physiological conditions to an ATG14L nucleic acid coding sequence as set forth in SEQ ID NO: 6 or a complementary sequence thereof;
c) an oligonucleotide sequence that is less than 50 nucleotides in length and that hybridizes under physiological conditions to a Rubicon nucleic acid coding sequence as set forth in SEQ ID NO: 7 or a complementary sequence thereof; and
d) an oligonucleotide sequence that is less than 50 nucleotides in length and that hybridizes under physiological conditions to a Rubicon nucleic acid coding sequence as set forth in SEQ ID NO: 8 or a complementary sequence thereof.

11. The isolated oligonucleotide of claim 10, which comprises a nucleotide sequence less than 50 nucleotides in length and that hybridizes under physiological conditions to an ATG14L nucleic acid coding sequence as set forth in SEQ ID NO: 5 or a complementary sequence thereof.

12. The isolated oligonucleotide of claim 10, which comprises a nucleotide sequence less than 50 nucleotides in length and that hybridizes under physiological conditions to an ATG14L nucleic acid coding sequence as set forth in SEQ ID NO: 6 or a complementary sequence thereof.

13. The isolated oligonucleotide of claim 10, which comprises a nucleotide sequence less than 50 nucleotides in length and that hybridizes under physiological conditions to a Rubicon nucleic acid coding sequence as set forth in SEQ ID NO: 7 or a complementary sequence thereof.

14. The isolated oligonucleotide of claim 10, which comprises a nucleotide sequence less than 50 nucleotides in length and that hybridizes under physiological conditions to a Rubicon nucleic acid coding sequence as set forth in SEQ ID NO: 8 or a complementary sequence thereof.

15. The isolated oligonucleotide of claim 10, which comprises the nucleotide sequence as set forth in SEQ ID NO: 11.

16. The isolated oligonucleotide of claim 10, which comprises the nucleotide sequence as set forth in SEQ ID NO: 12.
